SEQUENCE LISTING (22 sequences)

SEQ ID NO: 1 - protein, 314 aa, Unknown
  Cucurbitaceae
MEKIQHSTIATNSINIHLASIGSGPPVLLLHGFPELWYSWRHQLLFLASKGFRAIAPDLRGFGDSDVPSSPSSYTPHHIIGDLIGVLDHLGIDQVFLVGHDWGAMMAWYFCLFRPDRVKALVNLSVYYTPRNPVISPLAGLRLVLGDDFYICRFQEPGVAEADFGSVDAATMMKKFLTMRDPSPPIIPNGFKTLATPETLPPWLTEEDIDYFASKFGKTGFTGGFNYYRAIDLSWELTGPWNGVKIKVPTKFIVGDLDLVYHFPGAKEYIHGDGFKNDVPFLEEVVVIEGAAHFINQEKADEVSSLIYDFIKKF

SEQ ID NO: 2 - protein, 314 aa, Unknown; identical to SEQ ID NO: 1
  Cucurbitaceae

SEQ ID NO: 3 - protein, 474 aa, Unknown
  Cucurbitaceae
MWTILLGLATLAIAYYIHWVNKWKDSKFNGVLPPGTMGLPLIGETIQLSRPSDSLDVHPFIQSKVKRYGPIFKTCLAGRPVVVSTDAEFNHYIMLQEGRAVEMWYLDTLSKFFGLDTEWLKALGLIHKYIRSITLNHFGAESLRERFLPRIEESARETLHYWSTQPSVEVKESAAAMVFRTSIVKMFSEDSSKLLTGGLTKKFTGLLGGFLTLPLNLPGTTYNKCIKDMKEIQKKLKDILEERLAKGTGNDEDFLGQAIKDKESQQFISEEFIIQLLFSISFASFESISTSLTLILKFLADHPQVVKELEAEHEAIQKARADPDGPITWEEYKSMTFTLNVISETLRLGSVTPALLRKTTKEIQIKGYTIPEGWTVMLVTASRHRDPEVYKDPDTFNPWRWKELDSITVQKNFMPFGGGLRHCAGAEYSKVYLCTFLHILFTKYRWTKLKGGKIARAHILRFEDGLHVKFTPKE

SEQ ID NO: 4 - protein, 474 aa, Unknown
  Cucurbitaceae
MWTIFLGFVTLTIVYYIYWTNKWKDSKFNRVLPPGTMGLPLIGETIQLSRPSYSLDLHPFIQTKVKRYGPIFKTCLAGRPVVVSTDAEFNHYIMLQEGKAVEMWYLDAFSKFFGLDTEWLKALGLIHKYIRSITLNHYGVESLRERFLPRIEESVRETLHYWATQPSVEVKDSAAAMVLKTSVIKLFSEDSNKLLSGGLLKKFTKLLGGFLTLPLNLPGTTYHRCIKDMKEIQKKLKEILEERLARGNGSDEDFLGQVIKDKESQKFISEEFIIQLLFSMSFATFESISASLTLILILLADHPEVAKELEVEHEAIRKARVDPDGLITWEEYKSMTFTHNVINETLRLGSVTPSLLRKTTKEIQIKGYTIPEGWTIMLVIASRHRNPKVYKDPDTFNPWRWKELDSITIQKNFMPFGGGLRHCAGAEYTKVYLCTFLHILFTKYRWTKLKGGKIARALILKFVDGLHVNFTPKE

SEQ ID NO: 5 - protein, 442 aa, Unknown
  Cucurbitaceae
MICIAMDAQQATKTTILMFPWLGYGHLSAYLELAKALCSSSRNNFHIYFCSTSVNLDSIKPKLVPSSSIQFVELHLPSSPELPPHLHTANALPPHLMPTLHQAFANSAPHFEAILLKLSPHLLIYDCFQSWAPRLASSLNIPAINFNTSGVSLISHGFHAINYPDSKLPFSNFVLHNHWKAKYASPPSEHTQNVKEAFFYCLNASCDVILTNSFREVEGEYMDYLSLLSKKKVIPVGPLVYEPNEEEEDENYWRIKNWLDKKKALSTVLVSFGSESYASEEEKEEVGNGLEESEANFIWVERDHEQQRRGFVERAGERALVVKGWAPQAKILKHGSIGGFVSHCGWNSVLESIVFGVPIIGVPVFGDQPFNAGVVEEAGVGVEAKRDPDGKIQTQEVAKLIKQVVVEKTREDLRMKVREMSDVLRRKRDKRIDEMLAQISLV

SEQ ID NO: 6 - protein, 461 aa, Unknown
  Cucurbitaceae
MDVHKAKDKPTTILMFPWIGYGHLSAYLELAKAVSRCRNNFQIYFCSTPVNLDSIRPKLVASSSSSSSSSIQFVELHLPSSPEFPPHLHTTNALPPHLTPTLHQAFAAAAPRFESILQTLSPHLLIYDVLQPWAPRIASSLNIPAINFNTTATSIISHALHNIHYPNSKFPLSDFVLHNYWKAKYTNVDGTTTERGRRVREAFLYCLTASHDVILINSFKEMEGKYMDYLSVMLKKRVIAVGPLVYEPSEDEDEEDEDYSRIKNWLDKKEASSTVLVAFGSEFFPSKEEMEEIAIGLEKSGANFIWVVRFPKGEERKRIEELLPEGFVERAGERAMVEKDWAPQGKILKHRSIGGFVSHCGWNSVMESIMLGVPVIGVPMHVDQPYNAGLLEEAGVGVEAKRDPDGKIQRQEIANLIRQVVVDKSREDLRMKVREMSEILRRKGDQKIDEMLTQISLLLKI

SEQ ID NO: 7 - protein, 1100 aa, Unknown
  Cucurbitaceae
MAKPRVLLFPFPALGHVKPFLSLAELLSDAGLDVVFLNSEYNHRRMTNVIEFLSSRFPSIRFETIPDGLPPDQPRSLVDSPLYFTMRDGTKPRFRQLIHSFNDGCSANPWRITFIINDVMLCSPIEVAEEFGIPVISFCPHSARYFYTHFLVPKLVEEGQIPYTDKNPVGKIEGIPLFEGHLRRNHLPGSWSQQSSHISFSHSLINQTVAAARSSALILNTFHDLEAPFLTHLSSIFNKIYTIGPLHALFKSKLRNSSSPPPTLIGFWKDDESYISWLDSQPPGSVISISFGSSMKMEARELTEFWHGHIKPFLCLAHLLCNAGFRVTFLNTDHHHRNIHDLTRLAAQLPSLHFHSISDGLPPDQPQNLLDGKLFKSMPQVTKFLFRDLLLSYNNGTSPITCVITDVILRFPMDVAQQLGIPVFCFSTFSARFLFLYFSIPKLLEDGQIPYPEGNSNQQLYGIPGGEGLLRCKDLPGFWSVENVAKSNPMNFVNQTLATSKSCGLILNTFDELEAPFVTSLSKIYKKVYTIGPIHSLLKNSAQPQCELWKEDHSCLAWLDSQPPRSVVFVSFGSLVKVTSSQLKEFWNGLVNSGKAFVLVLRSDTRVRSIMEYQDEKMESTIAELAKRAKDRPSLSLLSMALSPSDHVLLFPFPAKGHIKPFLCLAHLLCNAGLRVTFLNTHHNHRHIHDLTRLAAQLPSLHFESISDGLPPDQPRNVLDGKLYESMHQVTKSLFRQLLISYNNGTSSTRPPITCLITDVIFRFPMDLAEELGIPVFCFSTFSARFLFLFFWIPKLLEDGQIPYPVGNSNQELHGVPGGEGILRCKDLPGFWSVEAVAKRDPMKFVNQTLATSKSCGIILNTFNELEAPFVTCLSEVYKKVYTIVPIHSLVKTTAQSQCEFWNEDHSCLAWLDSQPPRSVVFISFGSLVKLTSSQLMEFWNGLVNSGKAFVLVLRSDGLIEEVGEKDKKQKELVIREIMDTKAEGRWVIVNWAPQEEVLSHKAIGGFLTHSGWNSTLESLTAGVPMISWPHIGDQPSNATWLTKDSYDRSTIETMVRSIMEHQDEKMENAIVEWAKGAKDNVSKHGTSYQNLQCLIEDIK

SEQ ID NO: 8 - protein, 542 aa, Unknown
  Cucurbitaceae
MVDQCGLGWILASVLGAAALYFLFGKKNCGVSNERRRESLKNIATTNGECKSSNSDGDIIIVGAGVAGSALAYTLAKDGRRVHVIERDLTEPDRIVGELLQPGGYLKLTELGLEDCVNDIDAQRVYGYALFKDGKDTRLSYPLEKFHSDVSGRSFHNGRFIQRMREKAGSLPNVRLEQGTVTSLLEENGTIKEDFKYLNLFQVDVPSCFVGLVLENCDLPYANHGHVILADPSPILFYPISSTEIRCLVDVPGQKVPSISNGEMANYLKNVVAPQIPPQLYDSFIAAIDKGNIRTMPNRSMPADPYPTPGALLMGDAFNMRHPLTGGGMTVALSDIVVLRDLLKPLRDLNDAPTLCKYLEAFYTLRKPVASTINTLAGALYKVFCASPDQARKEMRQACFDYLSLGGIFSNGPVSLLSGLNPRPLSLVLHFFAVAIYGVGRLLIPFPSPKRVWIGARLISGASAIIFPIIKAEGVRQMFFPKTVAAYYRAPPVVRKRGFCGKPCWFRSEDVNRLTNYSFLSVALCDVSYPEPGVKQIPKEYN

SEQ ID NO: 9 - protein, 582 aa, Unknown
  Cucurbitaceae
MWRLKVGAESVGEKEEKWLKSISNHLGRQVWEFCAHQPTASPNHLQQIDNARNHFRNNRFHRKQSSDLFLAIQNEKEIANVTKGGGIKVKEEEDVRKETVKNTVERALNADGGEGGAMTKARSWILDRGGATAITSWGKLWLSVLGVYEWSGNNPLPPEFWLLPYCLPFHPGRMWCHCRMVYLPMSYLYGKRFVGPITPIVLSLRKELYTIPYHEIDWNRSRNTCAKEDLYYPHPKMQDILWGSIYHLYEPLFTRWPGKRLREKALQMAMKHIHYEDENSRYICLGPVNKVLNMLCCWVEDPYSDAFKFHLQRVPDYLWVAEDGMRMQGYNGSQLWDTAFSVQAIISTKLIDSFGTTLKKAHDFVKDSQIQQDCPGDPNVWFRHIHKGAWPFSTRDHGWLISDCTAEGLKNENGGFASYELTRSYPWLELINPAETFGDIVIDYPYVECTSATMEALTLYGCWGVCFTYAGWFGIKGLVAAGRTYNSCVAIRKACDFLLSKELPGGGWGESYLSCQNKAERDPAPLHRAARLLINSQLENGDFPQEEIMGVFNKNCMITYAAYRNIFPIWALGEYFHRVLTD

SEQ ID NO: 10 - protein, 537 aa, Unknown
  Cucurbitaceae
MYVAVAASPPHKFYKTAFSFSIFTPRSDFSFVVAAAGFSPEETMVDHCAFGWILSAFLAFAIAFSFFLASRRNRRVGGADSTPRTDCVRSSASTNGECRSVDGDADVIIVGAGVAGSALAHTLGKDGRRVHVIERDLTEPDRIVGELLQPGGYLKLIELGLQDCVEEIDAQKVYGYALFKDGKNTRLSYPLENFHSDVSGRSFHNGRFIQRMRKKAASLPNVRLEQGTVTSLLEKKGTIKGVDVPSCFVGLVLENCQLPFANHGHVVLGDPSPILFYPISSTEIRCLVDVPGQKVPSISNGEMEKYLKTVVAPQVPPEIHDAFIAAIEKGNIRTMPNRSMPAAPQPTPGALLMGDAFNMRHPLTGGGMTVALSDIVVLRNLIKPLKDLNDAPTLCKYLESFYTLRMPVASTINTLAGALYKVFCASPDQARKEMRQACFDYLSLGGIFSNGPVSLLSGLNPRPLSLVLHFFAVAIYGVGRLLLPFPSLKGIWIGARLIYSASGIIFPIIKAEGVRQMFFPATVPAYYRTPPVFKPIA

SEQ ID NO: 11 - protein, 757 aa, Unknown
  Cucurbitaceae
MKSETQRSSESGRRSKRNMWRLRIAVGGNDPYIYSTNNFIGRQTWEFDPEAGTPKEREEAEQARLHFYQNRFKVKPSADILWRMQFLREKKFKQEIGAVKIKEGEEITEEKGRRALRRAVHFYSALQASDGHWPAENAGPLFFLPPLVMCVYITGHLDHVFSKEHKKEILRYIYCHQNEDGGWGLHIEGHSTMFCTALNYICMRILGEPSHGGINDACARARNWILDHGGVTYIPSWGKTWLSIFGVYDWAGSNPIPPEFWILPSFLPMHPAKMWCYCRMVYMPMSYLYGKRFVCQITPLIQELRQELHAQPFDKINWKKTRHLCTKEDLYYPHPLIQDLLWDSLYICTEPLLTRWPLNKLVREKALQVTMKHIHYEDENSRYITIGCVEKVLCMLACWVEDPNGDSFKKHLARIPDYIWVAEDGMKMQSFGSQEWDTGFAIQALLAADMADEIGPTLARGHDFIKNSQVKDNPSGDFNTMHRHISKGSWTFSDQDHGWQVSDCTAEALKCCLLFSMMKPEIFGEKMEDQRLWDTVNVLLSLQSKNGGLAAWEPAPTGQEWLEMMNPTEFFADIVIEHEYVECTASAIQALVLFKKLHPGHRTKEIDNFIQNAVRYLQDIQMPDGSWYGNWGVCFTYGGWFALGALVAAGKTFNNSASIRKARYVPLESNTSNLVQTAWAMLGLIHSGQAERDPGPLHRAAKLLINSQTEDGDFPQQEITGVFMKNCMLHYAAYRNIYPIWALAEYSKRVKFLSTSC

SEQ ID NO: 12 - protein, 684 aa, Unknown
  Cucurbitaceae
MWQLKIGADTLPADPSNAGGWLSTLNNHVGRQVWHFHPELGSPEDLQQIQHARQRFSDHRFEKKHSADLLMRMQFAKENSSFVNLPQIKVKDKEDVVEEAVTRTLRRAINFYSTIQAHDGHWPGDYGGPMFLMPGLNRDGGWGLHIEGPSTMFGSVLNYVTLRLLGEEAEDGQGGVDKARKWILDHGGATAITSWGKMWLSVLGVYEWAGNNPLPPELWLLPYFLPCHPGRMWCHCRMVYLPMCYLYGKRFVGPITPIIRSLRKELYLVPYHEVDWNEARNQCAKEDLYYPHPLVQDVLWASLHHVYEPLFMRWPAKRLREKALRSVMQHIHYEDENTRYICIGPVNKGYNGSQLWDTAFAVQAIMSTKLAEEYGTTLRKAHKYIKDSQVLEDCPGDLQNWYRHISKGAWPFSTADHGWPISDCTAEGFVINLSGVTRHVVNLFGVTSLQNTDGGFATYELTRSYQWLELMNPAETFGDIVIDYPYVECTSAAIQALAAFKKNYPGHRSNEIGNCIAKAADFIESIQATDGSWYGSWGVCFTYGGWFGIRGLIAAGRRYENCSSLRKACDFLLSKELAAGGWGESYLSCQDKVYTNIKDDRPHIVNTGWAMLSLIDAGQAERDPTPLHRAARILINSQMDDGDFPQEEIMGVFNKNCMISYSAYRNIFPIWALGEYRCRVLQSP

SEQ ID NO: 13 - protein, 316 aa, Unknown
  Cucurbitaceae
MEAIEHSTINTNGIKMHIASIGNGPVVLLLHGFPELWYSWRHQLLYLSSVGYRAIAPDLRGYGDTDSPQSHTSYTALHIVGDLVGALDELGIEKVFLVGHDWGAIIAWYFCLFRPDRIKALVNLSVHFLPRNPAFSFLQGFRAALGDDFYICRFQVPGEAEEQFASIDTTDLFKRFLCGRSPNPPRLPKEDGIRSIPPPQNLPSWLTEEDINYFATKFKHTGFTGGLNYYRALDLTWELTASWTKTQVRVPVKFVVGDLDLTYHFPGAKEYIHNGGFKRDVPFLEEVVVMEDTAHFINQERPHEISTQIHDFIKKF

SEQ ID NO: 14 - protein, 316 aa, Unknown
  Cucurbitaceae
MDAIEHSTINTNGIKMHIASIGNGPVVLLLHGFPELWYSWRHQLLYLSSVGYRAIAPDLRGYGDTDSPESHTSYTALHVVGDLVGALDELGIEKVFLVGHDWGAIIAWYFCLFRPERIKALVNMSVQFFPRNPAISFIERFRAVYGDDFYMCRFQVPGEAEADFACIDTAQLFKTTLSNKSTHAPCLPKEYGFRAIPPPENLPSWLTEEDINYYAAKFKETGFTGALNYYRAFDLTWELTGPWTGARIQVPVKFIVGDSDLTYHFKGAKEYIHEGGFKRDVPLLEEVVIVENAGHFVHEEKPHEINTHIHDFIKKF

SEQ ID NO: 15 - protein, 759 aa, Unknown
  Cucurbitaceae
MWRLKVGAESVGENDEKWLKSISNHLGRQVWEFCPDAGTQQQLLQVHKARKAFHDDRFHRKQSSDLFITIQYGKEVENGGKTAGVKLKEGEEVRKEAVESSLERALSFYSSIQTSDGNWASDLGGPMFLLPGLVIALYVTGVLNSVLSKHHRQEMCRYVYNHQNEDGGWGLHIEGPSTMFGSALNYVALRLLGEDANAGAMPKARAWILDHGGATGITSWGKLWLSVLGVYEWSGNNPLPPEFWLFPYFLPFHPGRMWCHCRMVYLPMSYLYGKRFVGPITPIVLSLRKELYAVPYHEIDWNKSRNTCAKEDLYYPHPKMQDILWGSLHHVYEPLFTRWPAKRLREKALQTAMQHIHYEDENTRYICLGPVNKVLNLLCCWVEDPYSDAFKLHLQRVHDYLWVAEDGMKMQGYNGSQLWDTAFSIQAIVSTKLVDNYGPTLRKAHDFVKSSQIQQDCPGDPNVWYRHIHKGAWPFSTRDHGWLISDCTAEGLKAALMLSKLPSETVGESLERNRLCDAVNVLLSLQNDNGGFASYELTRSYPWLELINPAETFGDIVIDYPYVECTSATMEALTLFKKLHPGHRTKEIDTAIVRAANFLENMQRTDGSWYGCWGVCFTYAGWFGIKGLVAAGRTYNNCLAIRKACDFLLSKELPGGGWGESYLSCQNKVYTNLEGNRPHLVNTAWVLMALIEAGQAERDPTPLHRAARLLINSQLENGDFPQQEIMGVFNKNCMITYAAYRNIFPIWALGEYCHRVLTE

SEQ ID NO: 16 - protein, 473 aa, Unknown
  Cucurbitaceae
MWTVVLGLATLFVAYYIHWINKWRDSKFNGVLPPGTMGLPLIGETIQLSRPSDSLDVHPFIQKKVERYGPIFKTCLAGRPVVVSADAEFNNYIMLQEGRAVEMWYLDTLSKFFGLDTEWLKALGLIHKYIRSITLNHFGAEALRERFLPFIEASSMEALHSWSTQPSVEVKNASALMVFRTSVNKMFGEDAKKLSGNIPGKFTKLLGGFLSLPLNFPGTTYHKCLKDMKEIQKKLREVVDDRLANVGPDVEDFLGQALKDKESEKFISEEFIIQLLFSISFASFESISTTLTLILKLLDEHPEVVKELEAEHEAIRKARADPDGPITWEEYKSMTFTLQVINETLRLGSVTPALLRKTVKDLQVKGYIIPEGWTIMLVTASRHRDPKVYKDPHIFNPWRWKDLDSITIQKNFMPFGGGLRHCAGAEYSKVYLCTFLHILCTKYRWTKLGGGTIARAHILSFEDGLHVKFTPKE

SEQ ID NO: 17 - protein, 316 aa, Unknown
  Cucurbitaceae
MDQIEHITINTNGIKMHIASVGTGPVVLLLHGFPELWYSWRHQLLYLSSVGYRAIAPDLRGYGDTDSPASPTSYTALHIVGDLVGALDELGIEKVFLVGHDWGAIIAWYFCLFRPDRIKALVNLSVQFIPRNPAIPFIEGFRTAFGDDFYMCRFQVPGEAEEDFASIDTAQLFKTSLCNRSSAPPCLPKEIGFRAIPPPENLPSWLTEEDINYYAAKFKQTGFTGALNYYRAFDLTWELTAPWTGAQIQVPVKFIVGDSDLTYHFPGAKEYIHNGGFKKDVPLLEEVVVVKDACHFINQERPQEINAHIHDFINKF

SEQ ID NO: 18 - protein, 528 aa, Unknown
  Cucurbitaceae
MVDQCALGWILASALGLVIALCFFVAPRRNHRGVDSKERDECVQSAATTKGECRFNDRDVDVIVVGAGVAGSALAHTLGKDGRRVHVIERDLTEPDRIVGELLQPGGYLKLIELGLQDCVEEIDAQRVYGYALFKDGKNTRLSYPLENFHSDVSGRSFHNGRFIQRMREKAASLPNVRLEQGTVTSLLEEKGTIKGVQYKSKNGEEKTAYAPLTIVCDGCFSNLRRSLCNPMVDVPSYFVGLVLENCELPFANHGHVILGDPSPILFYQISRTEIRCLVDVPGQKVPSIANGEMEKYLKTVVAPQVPPQIYDSFIAAIDKGNIRTMPNRSMPAAPHPTPGALLMGDAFNMRHPLTGGGMTVALSDIVVLRNLLKPLKDLSDASTLCKYLESFYTLRKPVASTINTLAGALYKVFCASPDQARKEMRQACFDYLSLGGIFSNGPVSLLSGLNPRPLSLVLHFFAVAIYGVGRLLLPFPSVKGIWIGARLIYSASGIIFPIIRAEGVRQMFFPATVPAYYRSPPVFKPIV

SEQ ID NO: 19 - protein, 474 aa, Unknown
  Cucurbitaceae
MVQPRVLLFPFPALGHVKPFLSLAELLSDAGIDVVFLSTEYNHRRISNTEALASRFPTLHFETIPDGLPPNESRALADGPLYFSMREGTKPRFRQLIQSLNDGRWPITCIITDIMLSSPIEVAEEFGIPVIAFCPCSARYLSIHFFIPKLVEEGQIPYADDDPIGEIQGVPLFEGLLRRNHLPGSWSDKSADISFSHGLINQTLAAGRASALILNTFDELEAPFLTHLSSIFNKIYTIGPLHALSKSRLGDSSSSASALSGFWKEDRACMSWLDCQPPRSVVFVSFGSTMKMKADELREFWYGLVSSGKPFLCVLRSDVVSGGEAAELIEQMAEEEGAGGKLGMVVEWAAQEKVLSHPAVGGFLTHCGWNSTVESIAAGVPMMCWPILGDQPSNATWIDRVWKIGVERNNREWDRLTVEKMVRALMEGQKRVEIQRSMEKLSKLANEKVVRGGLSFDNLEVLVEDIKKLKPYKF

SEQ ID NO: 20 - protein, 452 aa, Unknown
  Cucurbitaceae
MDAAQQGDTTTILMLPWLGYGHLSAFLELAKSLSRRNFHIYFCSTSVNLDAIKPKLPSSFSDSIQFVELHLPSSPEFPPHLHTTNGLPPTLMPALHQAFSMAAQHFESILQTLAPHLLIYDSLQPWAPRVASSLKIPAINFNTTGVFVISQGLHPIHYPHSKFPFSEFVLHNHWKAMYSTADGASTERTRKRGEAFLYCLHASCSVILINSFRELEGKYMDYLSVLLNKKVVPVGPLVYEPNQDGEDEGYSSIKNWLDKKEPSSTVFVSFGSEYFPSKEEMEEIAHGLEASEVNFIWVVRFPQGDNTSGIEDALPKGFLERAGERGMVVKGWAPQAKILKHWSTGGFVSHCGWNSVMESMMFGVPIIGVPMHVDQPFNAGLVEEAGVGVEAKRDPDGKIQRDEVAKLIKEVVVEKTREDVRKKAREMSEILRSKGEEKFDEMVAEISLLLKI

SEQ ID NO: 21 - protein, 473 aa, Unknown
  Cucurbitaceae
MWTVVLGLATLFVAYYIHWINKWRDSKFNGVLPPGTMGLPLIGETIQLSRPSDSLDVHPFIQKKVERYGPIFKTCLAGRPVVVSADAEFNNYIMLQEGRAVEMWYLDTLSKFFGLDTEWLKALGLIHKYIRSITLNHFGAEALRERFLPFIEASSMEALHSWSTQPSVEVKNASALMVFRTSVNKMFGEDAKKLSGNIPGKFTKLLGGFLSLPLNFPGTTYHKCLKDMKEIQKKLREVVDDRLANVGPDVEDFLGQALKDKESEKFISEEFIIQLLFSISFASFESISTTLTLILKLLDEHPEVVKELEAEHEAIRKARADPDGPITWEEYKSMTFTLQVINETLRLGSVTPALLRKTVKDLQVKGYIIPEGWTIMLVTASRHRDPKVYKDPHIFNPWRWKDLDSITIQKNFMPFGGGLRHCAGAEYSKVYLCTFLHILCTKYRWTKLGGGRIARAHILSFEDGLHVKFTPKE

SEQ ID NO: 22 - protein, 163 aa, Chlamydomonas reinhardtii
MNTKYNKEFLLYLAGFVDGDGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

(19) United States
(12) Patent Application Publication
(10) Pub. No.: US 2023/0263121 A1
(43) Pub. Date: Aug. 24, 2023

(54) MODULATION OF ENDOGENOUS MOGROSIDE PATHWAY GENES IN WATERMELON AND OTHER CUCURBITS

(71) Applicant: Elo Life Systems, Durham, NC (US)

(72) Inventors: Tengfang Huang, Cary, NC (US); Jing Zhou, Cary, NC (US); Hye-ran Lee, Chapel Hill, NC (US); Jack Q. Wilkinson, Cary, NC (US); Fayaz Khazi, Chapel Hill, NC (US)

(21) Appl. No.: 17/907,742

(22) PCT Filed: Mar. 30, 2021
(86) PCT No.: PCT/US2021/024860

§ 371 Date: Sep. 29, 2022

Related U.S. Application Data
(60) Provisional application No. 63/002,796, filed on Mar. 31, 2020.

(51) Int. Cl. A01H 5/08; A01H 6/34

(52) U.S. Cl. CPC A01H 5/08; A01H 6/34

(57) ABSTRACT

Provided herein are methods for increasing the level of mogrosides in plants, such as in Cucurbitaceae family plants (e.g., in watermelon), by genetically-modifying such plants to modulate the expression of endogenous mogroside pathway genes. Also disclosed herein are plants with increased levels of mogrosides, extract (e.g., sweetener) from such plants, parts (e.g., juice, seed, pulp, etc.) from such plants, and methods of producing such plants.

### FIELD OF THE INVENTION

The invention relates to methods for increasing the level of mogrosides in plants by genetically-modifying such plants to modulate the expression of endogenous mogroside pathway genes. The invention further relates to plants, plant parts, and plant products containing increased levels of mogrosides.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Mar. 30, 2021, is named PBIO-0101.8W0 Seq List 3-31 and is 95,189 bytes in size.

### INCORPORATION OF SUBSTITUTE SEQUENCE LISTING

The substitute sequence listing that is contained in the file named “ELSS005US_ST25.txt,” which is 94 kilobytes (size as measured in Microsoft Windows® operating system) and was created on Dec. 21, 2022, is filed electronically herewith and is incorporated by herein by reference.

### BACKGROUND

Sweetness is one of the fundamental human hedonic pleasures, even more reinforcing and attractive than drugs such as heroin and cocaine (Madsen and Ahmed Addict Biol 20:433-444 (2015)). However, in satisfying this desire, sugar consumption has risen exponentially from nearly 250 years ago and meta-analyses implicate sugar consumption in the development of obesity, diabetes, metabolic syndrome, and cardiovascular diseases (Bray and Popkin, Diabetes Care 37:950-956 (2014)). In light of potential advantages associated with alternative natural sweeteners, there is great interest in developing alternative natural nonsugar sweeteners to satisfy the human desire for sweet flavors.

Natural compounds with strong sweetening capacity belong to numerous chemical families, including proteins, flavonoids, and terpenoids (Kim and Kinghorn, Arch Pharm Res 25:725-746 (2002)). The mogroside family of triterpenoids, derived from the ripe fruit of the Cucurbitaceae family plant Siraitia grosvenorii (iuo-han-guo or monk fruit, discovered and classified initially in the 1930s (Swingle, J Arnold Arbor 22:197-203 (1941)), is used as a natural sweetener in China, having a sweetening strength of 250 times that of sucrose (Kasai et al., Agric Biol Chem 53:3347-3349 (1989)). Moreover, additional health benefits of mogrosides have been revealed in recent studies (Li et al., Chin J Nat Med 12:89-102 (2014)). Overall, mogrosides derived from monk fruit have been regarded as acceptable natural, zero-calorie sweeteners for the food and beverage applications and currently have GRAS status (Ibrahim, EC Nutrition 1:57-56 (2015); see the website at fda.gov/media/109982/download).

However, adoption of mogrosides for every day consumption and their use by the food industry have been sluggish for various reasons. First, monk fruit is indigenous to parts of Asia, with majority of the production taking place in China. Furthermore, monk fruits are grown using traditional practices, resulting in very high production costs. Moreover, poor productivity of monk fruits, variable levels of mogrosides in the fruits, and poor understanding of monk fruit biology also hinder the wide scale use of mogrosides. In view of the difficulty with in planter production of mogrosides by monk fruits due to the difficulty to grow these plants outside of their native habitat, there is an unmet need to produce mogrosides in widely adapted plant systems.

### SUMMARY OF THE INVENTION

Provided herein are methods for increasing production of mogrosides in plants, such as in plants of the Cucurbitaceae family, by genetically-modifying the genome of plants to modulate the expression of endogenous pathway for mogroside biosynthesis. Also disclosed herein are plants (e.g., a plant belonging to the Cucurbitaceae family, such as a watermelon plant) with increased production of mogrosides, extract (e.g., sweetener) from such plants, parts (e.g., juice, seed, pulp, etc.) from such plants, plant concentrate (e.g., whole plant concentrate or plant part concentrate) from such plants, plant powder (e.g., formulated powder, such as formulated plant part powder) from such plants, plant biomass (e.g., dried biomass, such as crushed and/or powdered biomass) from such plants, and methods of producing such plants or progeny of such plants.

Accordingly, in one aspect, the invention provides a method for increasing the level of one or more mogrosides in a cell by genetically-modifying the genome of the cell to modulate the expression of one or more endogenous mogroside synthesis pathway genes.

In some embodiments of the method, the one or more endogenous mogroside synthesis pathway genes are selected from an endogenous gene encoding cucurbitadienol synthase (CDS), an endogenous gene encoding cytochrome P450 enzyme 87D18 (CYP87D18), an endogenous gene encoding epoxide hydrolase 3 (EPH3), an endogenous gene encoding squalene epoxidase 1 (SQE1), an endogenous gene encoding UDP glycosyltransferase 720 (UGT720), an endogenous gene encoding UDP glycosyltransferase 94 (UGT94), or one or more homologs thereof.

In some embodiments of the method, the one or more endogenous mogroside synthesis pathway genes are selected from endogenous genes encoding polypeptide sequences having at least 80% sequence identity to the amino acid sequence set forth in any one of SEQ ID NOs: 1-14 and 21.

In some embodiments, the method comprises modulating the expression of one or more endogenous mogroside synthesis pathway genes in genome of a Cucurbitaceae family plant. In some such embodiments of the method, the Cucurbitaceae family plant is a watermelon plant or a cucumber plant. In some such embodiments of the method, the watermelon plant is a Charleston Gray variety watermelon plant.

In some embodiments of the method, modulating the expression of the one or more endogenous mogroside synthesis pathway genes comprises modification of promoter sequences of the genes. In some such embodiments of the method, the modification of promoter sequences comprises: (i) insertion of one or more nucleotide sequences; and/or (ii) deletion of one or more nucleotide sequences. In certain embodiments of the method, the modification of promoter sequences comprises enhancing, improving, or increasing the activity of the promoter sequences by: (i) insertion of one or more nucleotide sequences; and/or (ii) deletion of one or more nucleotide sequences. In some such embodiments of the method, the modification of promoter sequences comprises replacement of promoter sequences with cisgenic or transgenic nucleic acid sequences, such as cisgenic or transgenic promoters.

In some such embodiments of the method, the modification of promoter sequences comprises correction or editing of promoter sequences. In certain embodiments of the method, the modification of promoter sequences comprises: (i) detection of one or more polymorphism or mutation that limits the activity of promoter sequences; and (ii) correction of the promoter sequences by deletion or correction of the polymorphism or mutation.

In some embodiments of the method, the modification of promoter sequences comprises insertion, deletion, and/or modification of one or more upstream nucleotide sequences. In certain embodiments of the method, the modification of promoter sequences comprises enhancing or improving or increasing the activity of the promoter sequences by insertion, deletion, and/or modification of one or more upstream nucleotide sequences.

In some such embodiments of the method, the modification of promoter sequences comprises addition, insertion, and/or engineering of cis-acting factors. In certain embodiments of the method, the cis-acting factors interact with and modify the promoter sequences. In some embodiments of the method, modulation of expression of one or more transcription factor genes modulates expression of the one or more endogenous mogroside synthesis pathway genes. In some such embodiments of the method, the modulation of expression of the one or more transcription factor genes activates promoter sequences of the one or more endogenous mogroside synthesis pathway genes. In some such embodiments of the method, the modulation of expression of the one or more transcription factor genes increases the expression of one or more endogenous mogroside synthesis pathway genes.

In some embodiments of the method, modulation of the expression of the one or more endogenous mogroside synthesis pathway genes comprises modifying negative regulatory sequences of the genes. In some such embodiments of the method, modulation of the expression of the one or more endogenous mogroside synthesis pathway genes comprises modifying negative regulatory sequences of the genes in a cis or trans location. In certain embodiments of the method, the negative regulatory sequences comprise upstream open reading frames (uORFs). In some embodiments of the method, modulation of the expression of the one or more endogenous mogroside synthesis pathway genes comprises insertion, modification, and/or engineering of transcription factor binding sites or enhancer elements. In certain embodiments of the method, modulation of the expression of the one or more endogenous mogroside synthesis pathway genes comprises insertion of novel transcription factor binding sites or enhancer elements. In certain embodiments of the method, modulation of the expression of the one or more endogenous mogroside synthesis pathway genes comprises modification and/or engineering of existing transcription factor binding sites or enhancer elements.

In some embodiments of the method, genetically-modifying the genome of the cell to modulate the expression of the one or more endogenous mogroside synthesis pathway genes comprises insertion of one or more functional promoters into the genome of the cell, wherein following said insertion, the one or more functional promoters are operably linked to the one or more endogenous mogroside synthesis pathway genes. In certain embodiments of the method, the one or more functional promoters are homologous promoters. In certain embodiments of the method, the one or more functional promoters are heterologous promoters.

In some such embodiments of the method, the promoter is a cassava vein mosaic virus (CSVMV) promoter. In certain embodiments of the method, the CSVMV promoter is operably linked to a gene encoding CDS, CYP87D18, EPH3, SQE, UGT720, or UGT94.

In some embodiments of the method, the one or more mogrosides is selected from mogrol, mogroside I-Al, mogroside I-El, mogroside IIA, mogroside IIB, mogroside IIE, 7-oxomogroside IIE, 11-oxomogroside Al, mogroside III, mogroside III Al, mogroside III A2, mogroside IIIx, 11-deoxymogroside III, mogroside IV, mogroside IV-A, 11-oxomogroside IV-A, siamenoside I, mogroside V, 7-oxomogroside V, 11-oxomogroside V, mogroside VI, and mogroside VII. In certain embodiments of the method, the one or more mogrosides is mogrol. In certain embodiments of the method, the one or more mogrosides is mogroside I-Al. In certain embodiments of the method, the one or more mogrosides is mogroside I-EL In certain embodiments of the method, the one or more mogrosides is mogroside IIA. In certain embodiments of the method, the one or more mogrosides is mogroside IIB. In certain embodiments of the method, the one or more mogrosides is mogroside IIE. In certain embodiments of the method, the one or more mogrosides is 7-oxomogroside IIE. In certain embodiments of the method, the one or more mogrosides is 11-oxomogroside Al. In certain embodiments of the method, the one or more mogrosides is mogroside III. In certain embodiments of the method, the one or more mogrosides is mogroside III Al. In certain embodiments of the method, the one or more mogrosides is mogroside III A2. In certain embodiments of the method, the one or more mogrosides is mogroside Mx. In certain embodiments of the method, the one or more mogrosides is 11-deoxymogroside III. In certain embodiments of the method, the one or more mogrosides is mogroside IV. In certain embodiments of the method, the one or more mogrosides is mogroside IV-A. In certain embodiments of the method, the one or more mogrosides is 11-oxomogroside IV-A. In certain embodiments of the method, the one or more mogrosides is siamenoside I. In certain embodiments of the method, the one or more mogrosides is mogroside V. In certain embodiments of the method, the one or more mogrosides is 7-oxomogroside V. In certain embodiments of the method, the one or more mogrosides is 11-oxomogroside V. In certain embodiments of the method, the one or more mogrosides is mogroside VI. In certain embodiments of the method, the one or more mogrosides is mogroside VII.

In some embodiments of the method, the cell produces an increase of at least 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more, of the one or more mogrosides when compared to a control cell; and/or the cell produces an increase about 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more, of the one or more mogrosides when compared to a control cell.

In some such embodiments of the method, the cell produces an increase of at least 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more of mogroside V, when compared to a control cell; and/or the cell produces an increase of about 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more, of mogroside V, when compared to a control cell.

In some embodiments of the method, genetically-modifying the genome of the cell to modulate the expression of the one or more endogenous mogroside synthesis pathway genes comprises increasing the expression of said one or more endogenous mogroside synthesis pathway genes.

In another aspect, the invention provides a method for producing a plant comprising increased levels of one or more mogrosides, by genetically-modifying the genome of a plant cell or plant part to modulate the expression of one or more endogenous mogroside synthesis pathway genes, and growing a plant from said plant cell or plant part, wherein the plant comprises increased levels of one or more mogrosides compared to a control plant.

In some embodiments of the method, the one or more endogenous mogroside synthesis pathway genes are selected from an endogenous gene encoding cucurbitadienol synthase (CDS), an endogenous gene encoding cytochrome P450 enzyme 87D18 (CYP87D18), an endogenous gene encoding epoxide hydrolase 3 (EPH3), an endogenous gene encoding squalene epoxidase 1 (SQE1), an endogenous gene encoding UDP glycosyltransferase 720 (UGT720), an endogenous gene encoding UDP glycosyltransferase 94 (UGT94), or one or more homologs thereof.

In some embodiments of the method, the one or more endogenous mogroside synthesis pathway genes are selected from endogenous genes encoding polypeptide sequences having at least 80% sequence identity to the amino acid sequence set forth in any one of SEQ ID NOs: 1-14 and 21.

In some embodiments of the method, genetically-modifying the genome of a plant cell or plant part to modulate the expression of the one or more endogenous mogroside synthesis pathway genes comprises modification of promoter sequences of the genes. In some such embodiments of the method, the modification of promoter sequences comprises: (i) insertion of one or more nucleotide sequences; and/or (ii) deletion of one or more nucleotide sequences.

In some embodiments of the method, modulation of the expression of the one or more endogenous mogroside synthesis pathway genes comprises modulation of expression of one or more transcription factor genes. In some such embodiments of the method, the modulation of expression of the one or more transcription factor genes activates promoter sequences of the one or more endogenous mogroside synthesis pathway genes. In some such embodiments of the method, the modulation of expression of the one or more transcription factor genes increases the expression of the one or more endogenous mogroside synthesis pathway genes.

In some embodiments of the method, modulation of the expression of the one or more mogroside synthesis pathway genes comprises modifying negative regulatory sequences of the genes. In some embodiments of the method, the negative regulatory sequences comprise upstream open reading frames (uORFs).

In some embodiments of the method, genetically-modifying the genome of a plant cell or plant part to modulate the expression of the one or more endogenous mogroside synthesis pathway genes comprises insertion of one or more functional promoters into the genome of the plant cell or plant part, wherein following said insertion, the one or more functional promoters are operably linked to said one or more mogroside synthesis pathway genes. In certain embodiments of the method, the one or more functional promoters are homologous promoters. In certain embodiments of the method, the one or more functional promoters are heterologous promoters.

In some such embodiments of the method, the promoter is a cassava vein mosaic virus (CSVMV) promoter. In certain embodiments of the method, the CSVMV promoter is operably linked to a gene encoding CDS, CYP87D18, EPH3, SQE, UGT720, or UGT94.

In some embodiments of the method, the one or more mogrosides is selected from mogrol, mogroside I-Al, mogroside I-El, mogroside IIA, mogroside IIB, mogroside IIE, 7-oxomogroside IIE, 11-oxomogroside Al, mogroside III, mogroside III Al, mogroside III A2, mogroside Mx, 11-deoxymogroside III, mogroside IV, mogroside IV-A, 11-oxomogroside IV-A, siamenoside I, mogroside V, 7-oxomogroside V, 11-oxomogroside V, mogroside VI, and mogroside VII. In certain embodiments of the method, the one or more mogrosides is mogrol. In certain embodiments of the method, the one or more mogrosides is mogroside I-Al. In certain embodiments of the method, the one or more mogrosides is mogroside I-El. In certain embodiments of the method, the one or more mogrosides is mogroside IIA. In certain embodiments of the method, the one or more mogrosides is mogroside IIB. In certain embodiments of the method, the one or more mogrosides is mogroside IIE. In certain embodiments of the method, the one or more mogrosides is 7-oxomogroside IIE. In certain embodiments of the method, the one or more mogrosides is 11-oxomogroside Al. In certain embodiments of the method, the one or more mogrosides is mogroside III. In certain embodiments of the method, the one or more mogrosides is mogroside III Al In certain embodiments of the method, the one or more mogrosides is mogroside III A2. In certain embodiments of the method, the one or more mogrosides is mogroside Mx. In certain embodiments of the method, the one or more mogrosides is 11-deoxymogroside III. In certain embodiments of the method, the one or more mogrosides is mogroside IV. In certain embodiments of the method, the one or more mogrosides is mogroside IV-A. In certain embodiments of the method, the one or more mogrosides is 11-oxomogroside IV-A. In certain embodiments of the method, the one or more mogrosides is siamenoside I. In certain embodiments of the method, the one or more mogrosides is mogroside V. In certain embodiments of the method, the one or more mogrosides is 7-oxomogroside V. In certain embodiments of the method, the one or more mogrosides is 11-oxomogroside V. In certain embodiments of the method, the one or more mogrosides is mogroside VI. In certain embodiments of the method, the one or more mogrosides is mogroside VII.

In some embodiments of the method, modulating the expression of the one or more mogroside synthesis pathway genes comprises increasing the expression of the one or more endogenous mogroside synthesis pathway genes.

In some embodiments of the method, the plant is a Cucurbitaceae family plant. In some such embodiments of the method, the plant is a watermelon plant or cucumber plant. In certain embodiments of the method, the plant is a Charleston Gray variety watermelon plant.

In another aspect, the invention provides a plant or plant part comprising increased levels of at least one mogroside compared to a control plant, wherein the genome of the plant comprises a genetic modification to modulate the expression of one or more endogenous mogroside synthesis pathway genes.

In some embodiments of the plant or plant part, the one or more endogenous mogroside synthesis pathway genes are selected from an endogenous gene encoding cucurbitadienol synthase (CDS), an endogenous gene encoding cytochrome P450 enzyme 87D18 (CYP87D18), an endogenous gene encoding epoxide hydrolase 3 (EPH3), an endogenous gene encoding squalene epoxidase 1 (SQE1), an endogenous gene encoding UDP glycosyltransferase 720 (UGT720), an endogenous gene encoding UDP glycosyltransferase 94 (UGT94), or one or more homologs thereof.

In some embodiments of the plant or plant part, the one or more mogroside synthesis pathway genes are selected from endogenous genes encoding polypeptide sequences having at least 80% sequence identity to the amino acid sequence set forth in any one of SEQ ID NOs: 1-14 and 21.

In some embodiments of the plant or plant part, the genetic modification to modulate the of expression of one or more endogenous mogroside synthesis pathway genes comprises a genetic modification of a promoter sequence of the one or more endogenous mogroside synthesis pathway genes. In some such embodiments of the plant or plant part, the genetic modification of a promoter sequence comprises: (i) insertion of one or more nucleotide sequences; and/or (ii) deletion of one or more nucleotide sequences.

In some embodiments of the plant or plant part, the modulation of expression of one or more endogenous mogroside synthesis pathway genes comprises modulation of expression of one or more transcription factor genes. In some such embodiments of the plant or plant part, the modulation of expression of the one or more transcription factor genes activates promoter sequences of the one or more endogenous mogroside synthesis pathway genes. In some embodiments of the plant or plant part, the modulation of expression of the one or more transcription factor genes increases the expression of the one or more endogenous mogroside synthesis pathway genes.

In some embodiments of the plant or plant part, the modulation of expression of the one or more endogenous mogroside synthesis pathway genes comprises modifying negative regulatory sequences of the genes. In some such embodiments of the plant or plant part, the negative regulatory sequences comprise upstream open reading frames (uORFs).

In some embodiments of the plant or plant part, the genetic modification to modulate the expression of the one or more endogenous mogroside synthesis pathway genes comprises insertion of one or more functional promoters into the genome of the plant cell or plant part, wherein following said insertion, the one or more functional promoters are operably linked to said one or more mogroside synthesis pathway genes. In certain embodiments of the plant or plant part, the one or more functional promoters are homologous promoters. In certain embodiments of the plant or plant part, the one or more functional promoters are heterologous promoters.

In some such embodiments of the plant or plant part, the promoter is a cassava vein mosaic virus (CSVMV) promoter. In certain embodiments of the plant or plant part, the CSVMV promoter is operably linked to a gene encoding CDS, CYP87D18, EPH3, SQE, UGT720, or UGT94.

In some embodiments of the plant or plant part, the one or more mogrosides is selected from mogrol, mogroside I-Al, mogroside I-El, mogroside IIA, mogroside IIB, mogroside IIE, 7-oxomogroside IIE, 11-oxomogroside Al, mogroside III, mogroside III Al, mogroside III A2, mogroside IIIx, 11-deoxymogroside III, mogroside IV, mogroside IV-A, 11-oxomogroside IV-A, siamenoside I, mogroside V, 7-oxomogroside V, 11-oxomogroside V, mogroside VI, and mogroside VII. In certain embodiments of the plant or plant part, the one or more mogrosides is mogrol. In certain embodiments of the plant or plant part, the one or more mogrosides is mogroside I-Al. In certain embodiments of the plant or plant part, the one or more mogrosides is mogroside I-El. In certain embodiments of the plant or plant part, the one or more mogrosides is mogroside IIA. In certain embodiments of the plant or plant part, the one or more mogrosides is mogroside IIB. In certain embodiments of the plant or plant part, the one or more mogrosides is mogroside IIE. In certain embodiments of the plant or plant part, the one or more mogrosides is 7-oxomogroside IIE. In certain embodiments of the plant or plant part, the one or more mogrosides is 11-oxomogroside Al. In certain embodiments of the plant or plant part, the one or more mogrosides is mogroside III. In certain embodiments of the plant or plant part, the one or more mogrosides is mogroside III Al. In certain embodiments of the plant or plant part, the one or more mogrosides is mogroside III A2. In certain embodiments of the plant or plant part, the one or more mogrosides is mogroside Mx. In certain embodiments of the plant or plant part, the one or more mogrosides is 11-deoxymogroside III. In certain embodiments of the plant or plant part, the one or more mogrosides is mogroside IV. In certain embodiments of the plant or plant part, the one or more mogrosides is mogroside IV-A. In certain embodiments of the plant or plant part, the one or more mogrosides is 11-oxomogroside IV-A. In certain embodiments of the plant or plant part, the one or more mogrosides is siamenoside I. In certain embodiments of the plant or plant part, the one or more mogrosides is mogroside V. In certain embodiments of the plant or plant part, the one or more mogrosides is 7-oxomogroside V. In certain embodiments of the plant or plant part, the one or more mogrosides is 11-oxomogroside V. In certain embodiments of the plant or plant part, the one or more mogrosides is mogroside VI. In certain embodiments of the plant or plant part, the one or more mogrosides is mogroside VII.

In some embodiments of the plant or plant part, the expression of the one or more endogenous mogroside synthesis pathway genes is increased compared to a control plant or plant part.

In some embodiments of the plant or plant part, the plant is a Cucurbitaceae family plant. In some such embodiments of the plant or plant part, the plant is a watermelon plant or cucumber plant. In certain embodiments of the plant or plant part, the plant is a Charleston Gray variety watermelon plant.

In another aspect, the invention provides an extract from a plant or plant part described hereinabove.

In some embodiments of the extract, the extract comprises one or more mogrosides. In some such embodiments of the extract, the one or more mogrosides is selected from mogrol, mogroside I-Al, mogroside I-El, mogroside IIA, mogroside IIB, mogroside TIE, 7-oxomogroside IIE, 11-oxomogroside Al, mogroside III, mogroside III A1, mogroside III A2, mogroside IIIx, 11-deoxymogroside III, mogroside IV, mogroside IV-A, 11-oxomogroside IV-A, siamenoside I, mogroside V, 7-oxomogroside V, 11-oxomogroside V, mogroside VI, and mogroside VII. In certain embodiments of the extract, the one or more mogrosides is mogrol. In certain embodiments of the extract, the one or more mogrosides is mogroside I-Al. In certain embodiments of the extract, the one or more mogrosides is mogroside I-El. In certain embodiments of the extract, the one or more mogrosides is mogroside IIA. In certain embodiments of the extract, the one or more mogrosides is mogroside IIB. In certain embodiments of the extract, the one or more mogrosides is mogroside IIE. In certain embodiments of the extract, the one or more mogrosides is 7-oxomogroside IIE. In certain embodiments of the extract, the one or more mogrosides is 11-oxomogroside A1. In certain embodiments of the extract, the one or more mogrosides is mogroside III. In certain embodiments of the extract, the one or more mogrosides is mogroside III A1. In certain embodiments of the extract, the one or more mogrosides is mogroside III A2. In certain embodiments of the extract, the one or more mogrosides is mogroside IIIx. In certain embodiments of the extract, the one or more mogrosides is 11-deoxymogroside III. In certain embodiments of the extract, the one or more mogrosides is mogroside IV. In certain embodiments of the extract, the one or more mogrosides is mogroside IV-A. In certain embodiments of the extract, the one or more mogrosides is 11-oxomogroside IV-A. In certain embodiments of the extract, the one or more mogrosides is siamenoside I. In specific embodiments of the extract, the one or more mogrosides is mogroside V. In certain embodiments of the extract, the one or more mogrosides is 7-oxomogroside V. In certain embodiments of the extract, the one or more mogrosides is 11-oxomogroside V. In certain embodiments of the extract, the one or more mogrosides is mogroside VI. In certain embodiments of the extract, the one or more mogrosides is mogroside VII.

In some embodiments of the extract, the extract has a sweetness index of 200-450×compared to cane sugar. In some embodiments of the extract, the extract has a glycemic index (GI) of 5 or less. In some such embodiments of the extract, the extract has a GI of 1 or less. In certain embodiments of the extract, the extract has a GI of 0.

In some embodiments of the plant part, the plant part has a sweetness index of 200-450× compared to cane sugar. In some embodiments of the plant part, the plant part has a glycemic index (GI) of 5 or less. In certain embodiments of the plant part, the plant part has a GI of 1 or less. In certain embodiments of the plant part, the plant part has a GI of 0. In some embodiments of the plant part, the plant part is pulp. In some embodiments of the plant part, the plant part is juice.

In another aspect, the invention provides a seed from a plant described hereinabove, wherein the seed comprises the genetic modification to modulate the expression of the one or more endogenous mogroside synthesis pathway genes. In another aspect, the invention provides a plant concentrate of a plant or plant part described hereinabove.

In some embodiments of the plant concentrate, the plant concentrate comprises an increased amount of one or more mogrosides compared to plant concentrate produced from a control plant. In some such embodiments of the plant concentrate, the one or more mogrosides is selected from mogrol, mogroside I-Al, mogroside I-El, mogroside IIA, mogroside IIB, mogroside IIE, 7-oxomogroside IIE, 11-oxomogroside A1, mogroside III, mogroside III A1, mogroside III A2, mogroside Mx, 11-deoxymogroside III, mogroside IV, mogroside IV-A, 11-oxomogroside IV-A, siamenoside I, mogroside V, 7-oxomogroside V, 11-oxomogroside V, mogroside VI, and mogroside VII. In certain embodiments of the plant concentrate, the one or more mogrosides is mogrol. In certain embodiments of the plant concentrate, the one or more mogrosides is mogroside I-Al. In certain embodiments of the plant concentrate, the one or more mogrosides is mogroside I-El. In certain embodiments of the plant concentrate, the one or more mogrosides is mogroside IIA. In certain embodiments of the plant concentrate, the one or more mogrosides is mogroside IIB. In certain embodiments of the plant concentrate, the one or more mogrosides is mogroside IIE. In certain embodiments of the plant concentrate, the one or more mogrosides is 7-oxomogroside IIE. In certain embodiments of the plant concentrate, the one or more mogrosides is 11-oxomogroside A1. In certain embodiments of the plant concentrate, the one or more mogrosides is mogroside III. In certain embodiments of the plant concentrate, the one or more mogrosides is mogroside III A1. In certain embodiments of the plant concentrate, the one or more mogrosides is mogroside III A2. In certain embodiments of the plant concentrate, the one or more mogrosides is mogroside Mx. In certain embodiments of the plant concentrate, the one or more mogrosides is 11-deoxymogroside III. In certain embodiments of the plant concentrate, the one or more mogrosides is mogroside IV. In certain embodiments of the plant concentrate, the one or more mogrosides is mogroside IV-A. In certain embodiments of the plant concentrate, the one or more mogrosides is 11-oxomogroside IV-A. In certain embodiments of the plant concentrate, the one or more mogrosides is siamenoside I. In certain embodiments of the plant concentrate, the one or more mogrosides is mogroside V. In certain embodiments of the plant concentrate, the one or more mogrosides is 7-oxomogroside V. In certain embodiments of the plant concentrate, the one or more mogrosides is 11-oxomogroside V.

In certain embodiments of the plant concentrate, the one or more mogrosides is mogroside VI. In certain embodiments of the plant concentrate, the one or more mogrosides is mogroside VII.

In some embodiments of the plant concentrate, the plant concentrate has a sweetness index of 200-450×compared to cane sugar. In some embodiments of the plant concentrate, the plant concentrate has a glycemic index (GI) of 5 or less. In some such embodiments of the plant concentrate, the plant concentrate has a GI of 1 or less. In certain embodiments of the plant concentrate, the plant concentrate has a GI of 0.

In another aspect, the invention provides a plant powder of a plant or plant part described hereinabove. In some embodiments of the plant powder, the plant powder comprises an increased amount of one or more mogrosides compared to a plant powder produced from a control plant. In some such embodiments of the plant powder, the one or more mogrosides is selected from mogrol, mogroside I-Al, mogroside I-El, mogroside IIA, mogroside IIB, mogroside IIE, 7-oxomogroside IIE, 11-oxomogroside A1, mogroside III, mogroside III A1, mogroside III A2, mogroside IIIx, 11-deoxymogroside III, mogroside IV, mogroside IV-A, 11-oxomogroside IV-A, siamenoside I, mogroside V, 7-oxomogroside V, 11-oxomogroside V, mogroside VI, and mogroside VII. In certain embodiments of the plant powder, the one or more mogrosides is mogrol. In certain embodiments of the plant powder, the one or more mogrosides is mogroside I-Al. In certain embodiments of the plant powder, the one or more mogrosides is mogroside I-El. In certain embodiments of the plant powder, the one or more mogrosides is mogroside IIA. In certain embodiments of the plant powder, the one or more mogrosides is mogroside IIB. In certain embodiments of the plant powder, the one or more mogrosides is mogroside IIE. In certain embodiments of the plant powder, the one or more mogrosides is 7-oxomogroside IIE. In certain embodiments of the plant powder, the one or more mogrosides is 11-oxomogroside A1. In certain embodiments of the plant powder, the one or more mogrosides is mogroside III. In certain embodiments of the plant powder, the one or more mogrosides is mogroside III A1. In certain embodiments of the plant powder, the one or more mogrosides is mogroside III A2. In certain embodiments of the plant powder, the one or more mogrosides is mogroside IIIx. In certain embodiments of the plant powder, the one or more mogrosides is 11-deoxymogroside III. In certain embodiments of the plant powder, the one or more mogrosides is mogroside IV. In certain embodiments of the plant powder, the one or more mogrosides is mogroside IV-A. In certain embodiments of the plant powder, the one or more mogrosides is 11-oxomogroside IV-A. In certain embodiments of the plant powder, the one or more mogrosides is siamenoside I. In certain embodiments of the plant powder, the one or more mogrosides is mogroside V. In certain embodiments of the plant powder, the one or more mogrosides is 7-oxomogroside V. In certain embodiments of the plant powder, the one or more mogrosides is 11-oxomogroside V. In certain embodiments of the plant powder, the one or more mogrosides is mogroside VI. In certain embodiments of the plant powder, the one or more mogrosides is mogroside VII.

In some embodiments of the plant powder, the plant powder has a sweetness index of 200-450×compared to cane sugar. In some embodiments of the plant powder, the plant powder has a glycemic index (GI) of 5 or less. In some such embodiments of the plant powder, the plant powder has a GI of 1 or less. In certain embodiments of the plant powder, the plant powder has a GI of 0. In some embodiments of the plant powder, the plant powder is a formulated plant powder.

In another aspect, the invention provides a plant biomass of a plant described hereinabove. In some embodiments of the plant biomass, the plant biomass comprises an increased amount of one or more mogrosides compared to a plant biomass produced from a control plant. In some such embodiments of the plant biomass, the one or more mogrosides is selected from mogrol, mogroside I-Al, mogroside I-El, mogroside IIA, mogroside IIB, mogroside IIE, 7-oxomogroside IIE, 11-oxomogroside A1, mogroside III, mogroside III A1, mogroside III A2, mogroside Mx, 11-deoxymogroside III, mogroside IV, mogroside IV-A, 11-oxomogroside IV-A, siamenoside I, mogroside V, 7-oxomogroside V, 11-oxomogroside V, mogroside VI, and mogroside VII. In certain embodiments of the plant biomass, the one or more mogrosides is mogrol. In certain embodiments of the plant biomass, the one embodiments of the plant biomass, the one embodiments of the plant biomass, the one embodiments of the plant biomass, the one embodiments of the plant biomass, the one embodiments of the plant biomass, the one embodiments of the plant biomass, the one embodiments of the plant biomass, the one embodiments of the plant biomass, the one embodiments of the plant biomass, the one embodiments of the plant biomass, the one embodiments of the plant biomass, the one or more mogrosides is mogroside I-Al. In certain or more mogrosides is mogroside I-El. In certain or more mogrosides is mogroside IIA. In certain or more mogrosides is mogroside IIB. In certain or more mogrosides is mogroside IIE. In certain or more mogrosides is 7-oxomogroside IIE. In certain or more mogrosides is 11-oxomogroside A1. In certain or more mogrosides is mogroside III. In certain or more mogrosides is mogroside III A1. In certain or more mogrosides is mogroside III A2. In certain or more mogrosides is mogroside IIIx. In certain or more mogrosides is 11-deoxymogroside III. In certain embodiments of the plant biomass, the one or more mogrosides is mogroside IV. In certain embodiments of the plant biomass, the one or more mogrosides is mogroside IV-A. In certain embodiments of the plant biomass, the one or more mogrosides is 11-oxomogroside IV-A. In certain embodiments of the plant biomass, the one or more mogrosides is siamenoside I. In certain embodiments of the plant biomass, the one or more mogrosides is mogroside V. In certain embodiments of the plant biomass, the one or more mogrosides is 7-oxomogroside V. In certain embodiments of the plant biomass, the one or more mogrosides is 11-oxomogroside V. In certain embodiments of the plant biomass, the one or more mogrosides is mogroside VI. In certain embodiments of the plant biomass, the one or more mogrosides is mogroside VII.

In some embodiments of the plant biomass, the plant biomass has a sweetness index of 200-450×compared to cane sugar. In some embodiments of the plant biomass, the plant biomass has a glycemic index (GI) of 5 or less. In some such embodiments of the plant biomass, the plant biomass has a GI of 1 or less. In certain embodiments of the plant biomass, the plant biomass has a GI of 0.

In some embodiments of the plant biomass, the plant biomass is a dried biomass. In some such embodiments of the plant biomass, the dried biomass is crushed and/or powdered biomass.

In another aspect, the invention provides a progeny plant of a plant described hereinabove, wherein the expression of one or more endogenous mogroside synthesis pathway genes is modulated in the progeny plant. In another aspect, the invention provides a seed from such a progeny plant, wherein the expression of the one or more endogenous mogroside synthesis pathway genes is modulated in said seed from said progeny plant.

In another aspect, the invention provides a method for producing a progeny plant having a genetic modification to modulate expression of one or more endogenous mogroside synthesis pathway genes, by crossing a plant described hereinabove with a plant that has not been genetically-modified to modulate the expression of an endogenous mogroside synthesis pathway genes in order to produce a progeny plant having modulated expression of one or more endogenous mogroside synthesis pathway genes compared to a control plant.

In some embodiments of the method, the progeny plant comprises increased levels of at least one mogroside when compared to a control plant.

In some embodiments of the method, the method comprises growing seed from the plant following said crossing step and regenerating said progeny plant from said seed.

In one aspect, an expression construct is provided comprising a promoter and a leader sequence between a pair of nuclease recognition sites, and further comprising a homology arm on each side, wherein the promoter is operably linked to a sequence encoding a polypeptide having at least 80% sequence identity to the amino acid sequence set forth in any one of SEQ ID NOs: 1-20, such as an endogenous mogroside synthesis pathway gene. In specific embodiments, the expression construct comprises a CsVMV promoter and a SynJ 5′ leader sequence between a pair of engineered meganuclease recognition sites, and further comprising a homology arm on each side, wherein the promoter is operably linked to a sequence encoding a polypeptide having at least 80% sequence identity to the amino acid sequence set forth in any one of SEQ ID NOs: 1-20. In specific embodiments, the promoter is a homologous promoter or a heterologous promoter. In some embodiments, the promoter is a heterologous promoter from a watermelon plant or other plant of the Cucurbitaceae family.

### BRIEF DESCRIPTION OF THE DRAWING

FIG. 1 is a schematic representation of the mogroside synthesis pathway.

FIG. 2 is a schematic of an expression construct containing a cassava vein mosaic virus (CSVMV) promoter operably linked to a mogroside gene homolog in watermelon.

FIGS. 3A-3B provide schematic representations of repair templates, each containing a CsVMV promoter and a SynJ 5′ leader sequence inserted between a pair of ARCUS nuclease cleavage sites, as well as 150 bp of homology arms on both sides. FIG. 3A is a schematic representation of a repair template containing a CsVMV promoter and a SynJ 5′ leader sequence inserted between a pair of ARCUS nuclease cleavage sites, U72 3-4 and U72 5-6. FIG. 3B is a schematic representation of a repair template containing a CsVMV promoter and a SynJ 5′ leader sequence inserted between ARCUS nuclease pair, C87 1-2 and C87 3-4.

FIGS. 4A-4B provide graphs showing increased expression of homologs of mogroside synthesis pathway genes following insertion of CsVMV promoter and SynJ 5′ leading sequence, compared to control deliveries, where the DNA repair templates or the ARCUS enzymes were not combined. FIG. 4A provides a graph showing increased expression of a homolog of UGT720 gene following insertion of CsVMV promoter and SynJ 5′ leading sequence, compared to control deliveries, where the DNA repair templates or the ARCUS enzymes were not combined. FIG. 4B provides a graph showing increased expression of a homolog of CYP87 gene following insertion of CsVMV promoter and SynJ 5′ leading sequence, compared to control deliveries, where the DNA repair templates or the ARCUS enzymes were not combined.

### DETAILED DESCRIPTION OF THE INVENTION

### 1.1 References and Definitions

The patent and scientific literature referred to herein establishes knowledge that is available to those of skill in the art. The issued US patents, allowed applications, published foreign applications, and references, including GenBank database sequences, which are cited herein are hereby incorporated by reference to the same extent as if each was specifically and individually indicated to be incorporated by reference.

The present invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference herein in their entirety.

As used herein, “a,” “an,” or “the” can mean one or more than one. For example, “a” cell can mean a single cell or a multiplicity of cells. Further, the term “a gene” may include a plurality of genes, including a group of several genes.

As used herein, unless specifically indicated otherwise, the word “or” is used in the inclusive sense of “and/or” and not the exclusive sense of “either/or.”

The term “about” or “approximately” usually means within 5%, or more preferably within 1%, of a given value or range.

The terms “comprises”, “comprising”, “includes”, “including”, “having” and their conjugates mean “including but not limited to”.

Various embodiments of this disclosure may be presented in a range format. It should be noted that whenever a value or range of values of a parameter are recited, it is intended that values and ranges intermediate to the recited values are also part of this disclosure. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1-10 should be considered to have specifically disclosed subranges such as from 1 to 3, from 1 to 4, from 1 to 5, from 1 to 6, from 1 to 7, from 1 to 8, from 1 to 9, from 2 to 4, from 2 to 6, from 2 to 8, from 2 to 10, from 3 to 6, etc., as well as individual numbers within that range, for example, 1, 2, 3, 4, 5, 6, 7, 8, 9 and 10. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “gene” refers to a functional nucleic acid unit encoding a protein, polypeptide, or peptide. As will be understood by those in the art, this functional term includes genomic sequences, cDNA sequences, and smaller engineered gene segments that express, or may be adapted to express proteins, polypeptides, domains, peptides, fusion proteins, and mutants.

As used herein, the term “genetically-modified” refers to a cell or organism in which, or in an ancestor of which, a genomic DNA sequence has been deliberately modified by recombinant technology. As used herein, the term “genetically-modified” encompasses the terms “recombinant” or “transgenic.”

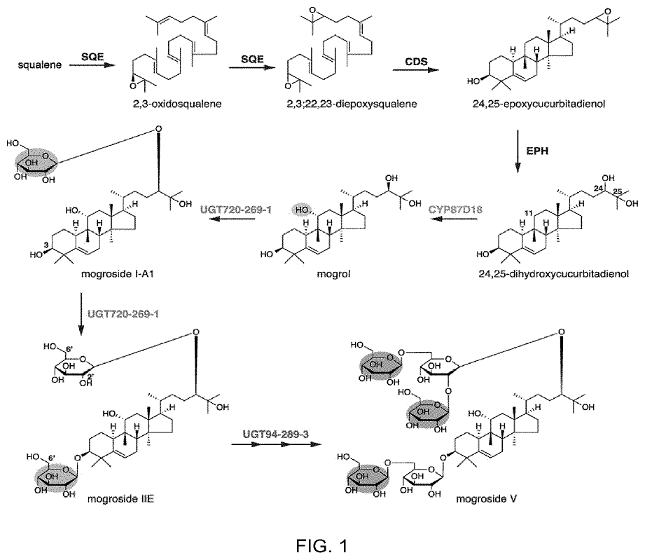

As used interchangeably herein, the term “mogroside synthesis pathway gene” or “mogroside pathway gene” refers to any gene that encodes an enzyme involved in the mogroside synthesis pathway (e.g., an enzyme catalyzing one or more reactions of the mogroside synthesis pathway, as described in FIG. 1), or an active variant or homolog of that gene. In some embodiments, a mogroside synthesis pathway gene is a gene encoding cucurbitadienol synthase (CDS) (SEQ ID NO: 15) or a gene encoding a polypeptide sequence having at least 75% sequence identity thereto; a gene encoding cytochrome P450 enzyme 87D18 (CYP87D18) (SEQ ID NO: 16) or a gene encoding a polypeptide sequence having at least 75% sequence identity thereto; a gene encoding epoxide hydrolase 3 (EPH3) (SEQ ID NO: 17) or a gene encoding a polypeptide sequence having at least 75% sequence identity thereto; a gene encoding squalene epoxidase 1 (SQE1) (SEQ ID NO: 18) or a gene encoding a polypeptide sequence having at least 75% sequence identity thereto; a gene encoding UDP glycosyltransferase 720 (UGT720) (SEQ ID NO: 19) or a gene encoding a polypeptide sequence having at least 75% sequence identity thereto; and/or a gene encoding UDP glycosyltransferase 94 (UGT94) (SEQ ID NO: 20) or a gene encoding a polypeptide sequence having at least 75% sequence identity thereto. In some embodiments, a mogroside synthesis pathway gene is a homolog (e.g., homolog in a Cucurbitaceae family plant, such as a watermelon (e.g., a Charleston Gray variety watermelon) plant or a cucumber plant) of a gene encoding CDS, CYP87D18, EPH3, SQE1, UGT720, and/or UGT94. For example, a mogroside synthesis pathway gene can be one or more homolog of a gene encoding CDS; one or more homolog of a gene encoding CYP87D18; one or more homolog of a gene encoding EPH3; one or more homolog of a gene encoding SQE1; one or more homolog of a gene encoding UGT720; and/or one or more homolog of a gene encoding UGT94. In particular embodiments, a mogroside synthesis pathway gene is a gene encoding a polypeptide sequence having at least 75% sequence identity to the amino acid sequence set forth in any one of SEQ ID NOs: 1-14 and 21.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

As used herein, the term “polypeptide” refers to a linear organic polymer containing a large number of amino-acid residues bonded together by peptide bonds in a chain, forming part of (or the whole of) a protein molecule. The amino acid sequence of the polypeptide refers to the linear consecutive arrangement of the amino acids comprising the polypeptide, or a portion thereof.

As used herein the term “polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

The term “isolated” refers to at least partially separated from the natural environment e.g., from a plant cell.

As used herein, the term “expression” or “expressing” refers to the transcription and/or translation of a particular nucleotide sequence driven by a promoter. As used herein, the terms “exogenous” or “heterologous” in reference to a nucleotide sequence or amino acid sequence are intended to mean a sequence that is purely synthetic, that originates from a foreign species, or, if from the same species, is substantially modified from its native form in composition and/or genomic locus by deliberate human intervention. Thus, a heterologous nucleic acid sequence may not be naturally expressed within the plant (e.g., a nucleic acid sequence from a different species) or may have altered expression when compared to the corresponding wild type plant. An exogenous polynucleotide may be introduced into the plant in a stable or transient manner, so as to produce a ribonucleic acid (RNA) molecule and/or a polypeptide molecule. It should be noted that the exogenous polynucleotide may comprise a nucleic acid sequence which is identical or partially homologous to an endogenous nucleic acid sequence of the plant.

As used herein, the term “endogenous” in reference to a gene or nucleotide sequence or protein is intended to mean a gene or nucleotide sequence or protein that is naturally comprised within or expressed by a cell. For example, in order to increase the expression of mogrosides in a cell of a plant, the expression of one or more endogenous mogroside synthesis pathway genes in the cell (i.e., mogroside synthesis pathway genes naturally comprised within or expressed by the genome of the cell) can be modulated by the methods of the present disclosure. Endogenous genes can include genes that naturally occur in the cell of a plant, but that have been modified in the genome of the cell without insertion or replacement of a heterologous gene that is from another plant species or another location within the genome of the modified cell.

“Homolog” or “homologous sequence” may refer to both orthologous and paralogous sequences. Paralogous sequence relates to gene-duplications within the genome of a species. Orthologous sequence relates to homologous genes in different organisms due to ancestral relationship. Thus, orthologs are evolutionary counterparts derived from a single ancestral gene in the last common ancestor of given two species and therefore have great likelihood of having the same function. One option to identify homologs (e.g., orthologs) in monocot plant species is by performing a reciprocal BLAST search. This may be done by a first blast involving blasting the sequence-of-interest against any sequence database, such as the publicly available NCBI database which may be found at: ncbi.nlm.nih.gov. If orthologs in rice were sought, the sequence-of-interest would be blasted against, for example, the 28,469 full-length cDNA clones from Oryza saliva Nipponbare available at NCBI. The blast results may be filtered. The full-length sequences of either the filtered results or the non-filtered results are then blasted back (second blast) against the sequences of the organism from which the sequence-of-interest is derived. The results of the first and second blasts are then compared. An ortholog is identified when the sequence resulting in the highest score (best hit) in the first blast identifies in the second blast the query sequence (the original sequence-of-interest) as the best hit. Using the same rational a paralog (homolog to a gene in the same organism) is found. In case of large sequence families, the ClustalW program may be used [ebi.ac.uk/Tools/clustalw2/index.html], followed by a neighbor joining tree (wikipedia.org/wiki/Neighbor-joining) which helps visualizing the clustering.

In some embodiments, the term “homolog” as used herein, refers to functional homologs of genes (e.g., functional homologs of mogroside synthesis pathway genes). A functional homolog is a gene encoding a polypeptide that has sequence similarity to a polypeptide encoded by a reference gene, and the polypeptide encoded by the homolog carries out one or more of the biochemical or physiological function(s) of the polypeptide encoded by the reference gene. Thus, functional homologs of mogroside synthesis pathway genes described herein are genes encoding polypeptides that have sequence similarity to the mogroside synthesis pathway enzymes encoded by the reference gene, and which are capable of catalyzing the same step or part of a step of the mogroside synthesis pathway as the reference enzyme. In general, it is preferred that functional homologs and/or polypeptides encoded by functional homologs share at least some degree of sequence identity with the reference gene or polypeptide encoded by the reference gene.

Homology (e.g., percent homology, sequence identity+sequence similarity) can be determined using any homology comparison software computing a pairwise sequence alignment.

As used herein, “sequence identity,” “identity,” “percent identity,” “percentage similarity,” “sequence similarity” and the like refer to a measure of the degree of similarity of two sequences based upon an alignment of the sequences that maximizes similarity between aligned amino acid residues or nucleotides, and which is a function of the number of identical or similar residues or nucleotides, the number of total residues or nucleotides, and the presence and length of gaps in the sequence alignment. A variety of algorithms and computer programs are available for determining sequence similarity using standard parameters. As used herein, sequence similarity is measured using the BLASTp program for amino acid sequences and the BLASTn program for nucleic acid sequences, both of which are available through the National Center for Biotechnology Information (www.ncbi.nlm.nih.gov/), and are described in, for example, Altschul et al. (1990), J. Mol. Biol. 215:403-410; Gish and States (1993), Nature Genet. 3:266-272; Madden et al. (1996), Meth. Enzymo 1.266:131-141; Altschul et al. (1997), Nucleic Acids Res. 25:3389-3402); Zhang et al. (2000), J. Comput. Biol. 7(1-2):203-14. As used herein, percent similarity of two amino acid sequences is the score based upon the following parameters for the BLASTp algorithm: word size=3; gap opening penalty=−11; gap extension penalty=−1; and scoring matrix=BLOSUM62. As used herein, percent similarity of two nucleic acid sequences is the score based upon the following parameters for the BLASTn algorithm: word size=11; gap opening penalty=−5; gap extension penalty=−2; match reward=1; and mismatch penalty=−3. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff J G. (Proc Natl Acad Sci 89:10915-9 (1992)).

Identity (e.g., percent homology) can be determined using any homology comparison software, including for example, the BlastN software of the National Center of Biotechnology Information (NCB I) such as by using default parameters.

According to some embodiments, the identity is a global identity, i.e., an identity over the entire amino acid or nucleic acid sequences of the invention and not over portions thereof.

According to some embodiments, the term “homology” or “homologous” refers to identity of two or more nucleic acid sequences; or identity of two or more amino acid sequences; or the identity of an amino acid sequence to one or more nucleic acid sequence.

According to some embodiments, the homology is a global homology, e.g., a homology over the entire amino acid or nucleic acid sequences of the invention and not over portions thereof.

The degree of homology or identity between two or more sequences can be determined using various known sequence comparison tools which are described in WO2014/102774.

As used herein, the term “recombinant DNA construct,” “recombinant construct,” “expression cassette,” “expression construct,” “chimeric construct,” “construct,” and “recombinant DNA fragment” are used interchangeably herein and are single or double-stranded polynucleotides. A recombinant construct comprises an artificial combination of nucleic acid fragments, including, without limitation, regulatory and coding sequences that are not found together in nature. For example, a recombinant DNA construct may comprise regulatory sequences and coding sequences that are derived from different sources, or regulatory sequences and coding sequences derived from the same source and arranged in a manner different than that found in nature. Such a construct may be used by itself or may be used in conjunction with a vector.

An expression cassette can permit transcription of a particular polynucleotide sequence in a host cell (e.g., a plant cell). An expression cassette may be part of a plasmid, viral genome, or nucleic acid fragment. Typically, an expression cassette includes a polynucleotide to be transcribed, operably linked to a promoter. Other elements that may be present in an expression cassette include those that enhance transcription (e.g., enhancers) and terminate transcription (e.g., terminators), as well as those that confer certain binding affinity or antigenicity to the recombinant protein produced from the expression cassette. For example, an expression construct described herein may contain a promoter sequence operably linked to a mogroside synthesis pathway gene, wherein the promoter may direct (e.g., modulate, such as increase) the expression of the mogroside synthesis pathway gene in a host cell, such as a cell in a plant (e.g., a Cucurbitaceae family plant, such as a watermelon (e.g., a Charleston Gray variety watermelon) plant or a cucumber plant).

As used herein, the term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue-specific manner.

As used herein, the term “operably linked” is intended to mean a functional linkage between two or more elements. For example, an operable linkage between a nucleic acid sequence encoding a protein as disclosed herein and a regulatory sequence (e.g., a promoter) is a functional link that allows for expression of the nucleic acid sequence encoding the protein. Operably linked elements may be contiguous or non-contiguous. When used to refer to the joining of two protein coding regions, by operably linked is intended that the coding regions are in the same reading frame.

As used herein with respect to a parameter, the term “modulation” or “modulating” refers to a detectable positive or negative change in the parameter from a comparison control, e.g., an established normal or reference level of the parameter, or an established standard control. For example, as used herein, a method for modulating gene expression refers to a method disclosed herein that may elicit detectable positive or negative change in expression level of a gene (e.g., change in expression level of a gene in a cell, such as a plant cell) compared to a normal or reference expression level of the gene (e.g., expression level of the gene in the cell before the cell was subject to the said method, or a cell that has not been subject to the said method). Modulation of a parameter (e.g., modulation of gene expression) may refer to increase or decrease of the parameter (e.g., increase or decrease of gene expression). For example, a method for modulating gene expression may elicit detectable (e.g., at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more) increase or positive change in expression level of a gene (e.g., expression level of a gene in a cell, such as a plant cell) compared to a normal or reference expression level of the gene (e.g., expression level of the gene in the cell before the cell was subject to the said method, or a cell that has not been subject to the said method). Alternatively, a method for modulating gene expression may elicit detectable (e.g., at least about 5%, 10%, 15%, 20%, 25%, 30%, 35%, 40%, 45%, 50%, 55%, 60%, 65%, 70%, 75%, 80%, 85%, 90%, 95%, 97%, 99%, or more) decrease, reduction or negative change in expression level of a gene (e.g., expression level of a gene in a cell, such as a plant cell) compared to a normal or reference expression level of the gene (e.g., expression level of the gene in the cell before the cell was subject to the said method, or a cell that has not been subject to the said method).

As used herein with respect to a parameter, the term “increased” or “increasing” or “increase” refers to a detectable (e.g., at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more) positive change in the parameter from a comparison control, e.g., an established normal or reference level of the parameter, or an established standard control. For example, increased production of mogrosides in a plant may indicate detectable (e.g., at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more) increase or positive change in production of mogrosides in a plant compared to a control plant. Similarly, increased level of mogrosides in a plant part (e.g., juice, pulp, etc.) may indicate detectable (e.g., at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more) increase or positive change in level of the mogrosides in a part (e.g., juice, pulp, etc.) of a plant compared to a corresponding control plant part.

As used herein, a “control plant” or “control plant part” or “control cell” refers to a plant or plant part or cell that has not been subject to the methods and compositions described herein, for example, a plant or plant part or cell that has not been subject to the methods and compositions described herein for modulation of expression of one or more mogroside synthesis pathway genes. In some embodiments, a control plant or control plant part or control cell refers to a plant or a plant part or cell that does not have genes (e.g., mogroside synthesis pathway genes) with modulated expression. In particular, as used herein, a “control plant” or “control plant part” or “control cell” may refer to a plant or plant part or cell without the modification (e.g., by methods and compositions described herein) to modulate the expression of one or more endogenous mogroside synthesis pathway genes. For example, a control plant or control plant part or control cell may refer to a plant or plant part or cell that does not have mogroside synthesis pathway genes (e.g., endogenous mogroside synthesis pathway genes) with modulated expression or does not have the same mogroside synthesis pathway gene with modulated expression as the test (e.g., genetically-modified or transgenic) plant or plant part or cell of the present disclosure.

As used herein, the term “plant” includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, pulp, juice, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides. Further provided is a processed plant product (e.g., extract) or byproduct that retains the sequences disclosed herein, including for example, sugar, sweetener, processed plant biomass, etc. As used herein, the term “pulp” refers to a fleshy part of a plant, such as the fleshy part of a fruit, the fleshy part of a stem, etc. In some embodiments of the present disclosure, pulp refers to the edible, fleshy part of the fruit of a plant (e.g., a Cucurbitaceae family plant, such as a watermelon (e.g., a Charleston Gray variety watermelon) plant or a cucumber plant) described herein.

As used herein, the term “sweetener” refers to natural or artificial substances that provide a sweet taste when added to food and beverages or tasted alone. In some embodiments of the present disclosure, a sweetener is a type of an extract, such as a crystalline extract from a plant (e.g., a Cucurbitaceae family plant, such as a watermelon plant or a cucumber plant) described herein that provides a sweet taste when added to food and beverages or tasted alone. A sweetener described herein may contain one or more mogrosides. For example, in specific embodiments, the sweetener comprises at least one of mogrol, mogroside I-Al, mogroside I-El, mogroside IIA, mogroside IIB, mogroside ITE, 7-oxomogroside TIE, 11-oxomogroside A1, mogroside III, mogroside III A1, mogroside III A2, mogroside IIIx, 11-deoxymogroside III, mogroside IV, mogroside IV-A, 11-oxomogroside IV-A, siamenoside I, mogroside V, 7-oxomogroside V, 11-oxomogroside V, mogroside VI, and/or mogroside VII. Additionally, or alternatively, a sweetener described herein may be one or more mogrosides extracted from a plant described herein modified to have modulated expression of one or more mogroside synthesis pathway genes, or a progeny plant thereof. In specific embodiments, the mogroside extracted from the genetically-modified plant or progeny thereof can undergo processing in order to purify the mogroside, and/or crystallize the mogroside. In this way, a crystalline sweetener comprising the one or more mogroside produced by a genetically-modified plant, or progeny thereof, described herein can be manufactured.

As used herein, “genome editing” or “genome edits” or “modification” or “genetic modification” or “genetically-modifying” or “genetically-modified” or “engineered” or “engineering” or “genetic engineering” refers to any insertion, deletion, or substitution of an amino acid residue in the recombinant sequence relative to a reference sequence (e.g., a wild-type or a native sequence) or can refer to single strand cleavage, double strand cleavage or binding to a nucleic acid molecule in the genome of a cell or outside (e.g., plasmid) of the genome of a cell. “Modification” or “modifying” may indicate any detectable positive or negative effect on a process or on the function of a target, such as a promoter, a transcription factor gene, etc. Modification of a target (e.g., modification of a promoter) may refer to activation or inhibition of the target (e.g., activation or inhibition of the promoter). For example, modification of a promoter may indicate detectable (e.g., at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more) activation or positive effect on the function of the promoter (e.g., function of the promoter in the genome of a cell, such as a plant cell) compared to a normal or reference level (e.g., function of the promoter in the genome of the cell before the cell was subject to the said method, or a cell that has not been subject to the said method). Alternatively, modification of a promoter may indicate detectable (e.g., at least about 5%, 10%, 15%, 20%, 25%, 30%, 35%, 40%, 45%, 50%, 55%, 60%, 65%, 70%, 75%, 80%, 85%, 90%, 95%, 97%, 99%, or more) inhibition or negative effect on the function or activity of the promoter (e.g., function of the promoter in the genome of a cell, such as a plant cell) compared to a normal or reference level (e.g., function of the promoter in the genome of the cell before the cell was subject to the said method, or a cell that has not been subject to the said method). Accordingly, “genetically-modified plant” or “genetically-modified plant part” or “genetically-modified cell” or “genetically-modified plant genome” refers to a plant or plant part or cell or genome that has been subject to one or more modifications described hereinabove. For example, a genetically-modified plant or genetically-modified plant part or genetically-modified cell may refer to a plant or plant part or cell, wherein the genome of the plant or plant part or cell has been genetically-modified to modulate the expression of one or more endogenous mogroside synthesis pathway genes.

As used herein with respect to a test product (e.g., a test food product, such as a sweetener, pulp, or a juice from a plant described herein), the term “glycemic index” or “GP” refers to the relative rise in blood glucose level two hours after consuming that product. A GI value of 0 to 100 may be assigned to a test product (e.g., a test food product, such as a sweetener, pulp, or a juice from a plant described herein), with pure glucose arbitrarily given the value of 100. The GI value of a test product (e.g., a test food product, such as a sweetener, pulp, or a juice from a plant described herein) can be measured by the incremental area under two-hour blood glucose response curve (AUC) following a 12-hour fast and ingestion of the product with a certain quantity of available carbohydrate (usually 50 g). The AUC of the test product is divided by the AUC of the standard (either glucose or white bread, giving two different definitions) and multiplied by 100. The average GI value is calculated from data collected in 10 human subjects. Both the standard and test product must contain an equal amount of available carbohydrate. The result gives a relative ranking for each test product. A test product (e.g., a test food product, such as a sweetener, pulp, or a juice from a plant described herein) is considered to have a low GI if it is 55 or less; high GI if 70 or more; and mid-range GI if it is 56 to 69.

As used herein with respect to a test product (e.g., a test food product, such as a sweetener, pulp, or a juice from a plant described herein), the term “sweetness index” refers to a measure of sweetness in the test product relative to cane sugar. Sweetness index of a test product (e.g., a test food product, such as a sweetener, pulp, or a juice from a plant described herein) relative to cane sugar can be measured by comparing the threshold value of the test product with the threshold value of cane sugar. Threshold value of the test product or cane sugar can be determined by the concentration at which the presence of the test product or the cane sugar in water can be detected (e.g., tasted) by half of a panel of trained tasters. Any sensory analysis method can be used to evaluate the relative sweetness of a mogroside, including but not limited to 2-alternative forced choice (2-AFC) and triangle test.

As used herein, the term “meganuclease” refers to an endonuclease that binds double-stranded DNA at a recognition sequence that is greater than 12 base pairs. In some embodiments, the recognition sequence for a meganuclease of the present disclosure is 22 base pairs. A meganuclease can be an endonuclease that is derived from I-Crel (SEQID NO: 22), and can refer to an engineered variant of I-Crel that has been modified relative to natural I-Crel with respect to, for example, DNA-binding specificity, DNA cleavage activity, DNA-binding affinity, or dimerization properties. Methods for producing such modified variants of I-Crel are known in the art (e.g., WO 2007/047859, incorporated by reference in its entirety). A meganuclease as used herein binds to double-stranded DNA as a heterodimer. A meganuclease may also be a “single-chain meganuclease” in which a pair of DNA-binding domains is joined into a single polypeptide using a peptide linker. The term “homing endonuclease” is synonymous with the term “meganuclease.” Meganucleases of the present disclosure are substantially non-toxic when expressed in the targeted cells as described herein such that cells can be transfected and maintained at 37° C. without observing deleterious effects on cell viability or significant reductions in meganuclease cleavage activity when measured using the methods described herein.

As used herein, the term “single-chain meganuclease” refers to a polypeptide comprising a pair of nuclease subunits joined by a linker. A single-chain meganuclease has the organization: N-terminal subunit —Linker—C-terminal subunit. The two meganuclease subunits will generally be non-identical in amino acid sequence and will bind non-identical DNA sequences. Thus, single-chain meganucleases typically cleave pseudo-palindromic or non-palindromic recognition sequences. A single-chain meganuclease may be referred to as a “single-chain heterodimer” or “single-chain heterodimeric meganuclease” although it is not, in fact, dimeric. For clarity, unless otherwise specified, the term “meganuclease” can refer to a dimeric or single-chain meganuclease.

As used herein, the terms “nuclease” and “endonuclease” are used interchangeably to refer to naturally-occurring or engineered enzymes, which cleave a phosphodiester bond within a polynucleotide chain.

As used herein, the term “compact TALEN” refers to an endonuclease comprising a DNA-binding domain with one or more TAL domain repeats fused in any orientation to any portion of the I-Tevl homing endonuclease or any of the endonucleases listed in Table 2 in U.S. Application No. 20130117869 (which is incorporated by reference in its entirety), including but not limited to Mmel, EndA, Endl, I-Basl, I-TevII, I-TevIII, I-Twol, Mspl, Mval, NucA, and NucM. Compact TALENs do not require dimerization for DNA processing activity, alleviating the need for dual target sites with intervening DNA spacers. In some embodiments, the compact TALEN comprises 16-22 TAL domain repeats.

As used herein, the terms “CRISPR nuclease” or “CRISPR system nuclease” refers to a CRISPR (clustered regularly interspaced short palindromic repeats)-associated (Cas) endonuclease or a variant thereof, such as Cas9, that associates with a guide RNA that directs nucleic acid cleavage by the associated endonuclease by hybridizing to a recognition site in a polynucleotide. In certain embodiments, the CRISPR nuclease is a class 2 CRISPR enzyme. In some of these embodiments, the CRISPR nuclease is a class 2, type II enzyme, such as Cas9. In other embodiments, the CRISPR nuclease is a class 2, type V enzyme, such as Cpfl or Cas12a. The guide RNA comprises a direct repeat and a guide sequence (often referred to as a spacer in the context of an endogenous CRISPR system), which is complementary to the target recognition site. In certain embodiments, the CRISPR system further comprises a tracrRNA (trans-activating CRISPR RNA) that is complementary (fully or partially) to the direct repeat sequence (sometimes referred to as a tracr-mate sequence) present on the guide RNA. In particular embodiments, the CRISPR nuclease can be mutated with respect to a corresponding wild-type enzyme such that the enzyme lacks the ability to cleave one strand of a target polynucleotide, functioning as a nickase, cleaving only a single strand of the target DNA. Non-limiting examples of CRISPR enzymes that function as a nickase include Cas9 enzymes with a DIOA mutation within the RuvC I catalytic domain, or with a H840A, N854A, or N863A mutation. Given a predetermined DNA locus, recognition sequences can be identified using a number of programs known in the art (Kornel Labun; Tessa G. Montague; James A. Gagnon; Summer B. Thyme; Eivind Valen. (2016). CHOPCHOP v2: a web tool for the next generation of CRISPR genome engineering. Nucleic Acids Research; doi:10.1093/nar/gkw398; Tessa G. Montague; Jose M. Cruz; James A. Gagnon; George M. Church; Eivind Valen. (2014).CHOPCHOP: a CRISPR/Cas9 and TALEN web tool for genome editing. Nucleic Acids Res. 42. W401-W407).

As used herein, the term “megaTAL” refers to a single-chain endonuclease comprising a transcription activator-like effector (TALE) DNA binding domain with an engineered, sequence-specific homing endonuclease.

As used herein, the term “TALEN” refers to an endonuclease comprising a DNA-binding domain comprising a plurality of TAL domain repeats fused to a nuclease domain or an active portion thereof from an endonuclease or exonuclease, including but not limited to a restriction endonuclease, homing endonuclease, Si nuclease, mung bean nuclease, pancreatic DNAse I, micrococcal nuclease, and yeast HO endonuclease. See, for example, Christian et al. (2010) Genetics 186:757-761, which is incorporated by reference in its entirety. Nuclease domains useful for the design of TALENs include those from a Type IIs restriction endonuclease, including but not limited to Fokl, FoM, Stsl, Hhal, HindIII, Nod, BbvCI, EcoRI, BgII, and Alwl. Additional Type IIs restriction endonucleases are described in International Publication No. WO 2007/014275, which is incorporated by reference in its entirety. In some embodiments, the nuclease domain of the TALEN is a Fokl nuclease domain or an active portion thereof. TAL domain repeats can be derived from the TALE (transcription activator-like effector) family of proteins used in the infection process by plant pathogens of the Xanthomonas genus. TAL domain repeats are 33-34 amino acid sequences with divergent 12th and 13th amino acids. These two positions, referred to as the repeat variable dipeptide (RVD), are highly variable and show a strong correlation with specific nucleotide recognition. Each base pair in the DNA target sequence is contacted by a single TAL repeat with the specificity resulting from the RVD. In some embodiments, the TALEN comprises 16-22 TAL domain repeats. DNA cleavage by a TALEN requires two DNA recognition regions (i.e., “half-sites”) flanking a nonspecific central region (i.e., the “spacer”). The term “spacer” in reference to a TALEN refers to the nucleic acid sequence that separates the two nucleic acid sequences recognized and bound by each monomer constituting a TALEN. The TAL domain repeats can be native sequences from a naturally-occurring TALE protein or can be redesigned through rational or experimental means to produce a protein that binds to a pre-determined DNA sequence (see, for example, Boch et al. (2009) Science 326(5959):1509-1512 and Moscouand Bogdanove (2009) Science 326(5959):1501, each of which is incorporated by reference in its entirety). See also, U.S. Publication No. 20110145940 and International Publication No. WO 2010/079430 for methods for engineering a TALEN to recognize and bind a specific sequence and examples of RVDs and their corresponding target nucleotides. In some embodiments, each nuclease (e.g., Fokl) monomer can be fused to a TAL effector sequence that recognizes and binds a different DNA sequence, and only when the two recognition sites are in close proximity do the inactive monomers come together to create a functional enzyme. It is understood that the term “TALEN” can refer to a single TALEN protein or, alternatively, a pair of TALEN proteins (i.e., a left TALEN protein and a right TALEN protein) which bind to the upstream and downstream half-sites adjacent to the TALEN spacer sequence and work in concert to generate a cleavage site within the spacer sequence. Given a predetermined DNA locus or spacer sequence, upstream and downstream half-sites can be identified using a number of programs known in the art (Kornel Labun; Tessa G. Montague; James A. Gagnon; Summer B. Thyme; Eivind Valen. (2016). CHOPCHOP v2: a web tool for the next generation of CRISPR genome engineering. Nucleic Acids Research; doi:10.1093/nar/gkw398; Tessa G. Montague; Jose M. Cruz; James A. Gagnon; George M. Church; Eivind Valen. (2014). CHOPCHOP: a CRISPR/Cas9 and TALEN web tool for genome editing. Nucleic Acids Res. 42. W401-W407). It is also understood that a TALEN recognition sequence can be defined as the DNA binding sequence (i.e., half-site) of a single TALEN protein or, alternatively, a DNA sequence comprising the upstream half-site, the spacer sequence, and the downstream half-site.

As used herein, the terms “zinc finger nuclease” or “ZFN” refers to a chimeric protein comprising a zinc finger DNA-binding domain fused to a nuclease domain from an endonuclease or exonuclease, including but not limited to a restriction endonuclease, homing endonuclease, Si nuclease, mung bean nuclease, pancreatic DNAse I, micrococcal nuclease, and yeast HO endonuclease. Nuclease domains useful for the design of zinc finger nucleases include those from a Type Its restriction endonuclease, including but not limited to Fokl, FoM, and Stsl restriction enzyme. Additional Type Its restriction endonucleases are described in International Publication No. WO 2007/014275, which is incorporated by reference in its entirety. The structure of a zinc finger domain is stabilized through coordination of a zinc ion. DNA binding proteins comprising one or more zinc finger domains bind DNA in a sequence-specific manner. The zinc finger domain can be a native sequence or can be redesigned through rational or experimental means to produce a protein which binds to a pre-determined DNA sequence—18 base pairs in length, comprising a pair of nine basepair half-sites separated by 2-10 base pairs. See, for example, U.S. Pat. Nos. 5,789,538, 5,925,523, 6,007,988, 6,013,453, 6,200,759, and International Publication Nos. WO 95/19431, WO 96/06166, WO 98/53057, WO 98/54311, WO 00/27878, WO 01/60970, WO 01/88197, and WO 02/099084, each of which is incorporated by reference in its entirety. By fusing this engineered protein domain to a nuclease domain, such as Fokl nuclease, it is possible to target DNA breaks with genome-level specificity. The selection of target sites, zinc finger proteins and methods for design and construction of zinc finger nucleases are known to those of skill in the art and are described in detail in U.S. Publications Nos. 20030232410, 20050208489, 2005064474, 20050026157, 20060188987 and International Publication No. WO 07/014275, each of which is incorporated by reference in its entirety. In the case of a zinc finger, the DNA binding domains typically recognize an 18-bp recognition sequence comprising a pair of nine basepair “half-sites” separated by a 2-10 basepair “spacer sequence”, and cleavage by the nuclease creates a blunt end or a 5′ overhang of variable length (frequently four base pairs). It is understood that the term “zinc finger nuclease” can refer to a single zinc finger protein or, alternatively, a pair of zinc finger proteins (i.e., a left ZFN protein and a right ZFN protein) that bind to the upstream and downstream half-sites adjacent to the zinc finger nuclease spacer sequence and work in concert to generate a cleavage site within the spacer sequence. Given a predetermined DNA locus or spacer sequence, upstream and downstream half-sites can be identified using a number of programs known in the art (Mandell J G, Barbas C F 3rd. Zinc Finger Tools: custom DNA-binding domains for transcription factors and nucleases. Nucleic Acids Res. 2006 Jul. 1; 34 (Web Server issue):W516-23). It is also understood that a zinc finger nuclease recognition sequence can be defined as the DNA binding sequence (i.e., half-site) of a single zinc finger nuclease protein or, alternatively, a DNA sequence comprising the upstream half-site, the spacer sequence, and the downstream half-site.

As used herein, the term “recognition half-site,” “recognition sequence half-site,” or simply “half-site” means a nucleic acid sequence in a double-stranded DNA molecule that is recognized and bound by a monomer of a homodimeric or heterodimeric meganuclease or by one subunit of a single-chain meganuclease or by one subunit of a single-chain meganuclease, or by a monomer of a TALEN or zinc finger nuclease.

As used herein, the terms “recognition sequence” or “recognition site” or “cleavage site” or “cleavage sequence” refers to a DNA sequence that is bound and cleaved by a nuclease. In the case of a meganuclease, a recognition sequence comprises a pair of inverted, 9 basepair “half sites” which are separated by four base pairs. In the case of a single-chain meganuclease, the N-terminal domain of the protein contacts a first half-site and the C-terminal domain of the protein contacts a second half-site. Cleavage by a meganuclease produces four basepair 3′ overhangs. “Overhangs,” or “sticky ends” are short, single-stranded DNA segments that can be produced by endonuclease cleavage of a double-stranded DNA sequence. In the case of meganucleases and single-chain meganucleases derived from I-Crel, the overhang comprises bases 10-13 of the 22 basepair recognition sequence. In the case of a compact TALEN, the recognition sequence comprises a first CNNNGN sequence that is recognized by the I-Tevl domain, followed by a non-specific spacer 4-16 base pairs in length, followed by a second sequence 16-22 bp in length that is recognized by the TAL-effector domain (this sequence typically has a 5′ T base). Cleavage by a compact TALEN produces two basepair 3′ overhangs. In the case of a CRISPR nuclease, the recognition sequence is the sequence, typically 16-24 base pairs, to which the guide RNA binds to direct cleavage. Full complementarity between the guide sequence and the recognition sequence is not necessarily required to effect cleavage. Cleavage by a CRISPR nuclease can produce blunt ends (such as by a class 2, type II CRISPR nuclease) or overhanging ends (such as by a class 2, type V CRISPR nuclease), depending on the CRISPR nuclease. In those embodiments wherein a Cpfl or Cas12a CRISPR nuclease is utilized, cleavage by the CRISPR complex comprising the same will result in 5′ overhangs and in certain embodiments, 5-nucleotide 5′ overhangs. Each CRISPR nuclease enzyme also requires the recognition of a PAM (protospacer adjacent motif) sequence that is near the recognition sequence complementary to the guide RNA. The precise sequence, length requirements for the PAM, and distance from the target sequence differ depending on the CRISPR nuclease enzyme, but PAMs are typically 2-5 base pair sequences adjacent to the target/recognition sequence. PAM sequences for particular CRISPR nuclease enzymes are known in the art (see, for example, U.S. Pat. No. 8,697,359 and U.S. Publication No. 20160208243, each of which is incorporated by reference in its entirety) and PAM sequences for novel or engineered CRISPR nuclease enzymes can be identified using methods known in the art, such as a PAM depletion assay (see, for example, Karvelis et al. (2017) Methods 121-122:3-8, which is incorporated herein in its entirety). In the case of a zinc finger, the DNA binding domains typically recognize an 18-bp recognition sequence comprising a pair of nine basepair “half-sites” separated by 2-10 base pairs and cleavage by the nuclease creates a blunt end or a 5′ overhang of variable length (frequently four base pairs).

As used herein, the terms “target site” or “target sequence” refers to a region of the chromosomal DNA of a cell comprising a recognition sequence for a nuclease.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments, unless the embodiment is inoperative without those elements.

### 2.1 Principle of the Invention

Mogrosides naturally found in the fruit of the Cucurbitaceae family plant Siraitia grosvenorii (Luo Han Guo or monk fruit) could be a low-calorie alternative to refined sugar if sufficient quantities of the compounds can be produced. However, in planta production of mogrosides by monk fruits is non-scalable due to the difficulties of growing these plants outside of their native habitat. Hence, there is a need to produce mogrosides in widely adapted plant systems.

Described herein are methods for producing mogrosides in plants that can be cultivated with reliable agronomic practices, steady productivity history, and consistent downstream handling and processing practices. Thus, the methods described herein addresses the unmet need in the industry of a scalable, natural and economical production system for mogrosides. Also described herein are plants that are genetically-modified to modulate the expression of one or more endogenous mogroside synthesis pathway genes, resulting in increased levels of one or more mogrosides compared to control plants. Furthermore, provided herein are plant parts (e.g., juice, pulp, seed, fruit, flowers, nectar, embryos, pollen, ovules, leaves, stems, branches, bark, kernels, ears, cobs, husks, stalks, roots, root tips, anthers, etc.), plant concentrate (e.g., whole plant concentrate or plant part concentrate), plant powder (e.g., formulated powder, such as formulated plant part powder), plant biomass (e.g., dried biomass, such as crushed and/or powdered biomass), extract (e.g., sweetener) from such genetically-modified plants, and methods of producing such genetically-modified plants or progeny of such plants. In some embodiments, extract (e.g., sweetener), plant parts (e.g., juice, pulp, seed, fruit, flowers, embryos, pollen, ovules, leaves, branches, kernels, ears, cobs, husks, stalks, roots, root tips, anthers, etc.), plant concentrate (e.g., whole plant concentrate or plant part concentrate), plant biomass (e.g., dried biomass, such as crushed and/or powdered biomass), and/or plant powder (e.g., formulated powder, such as formulated plant part powder) from such plants have increased levels of mogrosides. In certain instances, the plants described herein are Cucurbitaceae family plants, such as Cucurbitaceae family plants that are easy-to-cultivate with reliable agronomic practices. Hence, compositions and methods described herein leverage the genetic similarity between monk fruit and another easy-to-cultivate Cucurbitaceae family plant to reactivate the natural mogroside synthesis pathway in the later, so as to ensure a natural, scalable and yet economic production system for mogrosides. The compositions and methods described herein increase the production of mogrosides by modulating the expression of at least one endogenous mogroside synthesis pathway gene.

In some embodiments, the mogroside described herein is one or more of mogrol, mogroside I-Al, mogroside I-El, mogroside IIA, mogroside IIB, mogroside IIE, 7-oxomogroside IIE, 11-oxomogroside A1, mogroside III, mogroside III A1, mogroside III A2, mogroside Mx, 11-deoxymogroside Ill, mogroside IV, mogroside IV-A, 11-oxomogroside IV-A, siamenoside I, mogroside V, 7-oxomogroside V, 11-oxomogroside V, mogroside VI, and mogroside VII. In particular embodiments, the mogroside described herein is mogrol, mogroside I-Al, mogroside mogroside V, mogroside VI, and/or mogroside VII. In certain embodiments, the mogroside described herein is mogroside V.

### 2.2 Cucurbitaceae Family Plants

In some embodiments, described herein are methods for producing mogrosides in Cucurbitaceae family plants. The Cucurbitaceae family, also called cucurbits or the gourd family, is a plant family that is grown around the tropics and in temperate areas, where Cucurbitaceae family plants with edible fruits were among the earliest cultivated plants. The family Cucurbitaceae ranks among the highest of plant families for number and percentage of species used as human food. According to the Plants Database of the United States Department of Agriculture, the Cucurbitaceae family contains 34 Genera and 127 accepted taxa overall. Genera in the Cucurbitaceae family include the following: Genus Apodanthera Arn.—apodanthera; Genus Benincasa Savi—benincasa; Genus Brandegea Cogn.—starvine; Genus Bryonia L.—bryony; Genus Cayaponia Silva Manso—melonleaf; Genus Citrullus Schrad.—watermelon; Genus Coccinia Wight & Arn.—coccinia; Genus Ctenolepis Benth. & Hook. f.—ctenolepis; Genus Cucumeropsis Naudin—cucumeropsis; Genus CLIC1,1111i,S L.—melon; Genus Cucurbita L. —gourd; Genus Cyclanthera Schrad.—Cyclanthera; Genus Doyerea Gros.—doyeria; Genus Ecballium A. Rich.—squirting cucumber; Genus Echinocyslis Torr. & A. Gray—echinocystis; Genus Echinopepon Naud.—balsam apple; Genus Fevillea L.—fevillea; Genus Hodgsonia Hook. f. & Thomson—hodgsonia; Genus Ibervillea Greene—globeberry; Genus Lagenaria Ser.—lagenaria; Genus Luffa Mill. —luffa; Genus Marah Kellogg—manroot; Genus Melothria L.—melothria; Genus Momordica L.—momordica; Genus Psiguria Neck. ex Arn.—pygmymelon; Genus Sechium P. Br.—sechium; Genus Sicana Naud.—sicana; Genus Sicyos L.—bur cucumber; Genus Sicyosperma A. Gray—sicyosperma; Genus Telfairia Hook.—telfairia; Genus Thladiantha Bunge—thladiantha; Genus Trichosanthes L.—trichosanthes; Genus Tumatnoca Rose—tumamoca; and Genus Zehneria Endl.

In certain embodiments, the Cucurbitaceae family plant described in the instant disclosure is a watermelon plant (e.g., Citrullus lanatus) or a cucumber plant (e.g., CliCffilliS sativus). For example, the methods disclosed herein may be used for producing mogrosides in a watermelon or cucumber plant. In particular embodiments, the Cucurbitaceae family plant described in the instant disclosure is a Charleston Gray variety watermelon plant. For example, the methods disclosed herein may be used for producing mogrosides in a Charleston Gray variety watermelon plant.

In some embodiments, the methods described herein may increase the levels of mogrosides produced in plants (e.g., a Cucurbitaceae family plant) and plant parts by genetically-modifying the genome of cells or parts of the plants to modulate the expression of one or more endogenous mogroside synthesis pathway genes. In certain embodiments, methods of the present disclosure may increase the levels of mogrosides produced in plants (e.g., a Cucurbitaceae family plant) and plant parts by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more, compared to a control plant or plant part. Additionally, or alternatively, methods of the present disclosure may increase the levels of mogrosides produced in plants and plant parts by at least 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more, compared to a control plant or plant part. In particular embodiments, the methods described herein may increase the levels of mogrosides produced in a cell, such as in a cell of a plant (e.g., in a cell of a Cucurbitaceae family plant) or plant part. In specific embodiments, the methods described herein may increase the levels of at least one mogroside in a plant or plant part, such as in a cell of the plant or plant part, by modulating the expression of one or more genes (e.g., endogenous genes) of the mogroside synthesis pathway (e.g., by modulating the expression of one or more mogroside synthesis pathway genes in the plant genome).

In some embodiments, the amount or level of one or more mogrosides in a plant, plant part (e.g., seed, fruit, juice, pulp, etc.), plant extract (e.g., sweetener), plant concentrate, plant powder, and/or plant biomass described herein may be determined by one or more standard methods known in the art. In certain embodiments, the amount or level of one or more mogrosides in a plant, plant part, plant extract, plant concentrate, plant powder, and/or plant biomass described herein may be determined by nuclear magnetic resonance spectroscopy, which is also known as NMR spectroscopy or magnetic resonance spectroscopy (MRS). In additional or alternative embodiments, the amount or level of one or more mogrosides in a plant, plant part, plant extract, plant concentrate, plant powder, and/or plant biomass described herein may be determined by ultraviolet (UV)-absorption spectroscopy or fluorescence spectroscopy. See, for example, Itkin et al. (Proc Natl Acad Sci 113: E7619-E7628 (2016)).

Also described herein are plants (e.g., a Cucurbitaceae family plant) or plant parts, wherein the genome of cells or parts of the plants are genetically-modified to modulate the expression of one or more endogenous mogroside synthesis pathway genes, resulting in increased levels of at least one mogroside. In some embodiments, the level of mogrosides produced in plants and plant parts (e.g., a Cucurbitaceae family plant, such as a watermelon or cucumber plant) of the present disclosure may increase by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more, compared to a control plant or plant part. Additionally, or alternatively, the level of mogrosides produced in plants or plant parts of the present disclosure may increase by at least 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more, compared to a control plant or plant part. In certain embodiments, plants with increased levels of at least one mogroside have modulation (e.g., increase or decrease) in expression of one or more endogenous mogroside synthesis pathway genes. For example, plants with increased levels of at least one mogroside may have increased expression of one or more endogenous mogroside synthesis pathway genes. In particular, described herein are plants and plant parts, wherein the genome of cells or parts of the plants or plant parts are genetically-modified to modulate expression of one or more endogenous mogroside synthesis pathway genes, resulting in the plants having increased levels of at least one mogroside.

Also described herein are methods of producing plants described hereinabove, such as plants having increased levels of at least one mogroside. In certain embodiments, the method involves genetically-modifying the genome of plant cells or plant parts to modulate the expression of one or more endogenous genes of the mogroside synthesis pathway (e.g., modulating the expression of one or more mogroside synthesis pathway genes in the plant genome), and growing a plant from those plant cells or plant parts. In particular embodiments, the method involves genetically-modifying the genome of plant cells or plant parts to modulate the expression of one or more endogenous genes of the mogroside synthesis pathway, and growing a plant from those plant cells or plant parts, thus producing plants, having increased levels of mogrosides.

In some embodiments, the genome of a plant or plant part disclosed herein is genetically-modified to modulate (e.g., increase or decrease) expression of one or more endogenous genes of the mogroside synthesis pathway. In certain embodiments, the genome of a plant or plant part is genetically-modified to modulate (e.g., increase or decrease) the expression of one or more endogenous genes of the mogroside synthesis pathway by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more in a plant or plant part of a plant disclosed herein compared to a control plant or control plant part (e.g., a plant or plant part without the modification to modulate the expression of an endogenous mogroside synthesis pathway gene). In particular embodiments, the expression of one or more endogenous genes of the mogroside synthesis pathway is increased by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more in a plant part of a plant disclosed herein compared to a plant part from a control plant.

Expression of an endogenous mogroside synthesis pathway gene can be measured by any means in the art for measuring gene expression. In some embodiments, expression can be measured by measuring the total amount of mRNA, protein, or any other product of gene expression. For example, gene expression can be measured by a nucleic acid analysis method such as PCR, or RT-PCR, or can be measured by a protein detection method, such as a Western blot analysis.

The present disclosure also describes extracts (e.g., sweetener, antioxidants, alkaloids, etc.) from plants (e.g., a Cucurbitaceae family plant, such as a plant or a cucumber plant) that are disclosed hereinabove, such as plants, wherein the genome of cells or parts of the plants are genetically-modified to modulate expression of one or more endogenous mogroside synthesis pathway genes. In some embodiments, the extract from the plant is a sweetener. For example, the present disclosure may describe a sweetener from a plant that is disclosed hereinabove, such as plants having modulated expression of one or more endogenous mogroside synthesis pathway genes. In some embodiments, a sweetener from a plant disclosed herein has a high sweetness index relative to the sweetness index of extracts from a control plant. The sweetness index of a sweetener from a plant disclosed herein can be measured relative to cane sugar. In particular, sweetness index relative to cane sugar can be measured by comparing threshold value of the sweetener with the threshold value of cane sugar. Threshold value of a sweetener or cane sugar may indicate the concentration at which the presence of the sweetener or the cane sugar in water can be detected by half of a panel of trained tasters. For example, researchers may present each participant of a panel with samples of water to which varying degrees of the sweetener has been added. First, the tasters may be given plain water, and then, samples of water with increasing concentrations of the sweetener (e.g., sweetener from a plant disclosed herein) or cane sugar until the presence of the sweetener or the cane sugar is detected (e.g., tasted) in the water samples by the tasters. Concentration of the sweetener (e.g., a sweetener from a plant disclosed herein) or cane sugar at which half of the panel of tasters can detect a change in the water sample (e.g., a change in taste of the water sample) may be considered as the threshold value of the sweetener or the cane sugar. Threshold value of the sweetener (e.g., sweetener from a plant disclosed herein) can then be compared to threshold value of cane sugar to determine the sweetness index of the sweetener relative to cane sugar.

In some embodiments, a sweetener from a plant disclosed herein has a sweetness index of at least 50×, 100×, 150×, 200×, 250×, 300×, 350×, 400×, 450×, 500×, 550×, 600×, 650×, 700×, 750×, 800×, 850×, 900×, 950×, 1000×, or more compared to cane sugar. For example, a sweetener from a plant disclosed herein can have a sweetness index of about 50-1000×, or more, or about 50-300×, 200-450×, 350-600×, 500-750×, 750-1000×, or more (e.g., about 50×, 100×, 150×, 200×, 250×, 300×, 350×, 400×, 450×, 500×, 550×, 600×, 650×, 700×, 750×, 800×, 850×, 900×, 950×, 1000×, or more, compared to cane sugar.

Alternatively, a sweetener from a plant, which is modified to modulate the expression of an endogenous mogroside synthesis pathway gene, can have an increase in sweetness of at least 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more, compared to an extract from a control plant without the modification to modulate the expression of an endogenous mogroside synthesis pathway gene. For example, a sweetener from a plant, wherein the genome of a cell or part of the plant is genetically-modified to modulate the expression of an endogenous mogroside synthesis pathway gene, can have an increase in sweetness of about 20-1000%, about 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more, or about 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more, compared to an extract from a control plant without the modification to modulate the expression of an endogenous mogroside synthesis pathway gene.

Additionally, or alternatively, a sweetener from a plant disclosed herein can have a glycemic index (GI) of about 10 or less (e.g., about 10, 9, 8, 7, 6, 5, 4, 3, 2, 1, or less), such as a GI of about 5 or less (e.g., about 5, 4, 3, 2, 1, or less). In particular, a sweetener from a plant disclosed herein can have a GI of 0. In certain embodiments, GI of a sweetener from a plant described herein is measured by relative rise in blood glucose level two hours after consuming the sweetener. For example, GI of a sweetener from a plant described herein can be measured based on the incremental area under two-hour blood glucose response curve (AUC) following a 12-hour fast and ingestion of the sweetener with a certain quantity of available carbohydrate (e.g., about 50 g). The AUC of the sweetener can be divided by the AUC of a standard (e.g., glucose or white bread) and multiplied by 100 to obtain the GI value of the sweetener. The average GI value can be calculated from data collected in 10 human subjects.

The present disclosure also describes plant parts (e.g., leaves, stems, roots, bark, flowers, fruits, seeds, pulp, juice, nectar, embryos, pollen, ovules, branches, kernels, ears, cobs, husks, stalks, root tips, anthers, etc.) from plants that are disclosed herein above, including plants, wherein the genome of a cell or part of the plant is genetically-modified to modulate the expression of one or more endogenous mogroside synthesis pathway genes. Plants parts encompassed herein can be proliferative plant parts, including but not limited to seeds, or non-proliferative plants parts including juice, pulp, fruit, flowers, nectar, leaves, stems, branches, bark, kernels, ears, cobs, husks, stalks, roots, root tips, etc.

In some embodiments, a plant part of a plant disclosed herein (e.g., a plant wherein the genome of a cell or part of the plant is genetically-modified by the methods disclosed herein to modulate the expression of an endogenous mogroside synthesis pathway gene) has increased amount of at least one mogroside. In certain embodiments, a plant part of a plant disclosed herein has an increase in the level of at least one mogroside of about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more mogroside compared to the level of the mogroside in plant part of a control plant.

In some embodiments, a plant part disclosed herein has a high sweetness index. In certain instances, the sweetness index of a plant part disclosed herein is measured relative to cane sugar. In particular, sweetness index relative to cane sugar can be measured by comparing threshold value of the plant part with the threshold value of cane sugar. Threshold value of the plant part or cane sugar may indicate the concentration at which the presence of the plant part or cane sugar in water can be detected by half of a panel of trained tasters. For example, researchers may present each participant of a panel with samples of water to which varying degrees of the plant part or cane sugar has been added. First, the tasters may be given plain water, and then, samples of water with increasing concentrations of the plant part or cane sugar until the presence of the plant part or cane sugar is detected (e.g., tasted) in the water samples by the tasters. Concentration of the plant part or cane sugar at which half of the panel of tasters can detect a change in the water sample (e.g., a change in taste of the water sample) may be considered as the threshold value of the plant part or cane sugar. Threshold value of the plant part can then be compared to cane sugar to determine the sweetness index of the plant part relative to cane sugar.

In some embodiments, a plant part of a plant disclosed herein has a sweetness index of at least 50×, 100×, 150×, 200×, 250×, 300×, 350×, 400×, 450×, 500×, 550×, 600×, 650×, 700×, 750×, 800×, 850×, 900×, 950×, 1000×, compared to cane sugar. For example, a plant part (e.g., juice, pulp, seed, fruit, flowers, nectar, embryos, pollen, ovules, leaves, stems, branches, bark, kernels, ears, cobs, husks, stalks, roots, root tips, anthers, etc.) of a plant disclosed herein can have a sweetness index of about 50-1000×, about 50-300×, 200-450×, 350-600×, 500-750×, 750-1000×, or about 50×, 100×, 150×, 200×, 250×, 300×, 350×, 400×, 450×, 500×, 550×, 600×, 650×, 700×, 750×, 800×, 850×, 900×, 950×, 1000×, or more compared to cane sugar.

Alternatively, a plant part from a plant, wherein the genome of a cell or part of the plant is genetically-modified to modulate the expression of an endogenous mogroside synthesis pathway gene, can have an increase in sweetness of at least 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more, compared to a plant part from a control plant without the modification to modulate the expression of an endogenous mogroside synthesis pathway gene. For example, a plant part (e.g., juice, pulp, seed, fruit, flowers, nectar, embryos, pollen, ovules, leaves, stems, branches, bark, kernels, ears, cobs, husks, stalks, roots, root tips, anthers, etc.) from a plant, wherein the genome of a cell or part of the plant is genetically-modified to modulate the expression of an endogenous mogroside synthesis pathway gene, can have an increase in sweetness of about 20-1000%, or more (e.g., about 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more (e.g., about 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more)) compared to a plant part from a control plant without the modification to modulate the expression of an endogenous mogroside synthesis pathway gene.

A plant part of a plant disclosed herein can have a GI of about 10 or less (e.g., about 10, 9, 8, 7, 6, 5, 4, 3, 2, 1, or less), such as a GI of about 5 or less (e.g., about 5, 4, 3, 2, 1, or less). In particular, a plant part of a plant disclosed herein can have a GI of 0. In certain embodiments, GI of a plant part described herein is measured by relative rise in blood glucose level two hours after consuming the plant part. For example, GI of a plant part from a plant described herein can be measured based on the incremental area under two-hour blood glucose response curve (AUC) following a 12-hour fast and ingestion of the plant part with a certain quantity of available carbohydrate (e.g., about 50 g). The AUC of the plant part can be divided by the AUC of a standard (e.g., glucose or white bread) and multiplied by 100 to obtain the GI value of the plant part. The average GI value can be calculated from data collected in 10 human subjects.

In some embodiments, the plant part is a seed (e.g., seed from fruit of the plant). For example, the present disclosure may describe a seed from a plant that is disclosed hereinabove, such as plants wherein the genome of a cell or part of the plant is genetically-modified to modulate the expression of one or more endogenous mogroside synthesis pathway genes. In some embodiments, a seed from a plant disclosed herein has modulated expression of one or more endogenous genes of the mogroside synthesis pathway. In certain embodiments, the expression of one or more endogenous genes of the mogroside synthesis pathway is modulated by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more, in a seed from a plant disclosed herein compared to a seed from a control plant. In particular embodiments, the expression of one or more endogenous genes of the mogroside synthesis pathway is increased by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more, in a seed from a plant disclosed herein compared to a seed from a control plant.

In some embodiments, the plant part is juice (e.g., juice from fruit of the plant, juice from leaf of the plant, etc.). For example, the present disclosure may describe juice from a plant that is disclosed hereinabove, including plants wherein the genome of a cell or part of the plant is genetically-modified to modulate expression of one or more endogenous mogroside synthesis pathway genes. In some embodiments, juice from a plant disclosed herein has increased amount of at least one mogroside. In certain embodiments, juice from a plant disclosed herein has an increase in the level of at least one mogroside of about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more, mogroside compared to the level of the mogroside in juice of a control plant. In some embodiments, juice from a plant disclosed herein has a high sweetness index. In certain instances, the sweetness index of a juice from a plant disclosed herein is measured relative to cane sugar. In particular, sweetness index relative to cane sugar can be measured by comparing threshold value of the juice with the threshold value of cane sugar as disclosed elsewhere herein.

In some embodiments, juice from a plant disclosed herein (e.g., a plant wherein the genome of a cell or part of the plant is genetically-modified to modulate the expression of an endogenous mogroside synthesis pathway gene by the methods disclosed herein) has a sweetness index of at least 50×, 100×, 150×, 200×, 250×, 300×, 350×, 400×, 450×, 500×, 550×, 600×, 650×, 700×, 750×, 800×, 850×, 900×, 950×, 1000×, compared to cane sugar. For example, juice from a plant disclosed herein can have a sweetness index of about 50-1000×, about 50-300×, 200-450×, 350-600×, 500-750×, 750-1000×, or about 50×, 100×, 150×, 200×, 250×, 300×, 350×, 400×, 450×, 500×, 550×, 600×, 650×, 700×, 750×, 800×, 850×, 900×, 950×, 1000×, or more compared to cane sugar.

Alternatively, juice from a plant (e.g., juice from fruit of a plant, juice from leaf of a plant, etc.), wherein the genome of a cell or part of the plant is genetically-modified to modulate the expression of an endogenous mogroside synthesis pathway gene, can have an increase in sweetness of at least 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more, compared to juice from a control plant without the modification to modulate the expression of an endogenous mogroside synthesis pathway gene. For example, juice from a plant wherein the genome of a cell or part of the plant is genetically-modified to modulate the expression of an endogenous mogroside synthesis pathway gene can have an increase in sweetness of about 20-1000%, or more, or about 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more, or about 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more, compared to juice from a control plant without the modification to modulate the expression of an endogenous mogroside synthesis pathway gene.

Additionally, or alternatively, juice from a plant disclosed herein can have a GI of about 10 or less (e.g., about 10, 9, 8, 7, 6, 5, 4, 3, 2, 1, or less), such as a GI of about 5 or less (e.g., about 5, 4, 3, 2, 1, or less). In particular, juice from a plant disclosed herein can have a GI of 0. In certain embodiments, GI of a juice from a plant described herein is measured by relative rise in blood glucose level two hours after consuming the juice. For example, GI of a juice from a plant described herein can be measured based on the incremental area under two-hour blood glucose response curve (AUC) following a 12-hour fast and ingestion of the juice with a certain quantity of available carbohydrate (e.g., about 50 g). The AUC of the juice can be divided by the AUC of a standard (e.g., glucose or white bread) and multiplied by 100 to obtain the GI value of the juice. The average GI value can be calculated from data collected in 10 human subjects.

In some embodiments, the plant part is pulp (e.g., pulp from fruit of the plant, pulp from stem of the plant, etc.). For example, the present disclosure may describe pulp from a plant that is disclosed hereinabove, such as plants wherein the genome of a cell or part of the plant is genetically-modified to modulate expression of one or more endogenous mogroside synthesis pathway genes. In some embodiments, pulp from a plant disclosed herein has increased amount of at least one mogroside. In certain embodiments, pulp from a plant disclosed herein has an increase in the level of at least one mogroside of about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more mogroside compared to the level of the mogroside in pulp of a control plant.

In some embodiments, pulp from a plant disclosed herein has a high sweetness index. In certain instances, the sweetness index of a pulp from a plant disclosed herein is measured relative to cane sugar. In particular, sweetness index relative to cane sugar can be measured by comparing threshold value of the pulp with the threshold value of cane sugar. Threshold value of the pulp or cane sugar may indicate the concentration at which the presence of the pulp or cane sugar in water can be detected by half of a panel of trained tasters. For example, researchers may present each participant of a panel with samples of water to which varying degrees of the pulp or cane sugar has been added. First, the tasters may be given plain water, and then, samples of water with increasing concentrations of the pulp or cane sugar until the presence of the pulp or cane sugar is detected (e.g., tasted) in the water samples by the tasters. Concentration of the pulp or cane sugar at which half of the panel of tasters can detect a change in the water sample (e.g., a change in taste of the water sample) may be considered as the threshold value of the pulp or cane sugar. Threshold value of the pulp can then be compared to cane sugar to determine the sweetness index of the pulp relative to cane sugar.

In some embodiments, pulp from a plant (e.g., pulp from fruit of a plant, pulp from stem of a plant, etc.) disclosed herein has a sweetness index of at least 50×, 100×, 150×, 200×, 250×, 300×, 350×, 400×, 450×, 500×, 550×, 600×, 650×, 700×, 750×, 800×, 850×, 900×, 950×, 1000×, compared to cane sugar. For example, pulp from a plant disclosed herein can have a sweetness index of about 50-1000×, about 50-300×, 200-450×, 350-600×, 500-750×, 750-1000×, or about 50×, 100×, 150×, 200×, 250×, 300×, 350×, 400×, 450×, 500×, 550×, 600×, 650×, 700×, 750×, 800×, 850×, 900×, 950×, 1000×, or more compared to cane sugar.

Alternatively, pulp from a plant, wherein the genome of a cell or part of the plant is genetically-modified to modulate the expression of an endogenous mogroside synthesis pathway gene, can have an increase in sweetness of at least 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more, compared to pulp from a control plant without the modification to modulate the expression of an endogenous mogroside synthesis pathway gene. For example, pulp from a plant (e.g., pulp from fruit of a plant, pulp from stem of a plant, etc.), wherein the genome of a cell or part of the plant is genetically-modified to modulate the expression of an endogenous mogroside synthesis pathway gene, can have an increase in sweetness of about 20-1000%, about 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or about 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more, compared to pulp from a control plant without the modification to modulate the expression of an endogenous mogroside synthesis pathway gene.

Additionally, or alternatively, pulp from a plant disclosed herein can have a GI of about 10 or less (e.g., about 10, 9, 8, 7, 6, 5, 4, 3, 2, 1, or less), such as a GI of about 5 or less (e.g., about 5, 4, 3, 2, 1, or less). In particular, pulp from a plant disclosed herein can have a GI of 0. In certain embodiments, GI of pulp from a plant described herein is measured by relative rise in blood glucose level two hours after consuming the pulp. For example, GI of pulp from a plant described herein can be measured based on the incremental area under two-hour blood glucose response curve (AUC) following a 12-hour fast and ingestion of the pulp with a certain quantity of available carbohydrate (e.g., about 50 g). The AUC of the pulp can be divided by the AUC of a standard (e.g., glucose or white bread) and multiplied by 100 to obtain the GI value of the pulp. The average GI value can be calculated from data collected in 10 human subjects.

In some embodiments, pulp from a plant disclosed herein has modulated expression of one or more endogenous genes of the mogroside synthesis pathway. In certain embodiments, the expression of one or more endogenous genes of the mogroside synthesis pathway is modulated by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more in pulp from a plant disclosed herein compared to pulp from a control plant. In particular embodiments, the expression of one or more endogenous genes of the mogroside synthesis pathway is increased by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more in pulp from a plant disclosed herein compared to pulp from a control plant (e.g., a plant without the modification to modulate the expression of an endogenous mogroside synthesis pathway gene).

The present disclosure also describes biomass from plants that are disclosed hereinabove, such as plants wherein the genome of a cell or part of the plant is genetically-modified to modulate expression of one or more endogenous mogroside synthesis pathway genes. In certain embodiments, a plant biomass described herein may be dried biomass (e.g., biomass dried by methods including, but not limited to, freeze drying, dessication, and/or spray drying). In particular embodiments, a dried biomass may be crushed and/or powdered to form crushed and/or powdered biomass. In some embodiments, biomass from a plant (e.g., dried biomass, such as crushed and/or powdered biomass of a plant) disclosed herein has increased amount of at least one mogroside. In certain embodiments, biomass from a plant disclosed herein has an increase in the level of at least one mogroside of about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more mogroside compared to the level of the mogroside in biomass of a control plant (e.g., a plant without the modification to modulate the expression of an endogenous mogroside synthesis pathway gene).

In some embodiments, biomass from a plant disclosed herein has a high sweetness index, as measured by the methods disclosed elsewhere herein. In certain instances, the sweetness index of a biomass from a plant disclosed herein is measured relative to cane sugar. In particular, sweetness index relative to cane sugar can be measured by comparing threshold value of the biomass with the threshold value of cane sugar. Threshold value of the biomass or cane sugar may indicate the concentration at which the presence of the biomass or cane sugar in water can be detected by half of a panel of trained tasters. For example, researchers may present each participant of a panel with samples of water to which varying degrees of the biomass or cane sugar has been added. First, the tasters may be given plain water, and then, samples of water with increasing concentrations of the biomass or cane sugar until the presence of the biomass or cane sugar is detected (e.g., tasted) in the water samples by the tasters. Concentration of the biomass or cane sugar at which half of the panel of tasters can detect a change in the water sample (e.g., a change in taste of the water sample) may be considered as the threshold value of the biomass or cane sugar. Threshold value of the biomass can then be compared to cane sugar to determine the sweetness index of the biomass relative to cane sugar.

In some embodiments, biomass from a plant disclosed herein has a sweetness index of at least 50×, 100×, 150×, 200×, 250×, 300×, 350×, 400×, 450×, 500×, 550×, 600×, 650×, 700×, 750×, 800×, 850×, 900×, 950×, 1000×, compared to cane sugar. For example, biomass from a plant disclosed herein can have a sweetness index of about 50-1000×, about 50-300×, 200-450×, 350-600×, 500-750×, 750-1000×, or about 50×, 100×, 150×, 200×, 250×, 300×, 350×, 400×, 450×, 500×, 550×, 600×, 650×, 700×, 750×, 800×, 850×, 900×, 950×, 1000×, or more compared to cane sugar.

Alternatively, biomass from a plant, wherein the genome of a cell or part of the plant is genetically-modified to modulate the expression of an endogenous mogroside synthesis pathway gene, can have an increase in sweetness of at least 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more, compared to biomass from a control plant without the modification to modulate the expression of an endogenous mogroside synthesis pathway gene. For example, biomass from a plant (e.g., dried biomass, such as crushed and/or powdered biomass of a plant), wherein the genome of a cell or part of the plant is genetically-modified to modulate the expression of an endogenous mogroside synthesis pathway gene, can have an increase in sweetness of about 20-1000%, or more (e.g., about 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more (e.g., about 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more)) compared to biomass from a control plant without the genetic modification to modulate the expression of an endogenous mogroside synthesis pathway gene.

Additionally, or alternatively, biomass from a plant disclosed herein can have a GI of about 10 or less (e.g., about 10, 9, 8, 7, 6, 5, 4, 3, 2, 1, or less), such as a GI of about 5 or less (e.g., about 5, 4, 3, 2, 1, or less). In particular, biomass from a plant disclosed herein can have a GI of 0. In certain embodiments, GI of biomass from a plant described herein is measured by relative rise in blood glucose level two hours after consuming the biomass. For example, GI of biomass from a plant described herein can be measured based on the incremental area under two-hour blood glucose response curve (AUC) following a 12-hour fast and ingestion of the biomass with a certain quantity of available carbohydrate (e.g., about 50 g). The AUC of the biomass can be divided by the AUC of a standard (e.g., glucose or white bread) and multiplied by 100 to obtain the GI value of the biomass. The average GI value can be calculated from data collected in 10 human subjects.

In some embodiments, biomass from a plant disclosed herein has modulated expression of one or more endogenous genes of the mogroside synthesis pathway. In certain embodiments, the expression of one or more endogenous genes of the mogroside synthesis pathway is modulated by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more, in biomass from a plant disclosed herein compared to biomass from a control plant. In particular embodiments, the expression of one or more endogenous genes of the mogroside synthesis pathway is increased by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more in biomass from a plant disclosed herein compared to biomass from a control plant.

The present disclosure also describes concentrates from plants (e.g., a Cucurbitaceae family plant, such as a watermelon plant or a cucumber plant) that are disclosed hereinabove, such as plants wherein the genome of a cell or part of the plant is genetically-modified to modulate expression of one or more endogenous mogroside synthesis pathway genes. In some embodiments, the plant concentrate disclosed herein may be a whole plant concentrate that is obtained by subjecting a whole plant to one or more mechanical processes known in the art, including but not limited to compressing, dry sifting, crushing (e.g., drying followed by crushing), grinding (e.g., drying followed by grinding), and/or blending (e.g., drying followed by blending). Additionally, or alternatively, the plant concentrate disclosed herein may be a plant part concentrate that is obtained by subjecting one or more plant parts to one or more mechanical processes known in the art, including but not limited to compressing, dry sifting, crushing, grinding, and/or blending. In certain embodiments, the plant concentrate (e.g., whole plant concentrate or plant part concentrate) from the plant is one or more mogroside. In other embodiments, the plant concentrate from the plant comprises one or more mogrosides. In some embodiments, a plant concentrate from a plant disclosed herein has increased amount of at least one mogroside. In certain embodiments, a plant from a plant disclosed herein has an increase in the level of at least one mogroside of about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more mogroside compared to the level of the mogroside in a plant concentrate of a control plant.

In some embodiments, a plant concentrate (e.g., whole plant concentrate or plant part concentrate) from a plant disclosed herein has a high sweetness index. In certain instances, the sweetness index of a plant concentrate from a plant disclosed herein is measured relative to cane sugar. In particular, sweetness index relative to cane sugar can be measured by comparing threshold value of the plant concentrate with the threshold value of cane sugar. Threshold value of the plant concentrate or cane sugar may indicate the concentration at which the presence of the plant concentrate or cane sugar in water can be detected by half of a panel of trained tasters. For example, researchers may present each participant of a panel with samples of water to which varying degrees of the plant concentrate or cane sugar has been added. First, the tasters may be given plain water, and then, samples of water with increasing concentrations of the plant concentrate or cane sugar until the presence of the plant concentrate or cane sugar is detected (e.g., tasted) in the water samples by the tasters. Concentration of the plant concentrate or cane sugar at which half of the panel of tasters can detect a change in the water sample (e.g., a change in taste of the water sample) may be considered as the threshold value of the plant concentrate or cane sugar. Threshold value of the plant concentrate can then be compared to cane sugar to determine the sweetness index of the plant concentrate relative to cane sugar.

In some embodiments, a plant concentrate (e.g., whole plant concentrate or plant part concentrate) from a plant disclosed herein (e.g., a plant wherein the genome of a cell or part of the plant is genetically-modified to modulate the expression of an endogenous mogroside synthesis pathway gene by the methods disclosed herein) has a sweetness index of at least 50×, 100×, 150×, 200×, 250×, 300×, 350×, 400×, 450×, 500×, 550×, 600×, 650×, 700×, 750×, 800×, 850×, 900×, 950×, 1000×, or more, compared to cane sugar. For example, a concentrate from a plant disclosed herein can have a sweetness index of about 50-1000×, about 50-300×, 200-450×, 350-600×, 500-750×, 750-1000×, or about 50×, 100×, 150×, 200×, 250×, 300×, 350×, 400×, 450×, 500×, 550×, 600×, 650×, 700×, 750×, 800×, 850×, 900×, 950×, 1000×, or more, compared to cane sugar.

Plant concentrate from a plant, wherein the genome of a cell or part of the plant is genetically-modified to modulate the expression of an endogenous mogroside synthesis pathway gene, can have an increase in sweetness of at least 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more, compared to plant concentrate from a control plant without the modification to modulate the expression of an endogenous mogroside synthesis pathway gene. For example, plant concentrate from a plant wherein the genome of a cell or part of the plant is genetically-modified to modulate the expression of an endogenous mogroside synthesis pathway gene can have an increase in sweetness of about 20-1000%, or more (e.g., about 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more (e.g., about 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more)) compared to plant concentrate from a control plant without the modification to modulate the expression of an endogenous mogroside synthesis pathway gene.

Additionally, or alternatively, a plant concentrate from a plant disclosed herein can have a GI of about 10 or less (e.g., about 10, 9, 8, 7, 6, 5, 4, 3, 2, 1, or less), such as a GI of about 5 or less (e.g., about 5, 4, 3, 2, 1, or less). In particular, a plant concentrate from a plant disclosed herein can have a GI of 0. In certain embodiments, GI of a plant concentrate from a plant described herein is measured by relative rise in blood glucose level two hours after consuming the plant concentrate. For example, GI of a plant concentrate from a plant described herein can be measured based on the incremental area under two-hour blood glucose response curve (AUC) following a 12-hour fast and ingestion of the plant concentrate with a certain quantity of available carbohydrate (e.g., about 50 g). The AUC of the concentrate can be divided by the AUC of a standard (e.g., glucose or white bread) and multiplied by 100 to obtain the GI value of the plant concentrate. The average GI value can be calculated from data collected in 10 human subjects.

In some embodiments, a plant concentrate (e.g., whole plant concentrate or plant part concentrate) from a plant disclosed herein has modulated (e.g., increased or decreased) expression of one or more endogenous genes of the mogroside synthesis pathway. In certain embodiments, the expression of one or more endogenous genes of the mogroside synthesis pathway is modulated by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more, in a plant concentrate from a plant disclosed herein compared to a plant concentrate from a control plant. In particular embodiments, the expression of one or more endogenous genes of the mogroside synthesis pathway is increased by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more in a plant concentrate from a plant disclosed herein compared to a plant concentrate from a control plant (e.g., a plant without the modification to modulate the expression of an endogenous mogroside synthesis pathway gene).

The present disclosure also describes powders from plants that are disclosed hereinabove, such as plants wherein the genome of a cell or part of the plant is genetically-modified to modulate expression of one or more endogenous mogroside synthesis pathway genes. In some embodiments, the plant powder disclosed herein may be a formulated powder. In particular, the formulated plant powder disclosed herein may be a formulated plant part powder that is obtained by drying (e.g., drying by methods including, but not limited to, freeze drying, dessication, and/or spray drying), crushing, grinding, and/or blending a plant part (e.g., one or more of the plant parts described hereinabove). In certain embodiments, the plant powder (e.g., formulated powder, such as formulated plant part powder) from the plant is one or more mogroside. In other embodiments, the plant powder from the plant comprises one or more mogrosides. In particular embodiments, a plant powder from a plant disclosed herein has increased amount of at least one mogroside. For example, a plant powder (e.g., formulated powder, such as formulated plant part powder) from a plant disclosed herein may have an increase in the level of at least one mogroside of about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more, mogroside compared to the level of the mogroside in a plant powder from a control plant.

In some embodiments, a plant powder from a plant disclosed herein has a high sweetness index. In certain instances, the sweetness index of a plant powder from a plant disclosed herein is measured relative to cane sugar. In particular, sweetness index relative to cane sugar can be measured by comparing threshold value of the plant powder with the threshold value of cane sugar. Threshold value of the plant powder or cane sugar may indicate the concentration at which the presence of the plant powder or cane sugar in water can be detected by half of a panel of trained tasters. For example, researchers may present each participant of a panel with samples of water to which varying degrees of the plant powder or cane sugar has been added. First, the tasters may be given plain water, and then, samples of water with increasing concentrations of the plant powder) or cane sugar until the presence of the plant powder or cane sugar is detected (e.g., tasted) in the water samples by the tasters. Concentration of the plant powder or cane sugar at which half of the panel of tasters can detect a change in the water sample may be considered as the threshold value of the plant powder or cane sugar. Threshold value of the plant powder can then be compared to cane sugar to determine the sweetness index of the plant powder relative to cane sugar.

In some embodiments, a plant powder from a plant disclosed herein (e.g., a plant wherein the genome of a cell or part of the plant is genetically-modified to modulate the expression of an endogenous mogroside synthesis pathway gene by the methods disclosed herein) has a sweetness index of at least 50×, 100×, 150×, 200×, 250×, 300×, 350×, 400×, 450×, 500×, 550×, 600×, 650×, 700×, 750×, 800×, 850×, 900×, 950×, 1000×, or more, compared to cane sugar. For example, a plant powder from a plant disclosed herein can have a sweetness index of about 50-1000×, about 50-300×, 200-450×, 350-600×, 500-750×, 750-1000×, or about 50×, 100×, 150×, 200×, 250×, 300×, 350×, 400×, 450×, 500×, 550×, 600×, 650×, 700×, 750×, 800×, 850×, 900×, 950×, 1000×, or more, compared to cane sugar.

Alternatively, a plant powder from a plant, wherein the genome of a cell or part of the plant is genetically-modified to modulate the expression of an endogenous mogroside synthesis pathway gene, can have an increase in sweetness of at least 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more, compared to plant powder from a control plant without the modification to modulate the expression of an endogenous mogroside synthesis pathway gene. For example, a plant powder from a plant, wherein the genome of a cell or part of the plant is genetically-modified to modulate the expression of an endogenous mogroside synthesis pathway gene, can have an increase in sweetness of about 20-1000%, or more (e.g., about 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more (e.g., about 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more)) compared to plant powder from a control plant without the modification to modulate the expression of an endogenous mogroside synthesis pathway gene.

A plant powder from a plant disclosed herein (e.g., a plant wherein the genome of a cell or part of the plant is genetically-modified to modulate the expression of an endogenous mogroside synthesis pathway gene by the methods disclosed herein) can have a GI of about 10 or less (e.g., about 10, 9, 8, 7, 6, 5, 4, 3, 2, 1, or less), such as a GI of about 5 or less (e.g., about 5, 4, 3, 2, 1, or less). In particular, a plant powder from a plant disclosed herein can have a GI of 0. In certain embodiments, GI of a plant powder from a plant described herein is measured by relative rise in blood glucose level two hours after consuming the powder. For example, GI of a plant powder from a plant described herein can be measured based on the incremental area under two-hour blood glucose response curve (AUC) following a 12-hour fast and ingestion of the plant powder with a certain quantity of available carbohydrate (e.g., about 50 g). The AUC of the plant powder can be divided by the AUC of a standard (e.g., glucose or white bread) and multiplied by 100 to obtain the GI value of the plant powder. The average GI value can be calculated from data collected in 10 human subjects.

In some embodiments, a plant powder from a plant disclosed herein has modulated expression of one or more endogenous genes of the mogroside synthesis pathway. In certain embodiments, the expression of one or more endogenous genes of the mogroside synthesis pathway is modulated by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more, in a plant powder from a plant disclosed herein compared to a plant powder from a control plant. In particular embodiments, the expression of one or more endogenous genes of the mogroside synthesis pathway is increased by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more, in a plant powder from a plant disclosed herein compared to a plant powder from a control plant.

### 2.3 Modulating the Expression of Endogenous Mogroside Synthesis Pathway Genes

Described herein are methods for genetically-modifying the genome of a plant cell or plant part to modulate the expression of one or more endogenous genes of the mogroside synthesis pathway in order to increase the level of at least one mogroside in the cell or plant part. In some embodiments of the present disclosure, the expression of one or more endogenous genes of the mogroside synthesis pathway is modulated (e.g., increased or decreased) by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more compared to a control plant (e.g., a plant without the modification to modulate the expression of an endogenous mogroside synthesis pathway gene). In particular embodiments, the expression of one or more endogenous genes of the mogroside synthesis pathway is increased by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more compared to a control plant.

Mogrosides are derived from the cucurbitane skeleton of triterpenoids, one of hundreds of possible cyclic triterpenoid backbones (Xu et al., Phytochemistry 65:261-291 (2004); Thimmappa et al., Annu Rev Plant Biol 65:225-257 (2014)), which is ubiquitous throughout the Cucurbitaceae family. The intensely bitter cucurbitacins are derivatives of the same skeleton, but differ from the intensely sweet mogrosides primarily in the oxygenated decorations on the cucurbitane backbone. Mogrosides are unique among the cucurbitane triterpenoids in view of their four regio-specific oxygenations, at C3, C11, C24, and C25, forming the tetra-hydroxylated cucurbitane, mogrol (Itkin et al., Proc Natl Acad Sci 113: E7619-E7628 (2016)).

The mogroside synthesis pathway can be divided into upstream and downstream pathways (Shi et al., Molecules 24:4076 (2019)). Six enzyme families play pivotal roles in the upstream pathway; acetyl-CoA is transformed to mogrol via catalysis by 3-hydroxy-3-methyl glutaryl coenzyme A reductase (TIMGR), squalene synthase (SQS), squalene epoxidase (SQE), cucurbitadienol synthase (CDS), epoxide hydrolase (EPH), and cytochrome P450 (CYP450) (Tang et al., BMC Genom 12:343 (2011)). Squalene is the precursor of 2,3-Oxidosqualene, and 2,3-Oxidosqualene can transform into cucurbitadienol under CDS catalysis. Cucurbitadienol, as a crucial triterpenoid skeleton, can be converted to a series of mogrosides. Cycloartenol, the starting point of synthesis of almost all plant steroids, competes for substrate 2,3-Oxidosqualene with the cucurbitadienol biosynthesis in monk fruit. In the downstream portion of the mogroside biosynthetic pathway, mogrol is transformed into different grades of mogrosides by UDP-glycosyltransferases (UGTs). Overall, twenty-four structural genes are considered to be involved in the in mogroside synthesis pathway (Xia et al., Gigascience 7:1-9 (2018); U.S. Patent Application Publication No. US 2017/0283844 A1). A summary schematic of the mogroside synthesis pathway is provided in FIG. 1.

(A) Mogroside Synthesis Pathway Genes

In some embodiments of the present disclosure, the one or more endogenous mogroside synthesis pathway genes are selected from: a gene encoding cucurbitadienol synthase (CDS; also known as (S)-2,3-epoxysqualene mutase (cyclizing, cucurbitadienol-forming); systematic name: (3S)-2,3-epoxy-2,3-dihydrosqualene mutase (cyclizing, cucurbitadienol-forming)) (SEQ ID NO: 15) or a gene encoding a polypeptide sequence having at least 75% (e.g., at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity thereto; a gene encoding cytochrome P450 enzyme 87D18 (CYP87D18; also known as cucurbitadienol 11-hydroxylase) (SEQ ID NO: 16) or a gene encoding a polypeptide sequence having at least 75% (e.g., at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity thereto; a gene encoding epoxide hydrolase 3 (EPH3; also known as ABHD9, EH3, or EPHX3) (SEQ ID NO: 17) or a gene encoding a polypeptide sequence having at least 75% (e.g., at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity thereto; a gene encoding squalene epoxidase 1 (SQE1; also known as squalene monooxygenase) (SEQ ID NO: 18) or a gene encoding a polypeptide sequence having at least 75% (e.g., at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity thereto; a gene encoding UDP glycosyltransferase 720 (UGT720) (SEQ ID NO: 19) or a gene encoding a polypeptide sequence having at least 75% (e.g., at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity thereto; and/or a gene encoding UDP glycosyltransferase 94 (UGT94) (SEQ ID NO: 20) or a gene encoding a polypeptide sequence having at least 75% (e.g., at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity thereto. In specific embodiments, the one or more mogroside synthesis pathway genes are endogenous mogroside synthesis pathway genes.

The one or more endogenous mogroside synthesis pathway genes can be a homolog of a gene encoding CDS, CYP87D18, EPH3, SQE1, UGT720, and/or UGT94. For example, the one or more endogenous mogroside synthesis pathway genes can be selected from: one or more homolog of a gene encoding CDS; one or more homolog of a gene encoding CYP87D18; one or more homolog of a gene encoding EPH3; one or more homolog of a gene encoding SQE1; one or more homolog of a gene encoding UGT720; and/or one or more homolog of a gene encoding UGT94. In particular embodiments, the one or more mogroside synthesis pathway genes are selected from genes encoding polypeptide sequences having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth in any one of SEQ ID NOs: 1-14 and 21.

In some embodiments of the present disclosure, the expression of a gene encoding CDS (SEQ ID NO: 15) or a gene encoding a polypeptide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity thereto is modulated (e.g., increased or decreased) by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more.

CYP87D18 acts as a multifunctional cucurbitadienol oxidase in the mogrosides synthesis pathway (Zhang et al., Plant Cell Physiol 57:1000-1007 (2016)). In some embodiments, the expression of a gene encoding CYP87D18 (SEQ ID NO: 16) or a gene encoding a polypeptide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity thereto can be modulated (e.g., increased or decreased) by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more. For example, variants of CYP87D18 can differ from the amino acid sequence of SEQ ID NO: 16 by a single amino acid position. In specific embodiments, the variant or homolog of CYP87D18 is CYP 102801 set forth in SEQ ID NO: 21.

The expression of a gene encoding EPH3 (SEQ ID NO: 17) or a gene encoding a polypeptide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity thereto can be modulated (e.g., increased or decreased) by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more.

The expression of a gene encoding SQE1 (SEQ ID NO: 18) or a gene encoding a polypeptide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity thereto can be modulated (e.g., increased or decreased) by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more.

The expression of a gene encoding UGT720 (SEQ ID NO: 19) or a gene encoding a polypeptide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity thereto can be modulated (e.g., increased or decreased) by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more.

The expression of a gene encoding UGT94 (SEQ ID NO: 20) or a gene encoding a polypeptide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity thereto can be modulated (e.g., increased or decreased) by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more.

The expression of a gene encoding a polypeptide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth in SEQ ID NO: 1 can be modulated (e.g., increased or decreased) by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more. The expression of a gene encoding a polypeptide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth in SEQ ID NO: 2 can be modulated (e.g., increased or decreased) by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more.

The expression of a gene encoding a polypeptide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth in SEQ ID NO: 3 can be modulated (e.g., increased or decreased) by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more. The expression of a gene encoding a polypeptide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth in SEQ ID NO: 4 can be modulated (e.g., increased or decreased) by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more.

The expression of a gene encoding a polypeptide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth in SEQ ID NO: 5 can be modulated (e.g., increased or decreased) by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more.

The expression of a gene encoding a polypeptide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth in SEQ ID NO: 6 can be modulated (e.g., increased or decreased) by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more.

The expression of a gene encoding a polypeptide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth in SEQ ID NO: 7 can be modulated (e.g., increased or decreased) by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more. The expression of a gene encoding a polypeptide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth in SEQ ID NO: 8 can be modulated (e.g., increased or decreased) by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more.

The expression of a gene encoding a polypeptide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth in SEQ ID NO: 9 can be modulated (e.g., increased or decreased) by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more.

The expression of a gene encoding a polypeptide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth in SEQ ID NO: 10 can be modulated (e.g., increased or decreased) by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more.

The expression of a gene encoding a polypeptide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth in SEQ ID NO: 11 can be modulated (e.g., increased or decreased) by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more.

The expression of a gene encoding a polypeptide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth in SEQ ID NO: 12 can be modulated (e.g., increased or decreased) by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more.

The expression of a gene encoding a polypeptide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth in SEQ ID NO: 13 can be modulated (e.g., increased or decreased) by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more.

The expression of a gene encoding a polypeptide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth in SEQ ID NO: 14 can be modulated (e.g., increased or decreased) by about 1-10%, 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, or more.

(B) Methods for Modulating Mogroside Synthesis Pathway Genes

Also disclosed herein are methods for genetically-modifying the genome of a plant cell or plant part to modulate (e.g., increase or decrease) the expression of one or more endogenous mogroside synthesis pathway genes in order to increase the level of mogrosides in the cell or plant part. In some embodiments of the present disclosure, the genome of a plant cell or plant part is genetically-modified by one or more of the following methods to modulate the expression of one or more endogenous mogroside synthesis pathway genes.

(i) Modification of Promoter Sequence

In some embodiments of the present disclosure, genetically-modifying the genome of a plant cell or plant part to modulate the expression of one or more endogenous mogroside synthesis pathway genes encompasses modification (e.g., activation or inactivation) of the promoter sequence of the respective gene. For example, to modulate the expression of one or more endogenous mogroside synthesis pathway genes, the genome of a plant cell or plant part can be genetically-modified by activation of the promoter sequence of the respective gene. In particular embodiments, the promoter sequence of a mogroside synthesis pathway gene is not active or exhibits decreased activity. By increasing the activity of the promoter sequence, the expression of the gene(s) operably linked to the promoter can be modulated in order to increase the level of mogrosides.

In certain embodiments, the promoter sequence of the gene (e.g., endogenous gene) is modified by insertion of one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more) nucleotides. For example, the activity of the promoter sequence of the gene (e.g., endogenous gene) can be enhanced, improved, or increased by insertion of one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more) nucleotides. Additionally or alternatively, the promoter sequence of the gene (e.g., endogenous gene) can be modified by deletion of one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more) nucleotides. For example, the activity of the promoter sequence of the gene (e.g., endogenous gene) can be enhanced, improved, or increased by deletion of one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more) nucleotides. In certain embodiments, the promoter sequence of the endogenous mogroside synthesis pathway gene is modified by replacement of the promoter sequence with one or more substitutes. In particular, the substitute can be a cisgenic substitute. For example, the promoter sequence of the gene can be modified by replacement of the promoter sequence with one or more cisgenic substitutes. Alternatively, the substitute can be a transgenic substitute. For example, the promoter sequence of the endogenous gene can be modified by replacement of the promoter sequence with one or more transgenic substitutes.

In certain embodiments, the promoter sequence of the endogenous mogroside synthesis pathway gene is modified by correction of the promoter sequence. A promoter sequence may be corrected by deletion, modification, and/or correction of one or more polymorphisms or mutations that would otherwise limit the activity of the promoter sequence. In particular, the promoter sequence of the endogenous gene can be modified by: (i) detection of one or more polymorphism or mutation that limits the activity of the promoter sequence; and (ii) correction of the promoter sequences by deletion, modification, and/or correction of the polymorphism or mutation.

In certain embodiments, the promoter sequence of the endogenous mogroside synthesis pathway gene is modified by insertion, deletion, and/or modification of one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more) upstream nucleotide sequences. In particular, the activity of the promoter sequence of the endogenous gene can be enhanced, improved, and/or increased by insertion, deletion, and/or modification of one or more upstream nucleotide sequences.

In certain embodiments, the promoter sequence of the endogenous mogroside synthesis pathway gene is modified by addition, insertion, and/or engineering of cis-acting factors. For example, the activity of the promoter sequence of the endogenous gene can be enhanced, improved, and/or increased by addition, insertion, and/or engineering of cis-acting factors that interact with and modify the promoter sequence.

In some embodiments, genetically-modifying the genome of a plant cell or plant part to modulate (e.g., increase or decrease) the expression of one or more endogenous mogroside synthesis pathway genes encompasses modulation (e.g., increase or decrease) of expression of one or more transcription factor genes. For example, to increase the expression of one or more endogenous mogroside synthesis pathway genes, the genome of a plant cell or plant part can be genetically-modified by modulation (e.g., increase or decrease) of expression of one or more transcription factor genes. In certain embodiments, the modulation of expression of the one or more transcription factor genes activates promoter sequences of the one or more endogenous mogroside synthesis pathway genes. In specific embodiments of the method, the modulation of expression of the one or more transcription factor genes increases the expression of one or more endogenous mogroside synthesis pathway genes.

In some embodiments, modulation of the expression of one or more endogenous mogroside synthesis pathway genes encompasses insertion, modification, and/or engineering of transcription factor binding sites or enhancer elements. In certain embodiments, modulation of the expression of the one or more endogenous mogroside synthesis pathway genes encompasses insertion of novel transcription factor binding sites or enhancer elements. Alternatively, modulation of the expression of the one or more endogenous mogroside synthesis pathway genes may encompass modification and/or engineering of existing transcription factor binding sites or enhancer elements.

(ii) Modification of Negative Regulatory Sequence

In some embodiments of the present disclosure, genetically-modifying the genome of a plant cell or plant part to modulate (e.g., increase or decrease) the expression of one or more endogenous mogroside synthesis pathway genes encompasses modification (e.g., insertion or deletion, such as insertion or deletion of part or whole) of one or more negative regulatory sequences of the genes. For example, to increase the expression of one or more endogenous mogroside synthesis pathway genes, the genome of a plant cell or plant part can be genetically-modified by modifying one or more negative regulatory sequences of the genes. In certain embodiments, the negative regulatory sequence of the gene is in a cis location. Alternatively, the negative regulatory sequence of the gene may be in a trans location. In particular embodiments, the negative regulatory sequences include upstream open reading frames (uORFs). For example, a region upstream of an endogenous mogroside synthesis pathway gene or a region upstream of an endogenous promoter of a mogroside synthesis pathway gene can be deleted in order to modulate expression of the mogroside synthesis pathway gene. In some embodiments, the region upstream of the endogenous mogroside synthesis pathway gene or promoter to be modified or deleted is at least 1, 2, 3, 4, 5, 10, 15, 20, 25, 30, 35, 40, 45, 50, 55, 60, 70, 80, 90, 100, 150, 200, 250, or more nucleotides in length or at least 1-10, 10-20, 20-30, 30-40, 40-50, 50-60, 60-70, 70-80, 80-90, 90-100, 100-150, 150-200, 200-250, 250-300, 300-400, 400-500, 500-750, or 750-1000 nucleotides in length. Further, the region upstream of the endogenous mogroside synthesis pathway gene or promoter to be modified or deleted can be about 1, 2, 3, 4, 5, 10, 15, 20, 25, 30, 35, 40, 45, 50, 55, 60, 70, 80, 90, 100, 150, 200, 250, or more nucleotides upstream of the gene or promoter or about 1-10, 10-20, 20-30, 30-40, 40-50, 50-60, 60-70, 70-80, 80-90, 90-100, 100-150, 150-200, 200-250, 250-300, 300-400, 400-500, 500-750, or 750-1000 nucleotides upstream of the gene or promoter.

(iii) Insertion of Functional Promoter

In some instances, genetically-modifying the genome of a plant cell or plant part to modulate the expression of one or more endogenous mogroside synthesis pathway genes encompasses insertion of one or more functional promoters into the genome of the cell or plant part, wherein the functional promoters are operably linked to the respective endogenous mogroside synthesis pathway gene following the insertion. For example, to modulate the expression of one or more endogenous mogroside synthesis pathway genes, the genome of a plant cell or plant part can be genetically-modified by insertion of one or more functional promoters into the genome of the cell or plant part, wherein the functional promoters are operably linked to the respective gene following the insertion. In certain embodiments, the one or more functional promoters are homologous promoters. For example, to modulate the expression of the one or more endogenous mogroside synthesis pathway genes, the genome of a plant cell or plant part can be genetically-modified by insertion of one or more homologous or cis-genic promoters into the genome of the cell or plant part, wherein the homologous or cis-genic promoters are operably linked to the one or more endogenous mogroside synthesis pathway genes following the insertion. In other embodiments, the one or more functional promoters are heterologous promoters. For example, to modulate the expression of the one or more endogenous mogroside synthesis pathway genes, the genome of a plant cell or plant part can be genetically-modified by insertion of one or more heterologous promoters into the genome of the cell or plant part, wherein the heterologous promoters are operably linked to the one or more mogroside synthesis pathway genes following the insertion.

(C) Expression Constructs

Also described herein are expression constructs that may be used for modulating the expression of one or more endogenous mogroside synthesis pathway genes in plants disclosed herein. In some embodiments, the expression construct contains a cis-acting regulatory element that is operably linked to the one or more mogroside synthesis pathway genes. In certain embodiments, the cis-acting regulatory element directs and/or modulates the expression of the one or more endogenous mogroside synthesis pathway genes. For example, an expression construct may contain a cis-acting regulatory element that is operably linked to one or more mogroside synthesis pathway genes, wherein the cis-acting regulatory element may modulate the expression of the one or more endogenous mogroside synthesis pathway genes in a cell. In particular embodiments, the cis-acting regulatory element is a promoter. In specific embodiments, an expression construct may contain a promoter sequence that is operably linked to one or more mogroside synthesis pathway genes, wherein the promoter sequence may modulate the expression of the one or more mogroside synthesis pathway genes (e.g., one or more endogenous mogroside synthesis pathway genes) in a cell in order to increase the level of mogrosides in the cell. In certain instances, an expression construct may contain a cassava vein mosaic virus (CSVMV) promoter that is operably linked to one or more mogroside synthesis pathway genes (e.g., one or more endogenous mogroside synthesis pathway genes), wherein the promoter sequence may modulate (e.g., increase or decrease) the expression of the one or more mogroside synthesis pathway genes in a cell in order to increase the level of mogrosides in the cell.

An expression construct disclosed herein may contain a promoter sequence operably linked to a gene (e.g., an endogenous gene) encoding a polypeptide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth in any one of SEQ ID NOs: 1, 2, 3, 4, 5, 6, 7, 8, 9, 10, 11, 12, 13, 14, 15, 16, 17, 18, 19, 20, or 21. For example, an expression construct disclosed herein may contain a CSVMV promoter operably linked to a gene (e.g., an endogenous gene) encoding a polypeptide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence set forth in any one of SEQ ID NOs: 1, 2, 3, 4, 5, 6, 7, 8, 9, 10, 11, 12, 13, 14, 15, 16, 17, 18, 19, 20, or 21.

In some embodiments, an expression construct disclosed herein may contain a promoter sequence, a leader sequence, and/or one or more nuclease recognition sites. In certain embodiments, an expression construct disclosed herein may be a repair template, such as a repair template containing a promoter (e.g., a viral promoter, such as a CsVMV promoter) and a leader sequence (e.g., a SynJ 5′ leader sequence) inserted between a pair of nuclease recognition sites (e.g., a pair of engineered meganuclease cleavage sites).

In some embodiments, an expression construct described herein may contain additional regulatory signals, including, but not limited to, transcriptional initiation start sites, operators, activators, enhancers, other regulatory elements, ribosomal binding sites, an initiation codon, termination signals, and the like. See, for example, U.S. Pat. Nos. 5,039,523 and 4,853,331; EPO 0480762A2; Sambrook et al. (1992) Molecular Cloning: A Laboratory Manual, ed. Maniatis et al. (Cold Spring Harbor Laboratory Press, Cold Spring Harbor, N.Y.), hereinafter “Sambrook 11”; Davis et al., eds. (1980) Advanced Bacterial Genetics (Cold Spring Harbor Laboratory Press), Cold Spring Harbor, N.Y., and the references cited therein.

In preparing the expression cassette, various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the present disclosure. The promoters can be selected based on the desired outcome. The nucleic acids can be combined with constitutive, inducible, tissue-preferred, or other promoters for expression in the organism of interest. See, for example, promoters set forth in WO 99/43838 and in U.S. Pat. Nos. 8,575,425; 7,790,846; 8,147,856; 8,586832; 7,772,369; 7,534,939; 6,072,050; 5,659,026; 5,608,149; 5,608,144; 5,604,121; 5,569,597; 5,466,785; 5,399,680; 5,268,463; 5,608,142; and 6,177,611; herein incorporated by reference.

For expression in plants, constitutive promoters also include CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appi. Genet. 81:581-588); MA S (Velten et al. (1984) EMBO 1 3:2723-2730).

In specific embodiments endogenous promoters of the mogroside synthesis pathway genes can be used in the methods and compositions disclosed herein. Any endogenous promoters of the mogroside synthesis pathway can be moved to modulate the expression of any endogenous gene of the mogroside synthesis pathway that is heterologous to the promoter. For example, the promoter of the CDS, CYP87D18, EPH3, SQE, UGT720, or UGT94 genes can be used to modulate the expression of the CDS, CYP87D18, EPH3, SQE, UGT720, or UGT94 genes other than the gene native to the promoter. In some embodiments, the native promoter of an endogenous mogroside synthesis pathway gene used to modulate the expression of another endogenous mogroside synthesis pathway gene can be modified to modulate activity of the promoter. In some embodiments, the native promoter of a mogroside synthesis pathway gene of one Cucurbitaceae family plant can be operably linked to an endogenous mogroside synthesis pathway gene of another Cucurbitaceae family plant. For example, the native promoter of a mogroside synthesis pathway gene of any Cucurbitaceae family plant can be operably linked to an endogenous mogroside synthesis pathway gene of a watermelon or cucumber plant in order to modulate the expression of an endogenous mogroside synthesis pathway gene in the watermelon or cucumber plant.

In particular embodiments, any endogenous promoter from the genome of a Cucurbitaceae family plant can be used to modulate the expression of an endogenous mogroside synthesis pathway gene. For example, any promoter from the genome of a watermelon plant can be used to modulate the expression of an endogenous mogroside synthesis pathway gene of the watermelon genome. Likewise, any promoter from the genome of a cucumber plant can be used to modulate the expression of an endogenous mogroside synthesis pathway gene of the cucumber genome.

Tissue-preferred promoters for use in the invention include those set forth in Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probi. Cell Differ. 20:181-196; Orozco et al. (1993) Plant Mol Biol. 23(6): 1129-1138; Matsuoka et al. (1993) Proc Natl. Acad. Sci. USA 90(20):9586-9590; and Guevara-Garcia et al. (1993) Plant J. 4(3):495-505.

Leaf-preferred promoters include those set forth in Yamamoto et al. (1997) PlantJ. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mal. Biol. 23(6):1129-1138; and Matsuoka et al. (1993) Proc. Natl. Acad. Sci. USA 90(20):9586-9590.

Root-preferred promoters are known and include those in Hire et al. (1992) Plant Ma Biol. 20(2):207-218 (soybean root-specific glutamine synthetase gene); Keller and Baumgartner (1991) Plant Cell 3(10):1051-1061 (root-specific control element); Sanger et al. (1990) Plant Mol. Biol. 14(3):433-443 (mannopine synthase (MAS) gene of Agrobacterium tumefiiciens); and Miao et al. (1991) Plant Cell 3(1):11-22 (cytosolic glutamine synthetase (GS)); Bogusz et al. (1990) Plant Cell 2(7):633-641; Leach and Aoyagi (1991) Plant Science (Limerick) 79(1):69-76 (roIC and roID); Teeri et al. (1989) EIVIBO J. 8(2):343-350; Kuster et al. (1995) Plant Mot Biol. 29(4):759-772 (the VfENOD-GRP3 gene promoter); and, Capana et al. (1994) Plant Mol. Biol. 25(4):681-691 (roIB promoter). See also U.S. Pat. Nos. 5,837,876; 5,750,386; 5,633,363; 5,459,252; 5,401,836; 5,110,732; and 5,023,179.

“Seed-preferred” promoters include both “seed-specific” promoters (those promoters active during seed development such as promoters of seed storage proteins) as well as “seed-germinating” promoters (those promoters active during seed germination). See Thompson et al. (1989) BioEssays 10:108. Seed-preferred promoters include, but are not limited to, Ciml (cytokinin-induced message); cZ19B1 (maize 19 kDa zein); milps (myo-inositol-l-phosphate synthase) (see WO 00/11177 and U.S. Pat. No. 6,225,529). Gamma-zein is an endosperm-specific promoter. Globulin 1 (Glb-1) is a representative embryo-specific promoter. For dicots, seed-specific promoters include, but are not limited to, bean P-phaseolin, napin, P-conglycinin, soybean lectin, cruciferin, and the like. For monocots, seed-specific promoters include, but are not limited to, maize 15 kDa zein, 22 kDa zein, 27 kDa zein, gamma-zein, waxy, shrunken 1, shrunken 2, Globulin 1, etc. See also WO 00/12733, where seed-preferred promoters from end1 and end2 genes are disclosed.

### 2.4 Composition Containing Mogrosides

The methods and compositions described herein further encompass compositions containing mogrosides (e.g., mogrol, mogroside I-Al, mogroside I-El, mogroside IIA, mogroside IIB, mogroside IIE, 7-oxomogroside IIE, 11-oxomogroside A1, mogroside III, mogroside III A1, mogroside III A2, mogroside IIIx, 11-deoxymogroside III, mogroside IV, mogroside IV-A, 11-oxomogroside IV-A, siamenoside I, mogroside V, 7-oxomogroside V, 11-oxomogroside V, mogroside VI, and/or mogroside VII). For example, the polypeptides, polynucleotides, cells, and methods of the present disclosure can be used to produce at least one mogroside or a composition containing at least one mogroside. In certain embodiments, a composition described herein contains extract (e.g., sweetener), plant parts (e.g., juice, pulp, seed, fruit, flowers, embryos, pollen, ovules, leaves, branches, kernels, ears, cobs, husks, stalks, roots, root tips, anthers, etc.), plant concentrate (e.g., whole plant concentrate or plant part concentrate), plant biomass (e.g., dried biomass, such as crushed and/or powdered biomass), and/or plant powder (e.g., formulated powder, such as formulated plant part powder) of the present disclosure, wherein the extract, plant part, plant concentrate, plant biomass, and/or plant powder has increased levels of one or more mogrosides.

In certain embodiments, concentration of at least one mogrosides in a composition described herein is at least 0.2 ppm, 0.3 ppm, 0.4 ppm, 0.5 ppm, 0.6 ppm, 0.7 ppm, 0.8 ppm, 0.9 ppm, 1 ppm, 1.5 ppm, 2 ppm, 2.5 ppm, 3 ppm, 3.5 ppm, 4 ppm, 4.5 ppm, 5 ppm, 5.5 ppm, 6 ppm, 6.5 ppm, 7 ppm, 7.5 ppm, 8 ppm, 8.5 ppm, 9 ppm, 9.5 ppm, 10 ppm, 10.5 ppm, 11 ppm, 11.5 ppm, 12 ppm, 12.5 ppm, 13 ppm, 13.5 ppm, 14 ppm, 14.5 ppm, 15 ppm, 15.5 ppm, 16 ppm, 16.5 ppm, 17 ppm, 17.5 ppm, 18 ppm, 18.5 ppm, 19 ppm, 19.5 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 105 ppm, 110 ppm, 115 ppm, 120 ppm, 125 ppm, 130 ppm, 135 ppm, 140 ppm, 145 ppm, 150 ppm, 155 ppm, 160 ppm, 165 ppm, 170 ppm, 175 ppm, 180 ppm, 185 ppm, 190 ppm, 195 ppm, 200 ppm, 205 ppm, 210 ppm, 215 ppm, 220 ppm, 225 ppm, 230 ppm, 235 ppm, 240 ppm, 245 ppm, 250 ppm, 255 ppm, 260 ppm, 265 ppm, 270 ppm, 275 ppm, 280 ppm, 285 ppm, 290 ppm, 295 ppm, 300, 305 ppm, 310 ppm, 315 ppm, 320 ppm, 325 ppm, 330 ppm, 335 ppm, 340 ppm, 345 ppm, 350 ppm, 355 ppm, 360 ppm, 365 ppm, 370 ppm, 375 ppm, 380 ppm, 385 ppm, 390 ppm, 395 ppm, 400 ppm, 405 ppm, 410 ppm, 415 ppm, 420 ppm, 425 ppm, 430 ppm, 435 ppm, 440 ppm, 445 ppm, 450 ppm, 455 ppm, 460 ppm, 465 ppm, 470 ppm, 475 ppm, 480 ppm, 485 ppm, 490 ppm, 495 ppm, 500 ppm, 525 ppm, 550 ppm, 600 ppm, 625 ppm, 650 ppm, 700 ppm, 725 ppm, 750 ppm, 800 ppm, 825 ppm, 850 ppm, 900 ppm, 925 ppm, 950 ppm, 1000 ppm, or more.

In certain embodiments, the concentration of mogrosides in a composition described herein is at least 10% (wt/wt), 15% (wt/wt), 20% (wt/wt), 25% (wt/wt), 30% (wt/wt), 35% (wt/wt), 40% (wt/wt), 45% (wt/wt), 50% (wt/wt), 55% (wt/wt), 60% (wt/wt), 65% (wt/wt), 70% (wt/wt), 75% (wt/wt), 80% (wt/wt), 85% (wt/wt), 90% (wt/wt), 95% (wt/wt), 97% (wt/wt), 99% (wt/wt), or more.

In some embodiments, a composition described herein, such as a composition containing extract, plant part, plant concentrate, plant biomass, and/or plant powder of the present disclosure that has increased levels of one or more mogrosides is a consumable. In some embodiments, the composition, such as composition containing extract, plant part, plant concentrate, plant biomass, and/or plant powder of the present disclosure that has increased levels of one or more mogrosides is used as an ingredient in one or more consumables. The composition disclosed herein, such as composition containing extract, plant part, plant concentrate, plant biomass, and/or plant powder of the present disclosure that has increased levels of one or more mogrosides can be used as an additive in one or more consumables. Consumables may include all food products, including but not limited to, cereal products, rice products, tapioca products, sago products, baker's products, biscuit products, pastry products, bread products, confectionery products, desert products, gums, chewing gums, chocolates, ices, honey products, treacle products, yeast products, baking-powder, salt and spice products, savory products, mustard products, vinegar products, sauces (condiments), tobacco products, cigars, cigarettes, processed foods, cooked fruits and vegetable products, meat and meat products, jellies, jams, fruit sauces, egg products, milk and dairy products, yogurts, cheese products, butter and butter substitute products, milk substitute products, soy products, edible oils and fat products, medicaments, beverages, carbonated beverages, alcoholic drinks, beers, soft drinks, mineral and aerated waters and other non-alcoholic drinks, fruit drinks, fruit juices, coffee, artificial coffee, tea, cocoa, including forms requiring reconstitution, food extracts, plant extracts, meat extracts, condiments, sweeteners, nutraceuticals, gelatins, pharmaceutical and non-pharmaceutical gums, tablets, lozenges, drops, emulsions, elixirs, syrups and other preparations for making beverages, and combinations thereof. A composition described herein can be used in various consumables including but not limited to water-based consumables, solid dry consumables, dairy products, dairy-derived products, and dairy-alternative products. In some embodiments, the composition is a foodstuff.

Water-based consumables include but are not limited to beverage, water, aqueous drink, enhanced/slightly sweetened water drink, mineral water, carbonated beverage, non-carbonated beverage, carbonated water, still water, soft drink, non-alcoholic drink, alcoholic drink, beer, wine, liquor, fruit drink, juice, fruit juice, vegetable juice, broth drink, coffee, tea, black tea, green tea, oolong tea, herbal tea, cacao (water-based), tea-based drink, coffee-based drink, cacao-based drink, syrup, frozen fruit, frozen fruit juice, water-based ice, fruit ice, sorbet, dressing, salad dressing, sauce, soup, and beverage botanical materials (whole or ground), or instant powder for reconstitution (coffee beans, ground coffee, instant coffee, cacao beans, cacao powder, instant cacao, tea leaves, instant tea powder).

In certain embodiments, a composition described herein (e.g., composition containing mogrosides) can be used in a water-based consumable, such as a beverage. In particular, the beverage may be selected from the group including an aqueous beverage, enhanced/slightly sweetened water drink, mineral water, carbonated beverage, non-carbonated beverage, carbonated water, still water, soft drink, non-alcoholic drink, alcoholic drink, beer, wine, liquor, fruit drink, juice, fruit juice, vegetable juice, broth drink, coffee, tea, black tea, green tea, oolong tea, herbal tea, cacao, tea-based drink, coffee-based drinks, cacao-based drink, syrup, dairy products, frozen fruit, frozen fruit juice, water-based ice, fruit ice, sorbet, dressing, salad dressing, sauce, soup, and beverage botanical materials, or instant powder for reconstitution.

In certain embodiments, a composition described herein can be used in a solid dry consumable. Solid dry consumables include but are not limited to cereals, baked food products, biscuits, bread, breakfast cereal, cereal bar, energy bars/nutritional bars, granola, cakes, cookies, crackers, donuts, muffins, pastries, confectioneries, chewing gum, chocolate, fondant, hard candy, marshmallow, pressed tablets, snack foods, and botanical materials (whole or ground), and instant powders for reconstitution as mentioned above.

For use in water-based consumables or solid dry consumables, a useful concentration of mogrosides may be from about 0.2-300 ppm, such as about 0.2 ppm, 0.3 ppm, 0.4 ppm, 0.5 ppm, 0.6 ppm, 0.7 ppm, 0.8 ppm, 0.9 ppm, 1 ppm, 1.5 ppm, 2 ppm, 2.5 ppm, 3 ppm, 3.5 ppm, 4 ppm, 4.5 ppm, 5 ppm, 5.5 ppm, 6 ppm, 6.5 ppm, 7 ppm, 7.5 ppm, 8 ppm, 8.5 ppm, 9 ppm, 9.5 ppm, 10 ppm, 10.5 ppm, 11 ppm, 11.5 ppm, 12 ppm, 12.5 ppm, 13 ppm, 13.5 ppm, 14 ppm, 14.5 ppm, 15 ppm, 15.5 ppm, 16 ppm, 16.5 ppm, 17 ppm, 17.5 ppm, 18 ppm, 18.5 ppm, 19 ppm, 19.5 ppm, 20 ppm, 25 ppm, 30 ppm, 35 ppm, ppm, 45 ppm, 50 ppm, 55 ppm, 60 ppm, 65 ppm, 70 ppm, 75 ppm, 80 ppm, 85 ppm, 90 ppm, 95 ppm, 100 ppm, 105 ppm, 110 ppm, 115 ppm, 120 ppm, 125 ppm, 130 ppm, 135 ppm, 140 ppm, 145 ppm, 150 ppm, 155 ppm, 160 ppm, 165 ppm, 170 ppm, 175 ppm, 180 ppm, 185 ppm, 190 ppm, 195 ppm, 200 ppm, 205 ppm, 210 ppm, 215 ppm, 220 ppm, 225 ppm, 230 ppm, 235 ppm, 240 ppm, 245 ppm, 250 ppm, 255 ppm, 260 ppm, 265 ppm, 270 ppm, 275 ppm, 280 ppm, 285 ppm, 290 ppm, 295 ppm, or 300 ppm.

In certain consumables, a higher sweetener concentration may be necessary to reach similar sweetness intensity; for example, for use of a composition of the present disclosure in dairy products, dairy-derived products and dairy-alternative products. Dairy-derived food products contain milk or milk protein. Dairy-alternative products contain (instead of dairy protein derived from the milk of mammals) protein from plant sources (e.g., soy, rice, almond, and other protein-rich plant materials). Dairy products, dairy-derived products, and dairy-alternative products include but are not limited to milk, fluid milk, cultured milk product, cultured and non-cultured dairy-based drinks, cultured milk product cultured with lactobacillus, yogurt, yogurt-based beverage, smoothie, lassi, milk shake, acidified milk, acidified milk beverage, butter milk, kefir, milk-based beverage, milk/juice blend, fermented milk beverage, ice cream, dessert, sour cream, dip, salad dressings, cottage cheese, frozen yogurt, soy milk, almond milk, rice milk, soy drink, rice milk drink, etc. Milk includes, but is not limited to, whole milk, skim milk, condensed milk, evaporated milk, reduced fat milk, low fat milk, nonfat milk, and milk solids (which may be fat or nonfat).

A useful concentration of mogrosides for dairy products, dairy-derived products and dairy-alternative products, may be about 0.3-500 ppm or higher, and may be up to 550 ppm, 600 ppm, 650 ppm, 700 ppm, 750 ppm or more, such as about 0.3 ppm, 0.4 ppm, 0.5 ppm, 0.6 ppm, 0.7 ppm, 0.8 ppm, 0.9 ppm, 1 ppm, 1.5 ppm, 2 ppm, 2.5 ppm, 3 ppm, 3.5 ppm, 4 ppm, 4.5 ppm, 5 ppm, 5.5 ppm, 6 ppm, 6.5 ppm, 7 ppm, 7.5 ppm, 8 ppm, 8.5 ppm, 9 ppm, 9.5 ppm, 10 ppm, 10.5 ppm, 11 ppm, 11.5 ppm, 12 ppm, 12.5 ppm, 13 ppm, 13.5 ppm, 14 ppm, 14.5 ppm, 15 ppm, 15.5 ppm, 16 ppm, 16.5 ppm, 17 ppm, 17.5 ppm, 18 ppm, 18.5 ppm, 19 ppm, 19.5 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 105 ppm, 110 ppm, 115 ppm, 120 ppm, 125 ppm, 130 ppm, 135 ppm, 140 ppm, 145 ppm, 150 ppm, 155 ppm, 160 ppm, 165 ppm, 170 ppm, 175 ppm, 180 ppm, 185 ppm, 190 ppm, 195 ppm, 200 ppm, 205 ppm, 210 ppm, 215 ppm, 220 ppm, 225 ppm, 230 ppm, 235 ppm, 240 ppm, 245 ppm, 250 ppm, 255 ppm, 260 ppm, 265 ppm, 270 ppm, 275 ppm, 280 ppm, 285 ppm, 290 ppm, 295 ppm, 300 ppm, 305 ppm, 310 ppm, 315 ppm, 320 ppm, 325 ppm, 330 ppm, 335 ppm, 340 ppm, 345 ppm, 350 ppm, 355 ppm, 360 ppm, 365 ppm, 370 ppm, 375 ppm, 380 ppm, 385 ppm, 390 ppm, 395 ppm, 400 ppm, 405 ppm, 410 ppm, 415 ppm, 420 ppm, 425 ppm, 430 ppm, 435 ppm, 440 ppm, 445 ppm, 450 ppm, 455 ppm, 460 ppm, 465 ppm, 470 ppm, 475 ppm, 480 ppm, 485 ppm, 490 ppm, 495 ppm, 500 ppm, 525 ppm, 550 ppm, 600 ppm, 625 ppm, 650 ppm, 700 ppm, 725 ppm, 750 ppm, or more.

In some embodiments, concentration of mogrosides in the composition described herein (e.g., a composition containing extract, plant part, plant concentrate, plant biomass, and/or plant powder of the present disclosure that has increased levels of one or more mogrosides) is sufficient to cause an enhancement in flavor. In some embodiments, wherein an enhanced sweetness is desired, concentration of mogrosides in the composition is sufficient to cause an enhancement in flavor, and can be used as a sweetener. In certain embodiments, a composition disclosed herein, such as a composition containing extract, plant part, plant concentrate, plant biomass, and/or plant powder of the present disclosure that has increased levels of one or more mogrosides is a sweetener. Such a composition can contain a concentration of mogrosides of at least 0.2 ppm, such as at least 0.2-100 ppm, 0.2-200 ppm, 0.2-300 ppm, 0.2-400 ppm, 0.2-500 ppm, or at least about 0.2 ppm, 0.3 ppm, 0.4 ppm, 0.5 ppm, 0.6 ppm, 0.7 ppm, 0.8 ppm, 0.9 ppm, 1 ppm, 1.5 ppm, 2 ppm, 2.5 ppm, 3 ppm, 3.5 ppm, 4 ppm, 4.5 ppm, 5 ppm, 5.5 ppm, 6 ppm, 6.5 ppm, 7 ppm, 7.5 ppm, 8 ppm, 8.5 ppm, 9 ppm, 9.5 ppm, 10 ppm, 10.5 ppm, 11 ppm, 11.5 ppm, 12 ppm, 12.5 ppm, 13 ppm, 13.5 ppm, 14 ppm, 14.5 ppm, 15 ppm, 15.5 ppm, 16 ppm, 16.5 ppm, 17 ppm, 17.5 ppm, 18 ppm, 18.5 ppm, 19 ppm, 19.5 ppm, 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, 85 ppm, 90 ppm, 95 ppm, 100 ppm, 105 ppm, 110 ppm, 115 ppm, 120 ppm, 125 ppm, 130 ppm, 135 ppm, 140 ppm, 145 ppm, 150 ppm, 155 ppm, 160 ppm, 165 ppm, 170 ppm, 175 ppm, 180 ppm, 185 ppm, 190 ppm, 195 ppm, 200 ppm, 205 ppm, 210 ppm, 215 ppm, 220 ppm, 225 ppm, 230 ppm, 235 ppm, 240 ppm, 245 ppm, 250 ppm, 255 ppm, 260 ppm, 265 ppm, 270 ppm, 275 ppm, 280 ppm, 285 ppm, 290 ppm, 295 ppm, 300 ppm, 305 ppm, 310 ppm, 315 ppm, 320 ppm, 325 ppm, 330 ppm, 335 ppm, 340 ppm, 345 ppm, 350 ppm, 355 ppm, 360 ppm, 365 ppm, 370 ppm, 375 ppm, 380 ppm, 385 ppm, 390 ppm, 395 ppm, 400 ppm, 405 ppm, 410 ppm, 415 ppm, 420 ppm, 425 ppm, 430 ppm, 435 ppm, 440 ppm, 445 ppm, 450 ppm, 455 ppm, 460 ppm, 465 ppm, 470 ppm, 475 ppm, 480 ppm, 485 ppm, 490 ppm, 495 ppm, 500 ppm, or more.

In some embodiments, a composition disclosed herein further includes a flavor ingredient selected from the group including sucrose, fructose, glucose, high fructose corn syrup, xylose, arabinose, rhamnose, erythritol, xylitol, mannitol, sorbitol, inositol, AceK, aspartame, neotame, sucralose, saccharine, naringin dihydrochalcone (NarDHC), neohesperidin dihydrochalcone (NDHC), rubusoside, rebaudioside A, stevioside, stevia, and/or trilobtain.

The compositions disclosed herein comprising at least one mogroside produced from plants with modulated expression of at least one endogenous gene of the mogroside synthesis pathway can include one or more additional flavor ingredients, such as additional sweeteners. A non-limiting list of suitable flavor ingredients useful with the composition of the present disclosure includes sucrose, fructose, glucose, high fructose corn syrup, xylose, arabinose, rhamnose, erythritol, xylitol, mannitol, sorbitol, inositol, AceK, aspartame, neotame, sucralose, saccharine, naringin dihydrochalcone (NarDHC), neohesperidin dihydrochalcone (NDHC), rubusoside, rebaudioside A, stevioside, stevia and trilobtain.

Sweeteners commonly used in consumables include:

Acesulfame K—Artificial Sweetener (E950); Agave Syrup—Modified Sugar; Alitame—Artificial Sweetener (E956); Aspartame—Artificial Sweetener (E951); Aspartame-Acesulfame Salt—Artificial Sweetener (E962); Barley Malt Syrup—Modified Sugar; Birch Syrup—Sugar Extract; Blackstrap Molasses—Sugar Extract; Brazzein—Natural Sweetener; Brown Rice Syrup—Modified Sugar; Cane Juice—Sugar Extract; Caramel—Modified sugar; Coconut Palm Sugar—Sugar Extract; Corn Sugar (HFCS)—Modified sugar; Corn Sweetener (HFCS)— Modified sugar; Corn Syrup (HFCS)—Modified sugar; Curculin—Natural Sweetener; Cyclamate—Artificial Sweetener (E952); Dextrose—Sugar; Erythritol—Sugar Alcohol (E968); Fructose Glucose Syrup (HFCS)—Modified sugar; Fructose—Sugar; Galactose—Sugar; Glucitol (Sorbitol)—Sugar Alcohol (E420); Glucose—Sugar; Glucose Fructose Syrup (HFCS)—Modified sugar; Glycerol (Glycerin)—Sugar Alcohol (E422); Glycyrrhizin—Natural Sweetener (E958); Golden Syrup—Modified sugar; High Fructose Corn Syrup (HFCS)—Modified Sugar; HFCS-42—Modified Sugar; HFCS-55—Modified Sugar; HFCS-90—Modified Sugar; Honey—Natural Sugar; HSH—Sugar Alcohol; Hydrogenated Starch Hydrolysate (HSH)—Sugar Alcohol; Isoglucose (HFCS)— Modified sugar; Inulin—Sugar Fiber; Inverted Sugar—Modified sugar; Isomalt—Sugar Alcohol (E953); Lactitol—Sugar Alcohol (E966); Lactose—Sugar; Levulose (Fructose)—Sugar; Luo Han Guo—Natural Sweetener; Maltitol—Sugar Alcohol (E965); Maltodextrin—Sugar; Maltose—Sugar; Mannitol—Sugar Alcohol (E421); Maple Syrup—Sugar Extract; Miraculin—Natural Sweetener; Molasses—Sugar Extract; Monellin—Natural Sweetener; Monk Fruit (Luo Han Guo)—Natural Sweetener; Neohesperidin DC—Artificial Sweetener (E959); Neotame—Artificial Sweetener (E961); Oligofructose—Sugar Fiber; Palm Sugar—Sugar Extract; Pentadin—Natural Sweetener; Rapadura—Sugar Extract; Refiners Syrup—Modified Sugar; Saccharin—Artificial Sweetener (E954); Saccharose (Sucrose)—Sugar; Sorbitol—Sugar Alcohol (E420); Sorghum Syrup—Sugar Extract; Stevia—Natural Sweetener; Stevioside—Natural Sweetener (E960); Sucralose—Artificial Sweetener (E955); Sucrose—Sugar; Tagatose—Modified Sugar; Thaumatin—Natural Sweetener (E957); Trehalose—Sugar; Xylitol—Sugar Alcohol (E967); Yacon Syrup—Natural Sweetener.

In some embodiments, a composition described herein comprising at least one mogroside produced from plants with modulated expression of at least one endogenous gene of the mogroside synthesis pathway is a therapeutic. In some embodiments, the composition is used as an ingredient or additive in one or more therapeutics. In certain embodiments, the therapeutic has a health-promotion function and/or a disease prevention function. In specific embodiments, the therapeutic is a functional food, such as a food having one or more additional functions (e.g., a health-promotion function and/or a disease prevention function). Additionally, or alternatively, the therapeutic may be a pill, a tablet, a powder, a gel, an emulsion, a liquid, a gum, lozenge, drop, elixir, and/or a syrup with a health-promotion function and/or a disease prevention function. In some embodiments, a composition described herein comprising at least one mogroside produced from plants with modulated expression of at least one gene of the mogroside synthesis pathway is a prebiotic to induce the growth or activity of beneficial microorganisms, such as bacteria and fungi (e.g., in the gastrointestinal tract of a human subject, where the prebiotic can alter the composition of microorganisms in the gut microbiome).

### 2.5 Transformation of Plants

Also disclosed herein are methods for transforming a plant, such as a cell of a plant (e.g., a Cucurbitaceae family plant, such as a watermelon (e.g., a Charleston Gray variety watermelon) plant or a cucumber plant) of the present disclosure with a nucleic acid in order to modulate the expression of at least one endogenous mogroside synthesis pathway gene and increase the level of at least one mogroside in the cell.

Plant cells may be transformed stably or transiently with the nucleic acid constructs (e.g., expression cassette) of some embodiments of the disclosure. In stable transformation, the nucleic acid molecule of some embodiments of the disclosure is integrated into the plant genome and as such it represents a stable and inherited trait. In transient transformation, the nucleic acid molecule is expressed by the cell transformed but it is not integrated into the genome and as such it represents a transient trait.

There are various methods of introducing foreign genes into both monocotyledonous and dicotyledonous plants (Potrykus et al., Annu Rev Plant Physiol Plant Mol Biol 42:205-225 (1991); Shimamoto et al., Nature 338:274-276 (1989)).

The principle methods of causing stable integration of exogenous DNA into plant genomic DNA include two main approaches:

(i) Agrobacterium-mediated gene transfer: Klee et al., Annu Rev Plant Physiol 38:467-486 (1987); Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112.

(ii) direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; glass fibers or silicon carbide whisker transformation of cell cultures, embryos or callus tissue, U.S. Pat. No. 5,464,765 or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration.

The Agrobacterium system is especially viable in the creation of transgenic dicotyledonous plants.

There are various methods of direct DNA transfer into plant cells. In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or tungsten particles, and the microprojectiles are physically accelerated into cells or plant tissues.

Following stable transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the transformed plants.

Micropropagation is a process of growing new generation plants from a single piece of tissue that has been excised from a selected parent plant or cultivar. This process permits the mass reproduction of plants having the preferred tissue expressing the fusion protein. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

Although stable transformation is presently preferred, transient transformation of leaf cells, meristematic cells or the whole plant is also envisaged by some embodiments of the disclosure.

Transient transformation can be effected by any of the direct DNA transfer methods described above or by viral infection using modified plant viruses.

Viruses that have been shown to be useful for the transformation of plant hosts include CaMV, TMV, and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Construction of plant RNA viruses for the introduction and expression of non-viral exogenous nucleic acid sequences in plants is demonstrated by the above references as well as by Dawson, W. O. et al., Virology (1989) 172:285-292; Takamatsu et al. EMBO J. (1987) 6:307-311; French et al. Science (1986) 231:1294-1297; and Takamatsu et al. FEB S Letters (1990) 269:73-76.

When the virus is a DNA virus, suitable modifications can be made to the virus itself Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat protein which will encapsidate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Construction of plant RNA viruses for the introduction and expression in plants of non-viral exogenous nucleic acid sequences such as those included in the construct of some embodiments of the invention is demonstrated by the above references as well as in U.S. Pat. No. 5,316,931.

In one embodiment, a plant viral nucleic acid is provided in which the native coat protein coding sequence has been deleted from a viral nucleic acid, a non-native plant viral coat protein coding sequence and a non-native promoter, preferably the subgenomic promoter of the non-native coat protein coding sequence, capable of expression in the plant host, packaging of the recombinant plant viral nucleic acid, and ensuring a systemic infection of the host by the recombinant plant viral nucleic acid, has been inserted. Alternatively, the coat protein gene may be inactivated by insertion of the non-native nucleic acid sequence within it, such that a protein is produced. The recombinant plant viral nucleic acid may contain one or more additional non-native subgenomic promoters. Each non-native subgenomic promoter is capable of transcribing or expressing adjacent genes or nucleic acid sequences in the plant host and incapable of recombination with each other and with native subgenomic promoters. Non-native (foreign) nucleic acid sequences may be inserted adjacent the native plant viral subgenomic promoter or the native and a non-native plant viral subgenomic promoters if more than one nucleic acid sequence is included. The non-native nucleic acid sequences are transcribed or expressed in the host plant under control of the subgenomic promoter to produce the desired products.

In another embodiment, a recombinant plant viral nucleic acid is provided as in the first embodiment except that the native coat protein coding sequence is placed adjacent one of the non-native coat protein subgenomic promoters instead of a non-native coat protein coding sequence.

In a different embodiment, a recombinant plant viral nucleic acid is provided in which the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that the sequences are transcribed or expressed in the host plant under control of the subgenomic promoters to produce the desired product.

In an alternative embodiment, a recombinant plant viral nucleic acid is provided as in the third embodiment except that the native coat protein coding sequence is replaced by a non-native coat protein coding sequence.

The viral vectors are encapsidated by the coat proteins encoded by the recombinant plant viral nucleic acid to produce a recombinant plant virus. The recombinant plant viral nucleic acid or recombinant plant virus is used to infect appropriate host plants. The recombinant plant viral nucleic acid is capable of replication in the host, systemic spread in the host, and transcription or expression of foreign gene(s) (isolated nucleic acid) in the host to produce the desired protein.

In addition to the above, the nucleic acid molecule of some embodiments of the invention can also be introduced into a chloroplast genome thereby enabling chloroplast expression.

A technique for introducing exogenous nucleic acid sequences to the genome of the chloroplasts is known. This technique involves the following procedures. First, plant cells are chemically treated so as to reduce the number of chloroplasts per cell to about one. Then, the exogenous nucleic acid is introduced via particle bombardment into the cells with the aim of introducing at least one exogenous nucleic acid molecule into the chloroplasts. The exogenous nucleic acid is selected such that it is integratable into the chloroplast's genome via homologous recombination which is readily effected by enzymes inherent to the chloroplast. To this end, the exogenous nucleic acid includes, in addition to a gene of interest, at least one nucleic acid stretch which is derived from the chloroplast's genome. In addition, the exogenous nucleic acid includes a selectable marker, which serves by sequential selection procedures to ascertain that all or substantially all of the copies of the chloroplast genomes following such selection will include the exogenous nucleic acid. Further details relating to this technique are found in U.S. Pat. Nos. 4,945,050; and 5,693,507 which are incorporated herein by reference. A polypeptide can thus be produced by the protein expression system of the chloroplast and become integrated into the chloroplast's inner membrane. According to some embodiments of the invention, there is provided a host cell heterologously expressing an isolated polynucleotide of the invention, as described hereinabove. The host cell can be any suitable host cell include bacteria, yeast and other microorganisms that can be cultured or grown in fermentation, plant and other eukaryotic cells. For example, the host cell a bacterial cell (e.g., E. coli and B. subtilis) transformed with a heterologous nucleic acid, such as bacteriophage DNA, plasmid DNA, or cosmid DNA expression vectors containing the nucleic acid molecules described herein, or yeast (e.g., S. cerevisiae or S. pombe) transformed with recombinant yeast expression vectors containing the nucleic acid molecules described herein.

In some embodiments, the host cell is a yeast cell. In a specific embodiment, the yeast cell is a yeast cell deprived of endogenous sterol biosynthesis, such as GIL77, or a yeast line deficient in the endogenous squalene epoxidase ergl gene such as described in Rasbery J M et al. (Jour. Biol. Chem. 2007. 282:17002-17013).

In some embodiments, the host cell produces mogrol, mogrol or mogroside precursor, or mogroside. For example, the host cell may produce one or more of mogrol, mogroside I-Al, mogroside !-El, mogroside IIA, mogroside IIB, mogroside IIE, 7-oxomogroside IIE, 11-oxomogroside A1, mogroside III, mogroside III A1, mogroside III A2, mogroside Mx, 11-deoxymogroside III, mogroside IV, mogroside IV-A, 11-oxomogroside IV-A, siamenoside I, mogroside V, 7-oxomogroside V, 11-oxomogroside V, mogroside VI, and mogroside VII.

The methods may also employ a mixture of recombinant and non-recombinant host. If more than one host is used then the hosts may be co-cultivated, or they may be cultured separately. If the hosts are cultivated separately the intermediate products may be recovered and optionally purified and partially purified and fed to recombinant hosts using the intermediate products as substrates.

Recombinant hosts described herein can be used in methods to produce mogrosides. For example, if the recombinant host is a microorganism, the method can include growing the recombinant microorganism in a culture medium under conditions in which one or more of the enzymes catalyzing step(s) of the methods of the invention, e.g. synthases, hydrolases, CYP450s and/or UGTs are expressed. The recombinant microorganism may be grown in a fed batch or continuous process.

Typically, the recombinant microorganism is grown in a fermenter at a defined temperature(s) for a desired period of time. A cell lysate can be prepared from the recombinant host expressing one or more enzymes and be used to contact a substrate, such that mogrosides can be produced. For example, a cell lysate can be prepared from the recombinant host expressing one or more UGTs and used to contact mogrol or mogroside, such that mogrosides can be produced.

In some embodiments, mogrosides can be produced using whole cells that are fed raw materials that contain precursor molecules, e.g., mogrol. The raw materials may be fed during cell growth or after cell growth. The whole cells may be in suspension or immobilized. The whole cells may be in fermentation broth or in a reaction buffer. In some embodiments a permeabilizing agent may be required for efficient transfer of substrate into the cells.

Levels of products, substrates and intermediates can be determined by extracting samples from culture media for analysis according to published methods. Mogrosides can be recovered from the culture or culture medium using various techniques known in the art.

In some embodiments, there is provided a cell lysate of the host cell. Such a cell lysate can comprise both the mogroside pathway enzymes disclosed herein and the mogrol, mogrol and mogroside precursors and mogroside products of the pathways. Thus, the cell lysate can be used either for recovery of the products of the mogroside pathway (e.g. mogrol, mogroside M4, M5 and M6) or recovery of the recombinantly expressed enzymes polypeptides. Methods for extraction of active enzyme polypeptides are well known in the art.

Cell lysate of the disclosure can also be used for cell-free synthesis of mogrol, mogrol or mogroside precursors and mogroside, alone or in combination with other suitable substrates or enzymes.

In some embodiments, DNA constructs containing promoter sequences can be used to transform plants of interest or other organisms of interest. In certain embodiments, any expression cassette described herein can be used to genetically modify the genome of a plant in order to modulate expression of one or more endogenous mogroside synthesis pathway genes.

Methods for transformation involve introducing a nucleotide construct into a plant. By “introducing” is intended to introduce the nucleotide construct to the plant or other host cell in such a manner that the construct gains access to the interior of a cell of the plant or host cell. The methods of the present disclosure do not require a particular method for introducing a nucleotide construct to a plant or host cell, only that the nucleotide construct gains access to the interior of at least one cell of the plant or the host organism. Methods for introducing nucleotide constructs into plants and other host cells are known in the art including, but not limited to, stable transformation methods, transient transformation methods, and virus-mediated methods.

The methods result in a transformed organism, such as a plant, including whole plants, as well as plant organs (e.g., leaves, stems, roots, etc.), seeds, plant cells, propagules, embryos and progeny of the same. Plant cells can be differentiated or undifferentiated (e.g., callus, suspension culture cells, protoplasts, leaf cells, root cells, phloem cells, pollen).

As used herein, the term “transgenic” or “transformed” or “stably transformed” plants or cells or tissues refers to plants or cells or tissues that have been modified by the methods of the present disclosure to have a genetic modification that modulates the expression of one or more endogenous mogroside synthesis pathway genes. In contrast, control, non-transgenic, or unmodified plants or cells or tissues refer to plants or cells or tissues that are without such modifications (e.g., without the modifications to modulate the expression of one or more endogenous mogroside synthesis pathway genes). It is recognized that other exogenous or endogenous nucleic acid sequences or DNA fragments may also be incorporated into the plant cell. Agrobacterium-and biolistic-mediated transformation remain the two predominantly employed approaches. However, transformation may be performed by infection, transfection, microinjection, electroporation, microprojection, biolistics or particle bombardment, electroporation, silica/carbon fibers, ultrasound mediated, PEG mediated, calcium phosphate co-precipitation, polycation DMSO technique, DEAF dextran procedure, Agro and viral mediated (Caulimoriviruses, Geminiviruses, RNA plant viruses), liposome mediated and the like.

Transformation protocols as well as protocols for introducing polypeptides or polynucleotide sequences into plants may vary depending on the type of plant or plant cell, i.e., monocot or dicot, targeted for transformation. Methods for transformation are known in the art and include those set forth in U.S. Pat. Nos. 8,575,425; 7,692,068; 8,802,934; 7,541,517; each of which is herein incorporated by reference. See, also, Rakoczy-Trojanowska, M. (2002) Cell Mol Biol Lett. 7:849-858; Jones et al. (2005) Plant Methods 1:5; Rivera et al. (2012) Physics of Life Reviews 9:308-345; Bartlett et al. (2008) Plant Methods 4:1-12; Bates, G. W. (1999) Methods in Molecular Biology 111:359-366; Binns and Thomashow (1988) Annual Reviews in Microbiology 42:575-606; Christou, P. (1992) The Plant Journal 2:275-281; Christou, P. (1995) Euphytica 85:13-27; Tzfira et al. (2004) TRENDS in Genetics 20:375-383; Yao et al. (2006) Journal of Experimental Botany 57:3737-3746; Zupan and Zambryski (1995) Plant Physiology 107:1041-1047; Jones et al. (2005) Plant Methods 1:5.

Transformation may result in stable or transient incorporation of the nucleic acid into the cell. “Stable transformation” is intended to mean that the nucleotide construct introduced into a host cell integrates into the genome of the host cell and is capable of being inherited by the progeny thereof. “Transient transformation” is intended to mean that a polynucleotide is introduced into the host cell and does not integrate into the genome of the host cell.

Methods for transformation of chloroplasts are known in the art. See, for example, Svab et al. (1990) Proc. Natl. Acad. Sci. USA 87:8526-8530; Svab and Maliga (1993) Proc. Natl. Acad. Sci. USA 90:913-917; Svab and Maliga (1993) EMBO J. 12:601-606. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al. (1994) Proc. Natl. Acad. Sci. USA 91:7301-7305.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a nucleotide construct of the invention, for example, an expression cassette of the invention, stably incorporated into their genome.

In specific embodiments, the sequences provide herein can be targeted to specific site within the genome of the host cell or plant cell. Methods for targeting sequence to specific sites in the genome can include the use of engineered nucleases.

It is known in the art that it is possible to use a site-specific nuclease to make a DNA break in the genome of a living cell, and that such a DNA break can result in permanent modification of the genome via homologous recombination with a transgenic DNA sequence. The use of nucleases to induce a double-strand break in a target locus is known to stimulate homologous recombination, particularly of transgenic DNA sequences flanked by sequences that are homologous to the genomic target. In this manner, exogenous nucleic acids can be inserted into a target locus. For example, exogenous nucleic acids can be inserted into the genome of a Cucurbitaceae family plant in order to modulate the expression of at least one mogroside synthesis pathway gene.

It is known in the art that it is possible to use a site-specific nuclease to make a DNA break in the genome of a living cell, and that such a DNA break can result in permanent modification of the genome via mutagenic NHEJ repair or via homologous recombination with a transgenic DNA sequence. NHEJ can produce mutagenesis at the cleavage site, resulting in inactivation of the allele. NHEJ-associated mutagenesis may inactivate an allele via generation of early stop codons, frameshift mutations producing aberrant non-functional proteins, or could trigger mechanisms such as nonsense-mediated mRNA decay. The use of nucleases to induce mutagenesis via NHEJ can be used to target a specific mutation or a sequence present in a wild-type allele. Further, the use of nucleases to induce a double-strand break in a target locus is known to stimulate homologous recombination, particularly of transgenic DNA sequences flanked by sequences that are homologous to the genomic target. In this manner, exogenous nucleic acid sequences can be inserted into a target locus. Such exogenous nucleic acids can encode any sequence or polypeptide of interest, such as sequences that can modulate the expression of an endogenous mogroside synthesis pathway gene in order to increase the level of mogrosides in the plant.

Thus, in different embodiments, a variety of different types of nucleases are useful for practicing the invention. In one embodiment, the invention can be practiced using engineered recombinant meganucleases. In another embodiment, the invention can be practiced using a CRISPR system nuclease (e.g., CRISPR/Cas9 or RNA-guided nucleases such as Cpfl, MAD7, etc.), or CRISPR system nickase. Methods for making CRISPR and CRISPR Nickase systems that recognize and bind pre-determined DNA sites are known in the art, for example Ran, et al. (2013) Nat Protoc. 8:2281-308. In another embodiment, the invention can be practiced using TALENs or Compact TALENs. Methods for making TALE domains that bind to pre-determined DNA sites are known in the art, for example Reyon et al. (2012) Nat Biotechnol. 30:460-5. In another embodiment, the invention can be practiced using zinc finger nucleases (ZFNs). In a further embodiment, the invention can be practiced using megaTALs.

In some embodiments, the nucleases used to practice the invention are meganucleases. In particular embodiments, the nucleases used to practice the invention are single-chain meganucleases. A single-chain meganuclease comprises an N-terminal subunit and a C-terminal subunit joined by a linker peptide. Each of the two domains recognizes and binds to half of the recognition sequence (i.e., a recognition half-site) and the site of DNA cleavage is at the middle of the recognition sequence near the interface of the two subunits. DNA strand breaks are offset by four base pairs such that DNA cleavage by a meganuclease generates a pair of four base pair, 3′ single-strand overhangs.

In some embodiments, systems used to edit the genomes of plants include but are not limited to, engineered meganucleases (e.g., homing endonucleases) designed against the plant genomic sequence of interest (D'Halluin et al. 2013 Plant Biotechnol J); CRISPR-Cas9, alternative CRISPR editing systems known in the art (e.g., RNA-guided nucleases, such as Cpfl, MAD7, etc.), TALENs, and other technologies can be used for precise editing of genomes (e.g., Feng, et al. Cell Research 23:1229-1232, 2013, Podevin, et al. Trends Biotechnology, online publication, 2013, Wei et al., .1 Gen Genomics, 2013, Zhang et al (2013) WO 2013/026740); Cre-lox site-specific recombination (Dale et al. (1995) Plant J7:649-659; Lyznik, et al. (2007) Transgenic Plant J 1:1-9; FLP-FRT recombination (Li et al. (2009) Plant Physiol 151:1087-1095); Bxbl-mediated integration (Yau et al. Plant J (2011) 701:147-166); zinc-finger nuclease mediated integration (Wright el al. (2005) Plant J44:693-705); Cai et al. (2009) Plant Mol Biol 69:699-709); and homologous recombination (Lieberman-Lazarovich and Levy (2011) Methods Mol Biol 701: 51-65); Puchta (2002) Plant Mol Biol 48:173-182).

### 2.6 Breeding of Plants

Also disclosed herein are methods for breeding a plant, such as a plant (e.g., a Cucurbitaceae family plant, such as a watermelon (e.g., a Charleston Gray variety watermelon) plant or a cucumber plant) of the present disclosure having a genetic modification to modulate the expression of one or more endogenous mogroside synthesis pathway genes. A plant having a genetic modification to modulate the expression of one or more endogenous mogroside synthesis pathway genes may be regenerated from a plant cell or plant part of the present disclosure, wherein the genome of the plant cell or plant part is genetically-modified to modulate the expression of one or more endogenous mogroside synthesis pathway genes by the methods disclosed herein. Using conventional breeding techniques or self-pollination, one or more seeds may be produced from the plant that has a genetic modification to modulate the expression of one or more endogenous mogroside synthesis pathway genes. Such a seed, and the resulting progeny plant grown from such a seed, may contain modulated (e.g., increased or decreased) expression of one or more endogenous genes of the mogroside synthesis pathway, and therefore may be transgenic.

Plants having a genetic modification to modulate the expression of one or more endogenous mogroside synthesis pathway genes can be self-pollinated to provide one or more seeds for homozygous plants of the disclosure (homozygous for the modification in genome that results in modulated expression of one or more endogenous genes of the mogroside synthesis pathway) or crossed with non-modified plants or differently modified plants to provide seeds for heterozygous plants of the disclosure (heterozygous for the modification in the genome that results in modulated expression of one or more endogenous genes of the mogroside synthesis pathway). Both such homozygous and heterozygous plants are referred to herein as “progeny plants.” Progeny plants are plants having a genetic modification to modulate expression of one or more endogenous mogroside synthesis pathway genes, which descended from the original plant having modification in the genome that results in modulated (e.g., increased or decreased) expression of one or more endogenous genes of the mogroside synthesis pathway. Seeds produced using such a plant of the invention can be harvested and used to grow generations of plants having genetic modification to modulate expression of one or more endogenous mogroside synthesis pathway genes, e.g., progeny plants, of the invention, comprising the endogenous gene(s) of the mogroside synthesis pathway with modulated expression and optionally expressing a gene of agronomic interest (e.g., herbicide resistance gene). Descriptions of breeding methods that are commonly used for different crops can be found in one of several reference books, see, e.g., Allard, Principles of Plant Breeding, John Wiley & Sons, NY, U. of CA, Davis, Calif., 50-98 (1960); Simmonds, Principles of Crop Improvement, Longman, Inc., NY, 369-399 (1979); Sneep and Hendriksen, Plant breeding Perspectives, Wageningen (ed), Center for Agricultural Publishing and Documentation (1979); Fehr, Soybeans: Improvement, Production and Uses, 2nd Edition, Monograph, 16:249 (1987); Fehr, Principles of Variety Development, Theory and Technique, (Vol. 1) and Crop Species Soybean (Vol. 2), Iowa State Univ., Macmillan Pub. Co., NY, 360-376 (1987).

It will be readily apparent to those skilled in the art that other suitable modifications and adaptations of the methods of the invention described herein are obvious and may be made using suitable equivalents without departing from the scope of the invention or the embodiments disclosed herein. Having now described the invention in detail, the same will be more clearly understood by reference to the following examples, which are included for purposes of illustration only and are not intended to be limiting.

### EXAMPLES

The Examples below are merely illustrative, and are not intended to limit the scope of the disclosure provided herein in any way.

### Example 1. Activation of Mogroside Gene Homologs in Watermelon by Viral Promoter

The following example describes viral promoter-mediated activation of homologs of mogroside synthesis pathway genes in watermelon.

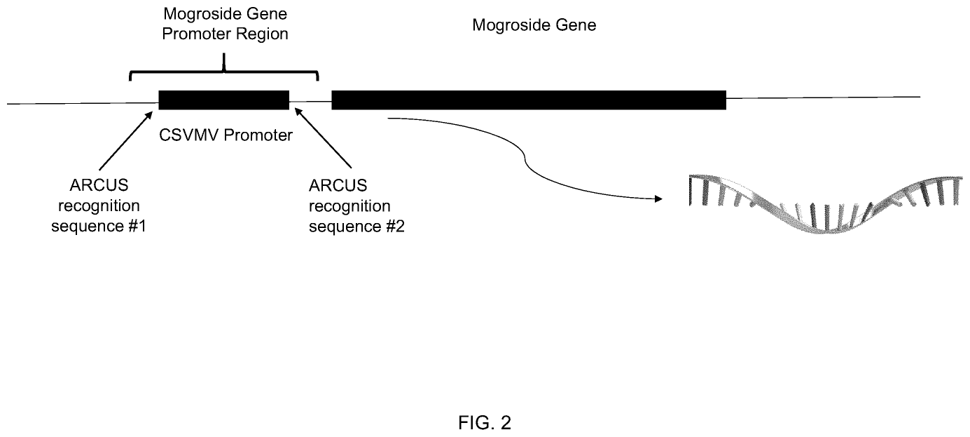

For activation of homologs of mogroside synthesis pathway genes in watermelon, an expression construct was used that contained a cassava vein mosaic virus (CSVMV) promoter operably linked to the mogroside gene homolog. A schematic of the expression construct is provided in FIG. 2.

First, a group of genes in watermelon that are evolutionarily conserved with mogroside bionsynthesis genes in monk fruit were identified. UGT72 (C1CG04O007540; SEQ ID NO: 7) and CYP87D18 (C1CG01G014540; SEQ ID NO: 3) were two of these genes. Both of these genes have very low expression levels in watermelon leaf and fruit tissues.

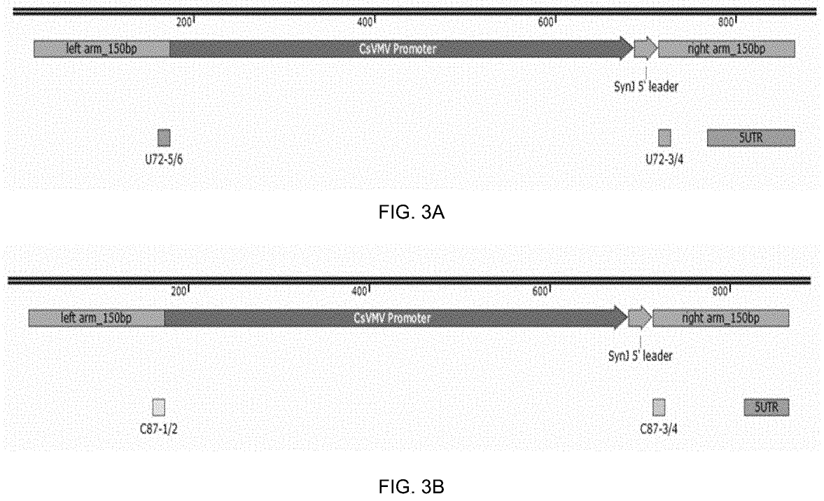

In order to demonstrate that these genes can be activated using genome editing, pairs of engineered meganucleases, referred to as ARCUS nucleases (Pair 1: U72 3-4 and U72 5-6; Pair 2: C87 1-2 and C87 3-4) were made to target the promoter region of the UGT72 and CYP87D18 genes, respectively. For each of the promoters, a specific repair template was designed to insert a CsVMV promoter, together with a SynJ 5′ leader sequence, between the pair of ARCUS nuclease recognition sites. These repair templates, delivered as double stranded DNA, consisted of the 540 bp CsVMV and SynJ 5′ sequences, as well as 150 bp of homology arms on both sides. The homology arms had matching sequences with the watermelon genomic region flanking the ARCUS nuclease recognition sites, as shown in FIGS. 3A-3B.

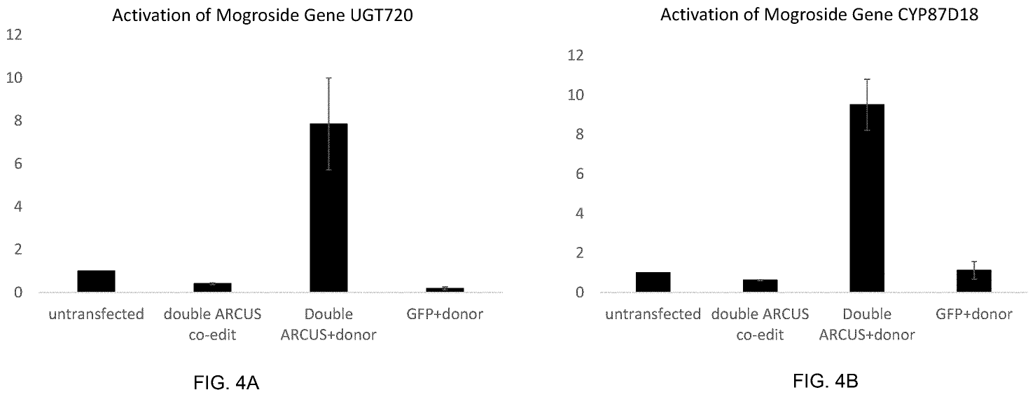

In a first case, mRNA encoding the U72 3-4 and U72 5-6 ARCUS nucleases were synthesized and mixed with the DNA of repair template that carry homology arms to the UGT72 gene (FIG. 3A). The mixture was then delivered to watermelon protoplast isolated from seedlings and incubated for 72 hours. As a control, a separate delivery was made with just the mRNA encoding the U72 3-4 and U72 5-6 ARCUS nucleases, without the DNA repair template. In a separate control, GFP mRNA was co-delivered with the DNA repair template. Each gene delivery was duplicated to ensure consistency. After 72 hours, the non-transfected protoplasts, the protoplasts delivered with just the ARCUS nucleases, and the protoplasts delivered with both the ARCUS nucleases and the repair templates were collected for total RNA extraction. Q-RT-PCR primers were designed to specifically amplify a region of UGT72 in the coding region. The expression of UGT72, from all samples, were investigated using Q-RT-PCR. The results are shown in FIG. 4A.

As described in FIG. 4A, compared to untransfected samples, insertion of CsVMV promoter and the SynJ 5′ leading sequence significantly increased gene expression. The increased gene expression was not observed in two control deliveries, where the DNA repair temples or the ARCUS enzymes were not combined.

In a second case, mRNA encoding the C87 1-2 and C87 3-4 ARCUS nucleases were synthesized and mixed with the DNA of repair template that carry homology arms to the CYP87 gene (FIG. 3B). The mixture was then delivered to watermelon protoplast isolated from seedlings and incubated for 72 hours. As a control, a separate delivery was made with just the mRNA encoding the C87 1-2 and C87 3-4 ARCUS nucleases, without the DNA repair template. In a separate control, GFP mRNA was co-delivered with the DNA repair template. Each gene delivery was duplicated to ensure consistency. After 72 hours, the non-transfected protoplasts, the protoplasts delivered with just the ARCUS nucleases, and the protoplasts delivered with both the ARCUS nucleases and the repair templates were collected for total RNA extraction. Q-RT-PCR primers were designed to specifically amplify a region of CYP87 in the coding region. The expression of CYP87, from all samples, were investigated using Q-RT-PCR. The results are shown in FIG. 4B.

As described in FIG. 4B, compared to untransfected samples, insertion of CsVMV promoter and the SynJ 5′ leading sequence, significantly increased gene expression. The increased gene expression was not observed in two control deliveries, where the DNA repair temples or the ARCUS enzymes were not combined.

Thus, the viral promoter could effectively activate homologs of mogroside synthesis pathway genes in watermelon.

A summary of all sequences disclosed herein is provided in the sequence summary table (Table 1) below.

TABLE 1
Sequence summary table

| Sequence identifier | Description |
|---|---|
| SEQ ID NO: 1 | C1CG00G001250 |
| | (homolog of mogroside synthesis pathway gene) |
| SEQ ID NO: 2 | C1CG00G003500 |
| | (homolog of mogroside synthesis pathway gene) |
| SEQ ID NO: 3 | C1CGO1G0 14540 |
| | (homolog of mogroside synthesis pathway gene) |
| SEQ ID NO: 4 | C1CG01G014560 |
| | (homolog of mogroside synthesis pathway gene) |
| SEQ ID NO: 5 | C1CG02G024130 |
| | (homolog of mogroside synthesis pathway gene) |
| SEQ ID NO: 6 | C1CG02G024140 |
| | (homolog of mogroside synthesis pathway gene) |
| SEQ ID NO: 7 | C1CG04G007540 |
| | (homolog of mogroside synthesis pathway gene) |
| SEQ ID NO: 8 | C1CGO5G0 17620 |
| | (homolog of mogroside synthesis pathway gene) |
| SEQ ID NO: 9 | C1CG06G001600 |
| | (homolog of mogroside synthesis pathway gene) |
| SEQ ID NO: 10 | C1CG08G016750 |
| | (homolog of mogroside synthesis pathway gene) |
| SEQ ID NO: 11 | C1CG09G000010 |
| | (homolog of mogroside synthesis pathway gene) |
| SEQ ID NO: 12 | C1CG09G001740 |
| | (homolog of mogroside synthesis pathway gene) |
| SEQ ID NO: 13 | C1CG10G011000 |
| | (homolog of mogroside synthesis pathway gene) |
| SEQ ID NO: 14 | C1CG10G011060 |
| | (homolog of mogroside synthesis pathway gene) |
| SEQ ID NO: 15 | CDS |
| SEQ ID NO: 16 | CYP87D18 |
| SEQ ID NO: 17 | EPH3 |
| SEQ ID NO: 18 | SQE |
| SEQ ID NO: 19 | UGT720 |
| SEQ ID NO: 20 | UGT94 |
| SEQ ID NO: 21 | CYP102801 |
| SEQ ID NO: 22 | I-Crel |

The contents of all references, patents, pending patent applications, and publications cited throughout this application are hereby expressly incorporated by reference herein in their entirety.

Those skilled in the art will recognize, or be able to ascertain using no more than routine experimentation, many equivalents to the specific embodiments of the invention described herein. Such equivalents are intended to be encompassed by the following claims.

1. A method for increasing the production of one or more mogrosides by a cell, the method comprising genetically-modifying the genome of the cell to modulate the expression of one or more endogenous mogroside synthesis pathway genes.

2. The method of claim 1, wherein the one or more endogenous mogroside synthesis pathway genes are selected from an endogenous gene encoding cucurbitadienol synthase (CDS), an endogenous gene encoding cytochrome P450 enzyme 87D18 (CYP87D18), an endogenous gene encoding epoxide hydrolase 3 (EPH3), an endogenous gene encoding squalene epoxidase 1 (SQE1), an endogenous gene encoding UDP glycosyltransferase 720 (UGT720), an endogenous gene encoding UDP glycosyltransferase 94 (UGT94), or one or more homologs thereof.

3. The method of claim 1, wherein the one or more endogenous mogroside synthesis pathway genes are selected from endogenous genes encoding polypeptide sequences having at least 80% sequence identity to the amino acid sequence set forth in any one of SEQ ID NOs: 1-14 and 21.

4. The method of claim 1, wherein the cell is from a Cucurbitaceae family plant.

5.-6. (canceled)

7. The method of claim 1, wherein modulating the expression of the one or more endogenous mogroside synthesis pathway genes comprises:
a) modification of promoter sequences of the genes;
b) modulation of expression of one or more transcription factor genes;
c) modifying negative regulatory sequences of the genes;
d) insertion of one or more functional promoters into the genome of the cell, wherein following said insertion, the one or more functional promoters are operably linked to the one or more endogenous mogroside synthesis pathway genes; or
e) increasing the expression of said one or more endogenous mogroside synthesis pathway genes.

8.-18. (canceled)

19. The method of claim 1, wherein the one or more mogrosides is selected from mogrol, mogroside I-A1, mogroside I-E1, mogroside IIA, mogroside IIB, mogroside IIE, 7-oxomogroside IIE, 11-oxomogroside A1, mogroside III, mogroside III A1, mogroside III A2, mogroside Mx, 11-deoxymogroside III, mogroside IV, mogroside IV-A, 11-oxomogroside IV-A, siamenoside I, mogroside V, 7-oxomogroside V, 11-oxomogroside V, mogroside VI, and mogroside VII.

20. The method of claim 19, wherein the one or more mogroside is mogroside V.

21.-23. (canceled)

24. A method for producing a plant comprising increased levels of one or more mogrosides, the method comprising genetically-modifying the genome of a plant cell or plant part to modulate the expression of one or more endogenous mogroside synthesis pathway genes, and growing a plant from said plant cell or plant part, wherein said plant comprises increased levels of one or more mogrosides compared to a control plant.

25. The method of claim 24, wherein the one or more endogenous mogroside synthesis pathway genes are selected from an endogenous gene encoding cucurbitadienol synthase (CDS), an endogenous gene encoding cytochrome P450 enzyme 87D18 (CYP87D18), an endogenous gene encoding epoxide hydrolase 3 (EPH3), an endogenous gene encoding squalene epoxidase 1 (SQE1), an endogenous gene encoding UDP glycosyltransferase 720 (UGT720), an endogenous gene encoding UDP glycosyltransferase 94 (UGT94), or one or more homologs thereof.

26. The method of claim 24 or 25, wherein the one or more endogenous mogroside synthesis pathway genes are selected from endogenous genes encoding polypeptide sequences having at least 80% sequence identity to the amino acid sequence set forth in any one of SEQ ID NOs: 1-14 and 21.

27. The method of claim 24, wherein genetically-modifying the genome of a plant cell or plant part to modulate the expression of the one or more endogenous mogroside synthesis pathway genes comprises;
a) modification of promoter sequences of the genes;
b) modulation of expression of one or more transcription factor genes;
c) modifying negative regulatory sequences of the genes;
d) insertion of one or more functional promoters into the genome of the plant cell or plant part, wherein following said insertion, the one or more functional promoters are operably linked to said one or more mogroside synthesis pathway genes; or
e) increasing the expression of the one or more endogenous mogroside synthesis pathway genes.

28.-38. (canceled)

39. The method of claim 24, wherein the one or more mogrosides is selected from mogrol, mogroside I-A1, mogroside I-E1, mogroside IIA, mogroside IIB, mogroside IIE, 7-oxomogroside IIE, 11-oxomogroside A1, mogroside III, mogroside III A1, mogroside III A2, mogroside Mx, 11-deoxymogroside III, mogroside IV, mogroside IV-A, 11-oxomogroside IV-A, siamenoside I, mogroside V, 7-oxomogroside V, 11-oxomogroside V, mogroside VI, and mogroside VII.

40. The method of claim 39, wherein the one or more mogrosides is mogroside V.

41. (canceled)

42. The method of claim 24, wherein the plant is a Cucurbitaceae family plant.

43. The method of claim 42, wherein the plant is a watermelon plant or cucumber plant.

44. The method of claim 43, wherein the plant is a Charleston Gray variety watermelon plant.

45. A plant or plant part comprising increased levels of at least one mogroside compared to a control plant, wherein the genome of the plant comprises a genetic modification to modulate the expression of one or more endogenous mogroside synthesis pathway genes.

46. The plant or plant part of claim 45, wherein the one or more endogenous mogroside synthesis pathway genes are selected from an endogenous gene encoding cucurbitadienol synthase (CDS), an endogenous gene encoding cytochrome P450 enzyme 87D18 (CYP87D18), an endogenous gene encoding epoxide hydrolase 3 (EPH3), an endogenous gene encoding squalene epoxidase 1 (SQE1), an endogenous gene encoding UDP glycosyltransferase 720 (UGT720), an endogenous gene encoding UDP glycosyltransferase 94 (UGT94), or one or more homologs thereof.

47. The plant or plant part of claim 45 or 46, wherein the one or more mogroside synthesis pathway genes are selected from endogenous genes encoding polypeptide sequences having at least 80% sequence identity to the amino acid sequence set forth in any one of SEQ ID NOs: 1-14 and 21.

48. The plant or plant part of claim 45, wherein said genetic modification to modulate the of expression of one or more endogenous mogroside synthesis pathway genes comprises:
a) a genetic modification of a promoter sequence of the one or more endogenous mogroside synthesis pathway genes;
b) modulation of expression of one or more transcription factor genes;
c) modifying negative regulatory sequences of the genes;
d) insertion of one or more functional promoters into the genome of the plant cell or plant part, wherein following said insertion, the one or more functional promoters are operably linked to said one or more mogroside synthesis pathway genes; or
e) increasing the expression of the one or more endogenous mogroside synthesis pathway genes.

49.-59. (canceled)

60. The plant or plant part of claim 45, wherein the one or more mogrosides is selected from mogrol, mogroside I-A1, mogroside I-E1, mogroside IIA, mogroside IIB, mogroside IIE, 7-oxomogroside IIE, 11-oxomogroside A1, mogroside III, mogroside III A1, mogroside III A2, mogroside Mx, 11-deoxymogroside III, mogroside IV, mogroside IV-A, 11-oxomogroside IV-A, siamenoside I, mogroside V, 7-oxomogroside V, 11-oxomogroside V, mogroside VI, and mogroside VII.

61. The plant or plant part of claim 60, wherein the one or more mogrosides is mogroside V.

62. (canceled)

63. The plant or plant part of claim 45, wherein the plant is a Cucurbitaceae family plant.

64. The plant or plant part of claim 63, wherein the plant is a watermelon plant or cucumber plant.

65. The plant or plant part of claim 64, wherein the plant is a Charleston Gray variety watermelon plant.

66. An extract, seed, plant concentrate, plant powder or plant biomass from the plant or plant part of claim 45.

67.-71. (canceled)

72. The plant part of claim 45, wherein the plant part has a sweetness index of 200-450×compared to cane sugar.

73. The plant part of claim 45, wherein the plant part has a glycemic index (GI) of 5 or less.

74. The plant part of claim 45, wherein the plant part is pulp or a juice.

75. (canceled)

76. A seed of the plant of claim 45, wherein the seed comprises the genetic modification to modulate the expression of the one or more endogenous mogroside synthesis pathway genes.

77.-97. (canceled)

98. A progeny plant of the plant of claim 45, wherein the expression of one or more endogenous mogroside synthesis pathway genes is modulated in said progeny plant.

99. A seed from the progeny plant of claim 98, wherein the expression of the one or more endogenous mogroside synthesis pathway genes is modulated in said seed from said progeny plant.

100. A method for producing a progeny plant having a genetic modification to modulate expression of one or more endogenous mogroside synthesis pathway genes, said method comprising crossing the plant of claim 45 with a plant that has not been genetically-modified to modulate the expression of an endogenous mogroside synthesis pathway genes in order to produce a progeny plant having modulated expression of one or more endogenous mogroside synthesis pathway genes compared to a control plant.

101.-102. (canceled)
